(19) United States
(12) Patent Application Publication
(10) Pub. No.: US 2023/0226049 A1
(43) Pub. Date: Jul. 20, 2023

(54) ACALABRUTINIB MALEATE DOSAGE FORMS

(71) Applicant: Acerta Pharma B.V., Oss (NL)

(72) Inventors: Paul BETHEL, Macclesfield (GB); John BLYTH, Macclesfield (GB); Steve COSGROVE, Macclesfield (GB); Michael GOLDEN, Macclesfield (GB); James MANN, Macclesfield (GB); Xavier Jacques Henri PEPIN, Macclesfield (GB); Andrew ROBBINS, Macclesfield (GB); David SIMPSON, Macclesfield (GB)

(21) Appl. No.: 18/001,740

(22) PCT Filed: Jun. 18, 2021
(86) PCT No.: PCT/EP2021/066629

§ 371 Date: Dec. 14, 2022

Related U.S. Application Data
(60) Provisional application No. 63/041,197, filed on Jun. 19, 2020.

(51) Int. Cl. A61K 31/4985; A61K 9/00; A61K 9/20

(52) U.S. Cl. CPC A61K 31/4985; A61K 9/0053; A61K 9/2013; A61K 9/2018; A61K 9/2054

(57) ABSTRACT

The present disclosure relates, in general, to: (a) solid pharmaceutical dosage forms comprising acalabrutinib maleate; (b) methods of using such pharmaceutical dosage forms to treat B-cell malignancies and/or other conditions; (c) kits comprising such pharmaceutical dosage forms and, optionally, a second pharmaceutical dosage form comprising another therapeutic agent; (d) methods for the preparation of such pharmaceutical dosage forms; and (e) pharmaceutical dosage forms prepared by such methods.

### FIELD OF THE INVENTION

The present disclosure relates, in general, to: (a) solid pharmaceutical dosage forms comprising acalabrutinib maleate; (b) methods of using such pharmaceutical dosage forms to treat B-cell malignancies and/or other conditions; (c) kits comprising such pharmaceutical dosage forms and, optionally, a second pharmaceutical dosage form comprising another therapeutic agent; (d) methods for the preparation of such pharmaceutical dosage forms; and (e) pharmaceutical dosage forms prepared by such methods.

### BACKGROUND OF THE INVENTION

Acalabrutinib is a selective, covalent Bruton Tyrosine Kinase (“BTK”) inhibitor. It is the active pharmaceutical ingredient in the drug product CALQUENCE® which has been approved in several countries (including the United States, Canada, and Australia) for the treatment of chronic lymphocytic leukemia, small lymphocytic leukemia, and mantle cell lymphoma. CALQUENCE® is marketed as a capsule dosage form containing 100 mg of crystalline acalabrutinib free base (specifically, the Form A anhydrate). International Publication WO2017/002095 reports the Form A anhydrate, additional crystalline acalabrutinib free base forms, and crystalline acalabrutinib salt forms including, for example, citrate, fumarate, gentisate, maleate, oxalate, phosphate, sulfate, and L-tartrate salts of acalabrutinib. The prescribing information for CALQUENCE® recommends avoiding co-administration with gastric acid reducing agents because such agents can decrease acalabrutinib plasma concentrations. Accordingly, there is a need for acalabrutinib pharmaceutical dosage forms that reduce the potential impact of gastric acid reducing agents on acalabrutinib plasma concentrations when co-administered with the acalabrutinib dosage form.

### BRIEF DESCRIPTION OF THE INVENTION

In one aspect, the disclosure relates to solid pharmaceutical dosage forms comprising from about 75 mg to about 125 mg (free base equivalent weight) of acalabrutinib maleate and at least one pharmaceutically acceptable excipient for oral administration to a human, wherein the dosage form satisfies the following conditions:
at least about 75% of the acalabrutinib maleate is dissolved within about 30 minutes as determined in an in vitro dissolution test conducted using a USP Dissolution Apparatus 2 (Paddle Apparatus), 900 mL dissolution volume, 0.1N hydrochloric acid dissolution medium, and paddle rotation of 50 RPM; and
at least about 75% of the acalabrutinib maleate is dissolved within about 60 minutes as determined in an in vitro dissolution test conducted using a USP Dissolution Apparatus 2 (Paddle Apparatus), 900 mL dissolution volume, 5 mM phosphate pH 6.8 dissolution medium, and paddle rotation of 75 RPM.

In further aspects, the solid pharmaceutical dosage forms comprise from about 75 mg to about 100 mg (free base equivalent weight) of acalabrutinib maleate. In still further aspects, the acalabrutinib maleate is present as acalabrutinib maleate monohydrate, such as crystalline acalabrutinib maleate monohydrate Form A.

In another aspect, the present disclosure relates to the above-described solid pharmaceutical dosage forms wherein the dissolution rate of the acalabrutinib maleate in the 5 mM phosphate pH 6.8 dissolution medium does not decrease by more than 20% from its initial dissolution rate after storage of the dosage form in appropriate packaging for six months at 40° C. and 75% relative humidity.

In another aspect, the present disclosure relates to one or more of the above-described solid pharmaceutical dosage forms wherein no more than about 5% (w/w) of the acalabrutinib maleate present in the dosage form degrades after storage of the dosage form in appropriate packaging for six months at 40° C. and 75% relative humidity.

In another aspect, the present disclosure relates to one or more of the above-described solid pharmaceutical dosage forms wherein the dosage form is bioequivalent to a 100 mg Calquence® capsule when orally administered to a fasting human subject who has not been administered a gastric acid reducing agent, wherein the dosage form is bioequivalent when the confidence interval of the relative mean Cₘₐₓ, AUC₍₀₋ₜ₎, and AUC_{(0-∞)} of the dosage form relative to the 100 mg Calquence® capsule is within 80% to 125%.

In another aspect, the present disclosure relates to one or more of the above-described solid pharmaceutical dosage forms wherein the dosage form, when administered twice daily to a population of fasting human subjects, satisfies one or more of the following pharmacokinetic conditions for acalabrutinib:
the average Cₘₐₓ value in the population of human subjects is from about 400 ng/mL to about 900 ng/mL;
the average AUC₍₀₋₂₄₎ value in the population of human subjects is from about 350 ng•hr/mL to about 1900 ng•hr/mL; and/or
the average AUC_{(0-∞)} value in the population of human subjects is from about 350 ng•hr/mL to about 1900 ng•hr/mL.

In another aspect, the present disclosure relates to one or more of the above-described solid pharmaceutical dosage forms wherein the dosage form, when administered twice daily to a human subject, provides a median steady state Bruton tyrosine kinase occupancy of at least about 90% in peripheral blood mononuclear cells.

In another aspect, the present disclosure relates to one or more of the above-described solid pharmaceutical dosage forms wherein the dosage form comprises:
acalabrutinib maleate in an amount from about 15% to about 55% by weight of the dosage form;
at least one diluent in an amount from about 10% to about 70% by weight of the dosage form;
at least one disintegrant in an amount from about 0.5% to about 15% by weight of the dosage form; and
at least one lubricant in an amount from about 0.25% to about 4% by weight of the dosage form; and
wherein the sum of the individual amounts equals 100% of the total weight of the dosage form.

In another aspect, the present disclosure relates to one or more of the above-described solid pharmaceutical dosage forms wherein the dosage form comprises:
acalabrutinib maleate monohydrate in an amount from about 30% to about 35% by weight (free base equivalent weight) of the dosage form;
mannitol in an amount from about 30% to about 35% by weight of the dosage form; microcrystalline cellulose in an amount from about 25% to about 30% by weight of the dosage form;
hydroxypropyl cellulose in an amount from about 3% to about 7% by weight of the dosage form; and
sodium stearyl fumarate in an amount from about 1% to about 4% by weight of the dosage form; and
wherein the sum of the individual amounts equals 100% of the total weight of the dosage form.

### BRIEF DESCRIPTION OF THE DRAWINGS

FIG. 1 is a representative XRPD diffractogram for crystalline acalabrutinib maleate monohydrate Form A.

FIG. 2 shows dissolution profiles for phosphate, oxalate, and maleate salts of acalabrutinib in simulated gastric fluid/FaSSIF-V2 media.

FIG. 3 shows dissolution profiles for phosphate, oxalate, and maleate salts of acalabrutinib in deionized water/FaSSIF-V2 media.

FIG. 4 is a dynamic vapour sorption plot for an acalabrutinib phosphate salt.

FIG. 5 is a thermogravimetric analysis plot for an acalabrutinib phosphate salt.

FIG. 6 is an XRPD diffractogram for an acalabrutinib phosphate salt.

FIG. 7 is a thermogravimetric analysis plot for an acalabrutinib oxalate salt.

FIG. 8 is a dynamic vapour sorption plot for an acalabrutinib oxalate salt.

FIG. 9A is a thermogravimetric analysis plot for an acalabrutinib maleate salt.

FIG. 9B is a thermogravimetric analysis plot for an acalabrutinib maleate salt carried out under an alternative set of conditions.

FIG. 10A is a dynamic vapour sorption plot for a first sample of acalabrutinib maleate salt.

FIG. 10B is a dynamic vapour sorption plot for a second, higher quality sample of acalabrutinib maleate salt.

FIG. 11 shows dissolution profiles for micronized and unmilled acalabrutinib maleate salts in simulated gastric fluid/FaSSIF-V2 media.

FIG. 12 shows dissolution profiles for micronized and unmilled acalabrutinib maleate salts in deionized water/FaSSIF-V2 media.

FIG. 13 shows the solubility versus final pH values for acalabrutinib maleate and acalabrutinib free base in a variety of buffered solutions.

FIG. 14 shows dissolution profiles obtained from a low pH test under sink conditions for acalabrutinib maleate tablets T16, T17, and T18, and acalabrutinib free base capsule C1.

FIG. 15 shows dissolution profiles obtained from a neutral pH low ionic strength test under sink conditions for acalabrutinib maleate tablets T16, T17, and T18.

FIG. 16 shows dissolution profiles obtained from a neutral pH high ionic strength test for acalabrutinib maleate tablet T13 and acalabrutinib free base capsule C2.

FIG. 17 shows dissolution profiles obtained from a neutral medium with no buffer capacity (i.e., conditions similar to a proton pump inhibitor-treated stomach) for acalabrutinib maleate tablet T1 and acalabrutinib free base capsule C1.

FIG. 18 shows dissolution profiles obtained from a neutral medium with no buffer capacity for acalabrutinib maleate tablet T13 and acalabrutinib free base capsule C1.

FIG. 19 shows a dissolution profile under pH shift conditions for acalabrutinib maleate tablet T19.

FIG. 20 shows dissolution profiles under pH shift conditions for acalabrutinib maleate tablet T19 and acalabrutinib free base capsule C3.

FIG. 21 is a plot of acalabrutinib cumulative fraction available (%) versus time (minutes) for acalabrutinib maleate tablet T19 and acalabrutinib free base capsule C2 when evaluated in a TIM-1 system under gastric conditions associated with an acidic gastric compartment and also under gastric conditions associated with dosing in combination with a proton pump inhibitor or acid reducing agent.

FIG. 22 shows the particle size distributions for acalabrutinib maleate tablets T10 (D(ᵥ, _{0.9)} ≈ 150 µm), T11 (D_{(v, 0.9)} ≈ 16 µm), T13 (D_{(v, 0.9)} ≈ 500 µm), and T15 (D_{(v, 0.9)} ≈ 70 µm) .

FIG. 23 shows the dissolution profiles in a 5 mM sodium phosphate buffer medium for acalabrutinib maleate tablets T10, T11, T13, and T15 (drug loading of 26 weight%).

FIG. 24 shows the dissolution profiles in a 5 mM sodium phosphate buffer medium for acalabrutinib maleate tablets T9, T2, and T14 (drug loading of 43 weight%).

FIG. 25 reports the results of an in vivo study in a dog model to measure AUC₍₀₋₂₄₎ values for acalabrutinib free base and acalabrutinib maleate when co-administered with omeprazole.

FIG. 26 shows the dissolution profiles in a deionized water medium for several binary mixes of disintegrants and acalabrutinib maleate (1:5 ratio).

FIG. 27 shows the dissolution profiles in a deionized water medium for several binary mixes of lubricants and acalabrutinib maleate (1:15).

FIG. 28 shows the dissolution profiles in a deionized water medium for tablet cores T2 and T3.

FIG. 29 shows the dissolution profiles in a deionized water medium for tablet cores T6 and T8.

FIG. 30 shows the dissolution profiles in a deionized water medium for tablet cores T4 and T5.

FIG. 31 provides a schematic overview of a process for preparing the acalabrutinib maleate tablet T21 of Example 4.

### DETAILED DESCRIPTION OF THE INVENTION

### I. Definitions

Unless defined otherwise, all technical and scientific terms used herein have the same meaning as is commonly understood by one of skill in the art to which this invention belongs.

When ranges are used to describe, for example, amounts, all combinations and subcombinations of ranges and specific embodiments are intended to be included.

The singular forms “a,” “an” and “the” include plural referents unless the context clearly dictates otherwise.

Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. In many instances, the term “about” may include numbers that are rounded to the nearest significant figure.

The term “acalabrutinib” refers to the International Nonproprietary Name (INN) for the compound 4-{8-amino-3-[(2S)-1-(but-2-ynoyl)pyrrolidin-2-yl]imidazo[1,5-a]pyrazin-1-yl}-N-(pyridin-2-yl)benzamide which has the chemical structure shown below:

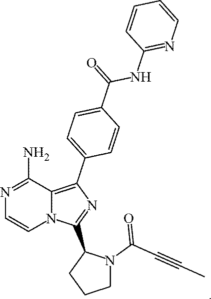

International Publication WO2013/010868 discloses acalabrutinib (Example 6) and describes the synthesis of acalabrutinib. International Publication WO2020/043787 further describes the synthesis of acalabrutinib. International Publication WO2013/010868 and International Publication WO2020/043787 are each incorporated by reference in their entirety.

The term “acalabrutinib maleate monohydrate” refers to crystalline acalabrutinib maleate monohydrate, including the crystalline Form A of acalabrutinib maleate monohydrate. Example 6.2 of International Publication No. WO2017/002095 describes the preparation of the crystalline Form A of acalabrutinib maleate monohydrate. International Publication No. WO2017/002095 is incorporated by reference in its entirety. Acalabrutinib maleate monohydrate Form A also can be referred to by the alternative nomenclature of acalabrutinib maleate monohydrate Form 1. Unless otherwise stated, any reference in this disclosure to an amount of acalabrutinib, acalabrutinib maleate, or acalabrutinib maleate monohydrate is based on the acalabrutinib free base equivalent weight. For example, 100 mg refers to 100 mg of acalabrutinib free base or an equivalent amount of acalabrutinib maleate or acalabrutinib maleate monohydrate.

The term “ACP-5862” refers to the compound 4-[8-amino-3-[4-(but-2-ynoylamino)butanoyl]imidazo[1,5-a]pyrazin-1-yl]-N-pyridin-2-ylbenzamide which has the chemical structure shown below:

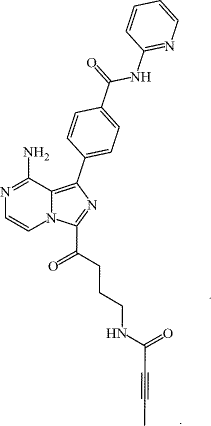

ACP-5862 is an active metabolite of acalabrutinib.

The term “AUC₍₀₋₂₄₎” refers to the area under the plasma concentration-time curve from the time 0 (time of dosing) to 24 hours after dosing, as calculated by the linear trapezoidal method.

The term “AUC_{(0-∞)}” refers to the area under the plasma concentration-time curve from the time 0 (time of dosing) to infinity (∞), as calculated by the linear trapezoidal method.

The term “BID” means bis in die, twice a day, or twice daily.

The term “Cₘₐₓ” refers to the maximum observed plasma concentration over the entire sampling period.

The terms “co-administration,” “in combination with,” and “combination” can refer to administration of two or more therapeutic agents. In one aspect, “combination” can refer to simultaneous administration (e.g., administration of both agents in separate dosage forms, but at substantially the same time). In a further aspect of the invention, “combination” can refer to sequential administration (e.g., where a first agent is administered, followed by a delay, followed by administration of a second or further agent). Where the administration is sequential, the delay in administering the later component should be neither too long nor too short, so as not to lose the benefit of the combination.

Unless the context requires otherwise, the terms “comprise,” “comprises,” and “comprising” are used on the basis and clear understanding that they are to be interpreted inclusively, rather than exclusively, and that applicant intends each of those words to be so interpreted in construing this patent, including the claims below.

The term “crystalline” as applied to acalabrutinib, acalabrutinib maleate, or acalabrutinib maleate monohydrate refers to a solid-state form wherein the molecules are arranged to form a distinguishable crystal lattice (i) comprising distinguishable unit cells, and (ii) yielding diffraction peaks when subjected to X-ray radiation.

The term “crystalline purity” means the crystalline purity of acalabrutinib, acalabrutinib maleate, or acalabrutinib maleate monohydrate with respect to a particular crystalline form as determined by X-ray powder diffraction analytical methods.

The term “crystallization” as used throughout this application can refer to crystallization and/or recrystallization depending upon the applicable circumstances relating to the preparation of acalabrutinib, acalabrutinib maleate, or acalabrutinib maleate monohydrate.

The terms “D_{(0.1)}” and “D_{(v,} _{0.1})” as used throughout this application mean that 10% of the total volume of material in the sample has a particle size diameter below the specified value as determined by laser diffraction.

The terms “D_{(0.5)}” and “D_{(v,} _{0.5)}” as used throughout this application mean that 50% of the total volume of material in the sample has a particle size diameter below the specified value as determined by laser diffraction.

The terms “D_{(0.9)}” and “D_{(v,} _{0.9)}” as used throughout this application mean that 90% of the total volume of material in the sample has a particle size diameter below the specified value as determined by laser diffraction.

The term “pharmaceutically acceptable” (such as in the recitation of a “pharmaceutically acceptable diluent” or a “pharmaceutically acceptable disintegrant”) refers to a material that is compatible with administration to a subject, e.g., the material does not cause an undesirable biological effect. Examples of pharmaceutically acceptable excipients are described in the “Handbook of Pharmaceutical Excipients,” Rowe et al., Ed. (Pharmaceutical Press, 7th Ed., 2012).

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. Except insofar as any conventional pharmaceutically acceptable carrier or pharmaceutically acceptable excipient is incompatible with acalabrutinib, acalabrutinib maleate, or acalabrutinib maleate monohydrate, its use in the therapeutic compositions of the invention is contemplated.

The term “Q” means the quantity (Q) of an active substance in a sample that is dissolved in a specified time, expressed as a percentage of the total amount of the active substance present in the sample.

The term “QD” means quaque die, once a day, or once daily.

The term “Tₘₐₓ” refers to the time of the maximum observed plasma concentration (Cmax).

The terms “treat,” “treating,” and “treatment” refer to ameliorating, suppressing, eradicating, reducing the severity of, decreasing the frequency of incidence of, reducing the risk of, or delaying the onset of the condition.

The abbreviations listed in Table 1 below have the meanings indicated in that table.

TABLE 1

| | |
|---|---|
| ABBREVIATION | MEANING |
| DC | Direct Compression |
| DLBCL | Diffuse Large B-Cell Lymphoma |
| DVS | Dynamic vapour sorption |
| FaSSIF | Fasted State Simulated Intestinal Fluid |
| g | gram |
| g/mol | Gram/mole |
| h | hour |
| HDPE | High-density polyethylene |
| HPLC | High-performance liquid chromatography |
| kg | kilogram |
| kN | Kilonewton |
| L | Liter |
| µl | Microliter |
| µm | Micron |
| µM | Micromolar |
| mg | Milligram(s) |
| mL | Milliliter |
| mm | Millimeter |
| nM | Nanomolar |
| NMT | Mot more than |
| PBPK | Physiological Based Pharmacokinetics |
| PK | Pharmacokinetic(s) |
| PPI | Proton Pump Inhibitor |
| P-PSD | Product particle size distribution |
| XRPD | Powder X-ray diffraction |
| RH | Relative humidity |
| RPM | Revolutions Per Minute |
| RRT | Relative retention time |
| TGA | Thermogravimetric analysis |
| USP | United States Pharmacopeia |
| w/w | Weight/weight |

### II. Solid Dosage Forms

The present disclosure relates, in part, to solid pharmaceutical dosage forms comprising acalabrutinib maleate, particularly crystalline acalabrutinib maleate monohydrate. According to the Biopharmaceutics Classification System (“BCS”), acalabrutinib is a BCS Class II drug substance which means that it exhibits good permeability but low solubility in the gastrointestinal tract. See Pepin, X. J. H., et al., “Bridging in vitro dissolution and in vivo exposure for acalabrutinib. Part II. A mechanistic PBPK model for IR formulation comparison, proton pump inhibitor drug interactions, and administration with acidic juices,” European Journal of Pharmaceutics and Biopharmaceutics 142: 435-448 (2019). The bioavailability of BCS Class II drug substances, including acalabrutinib, generally is limited by their dissolution rate and/or solvation. In addition, acalabrutinib free base exhibits pH-dependent solubility with solubility decreasing as pH increases up to the maximum basic pKa, (i.e., around pH 6 where acalabrutinib is largely un-ionized). Increasing the stomach pH of a subject taking CALQUENCE® (e.g., in a subject also taking a proton pump inhibitor or other gastric acid reducing agent) can reduce the solubility of acalabrutinib in the stomach and potentially result in lower bioavailability and/or greater intra- and inter-subject variability in acalabrutinib pharmacokinetics. The present disclosure relates to the unexpected finding that the solid pharmaceutical dosage forms containing acalabrutinib maleate as described below have acceptable physical and pharmacological properties (e.g., dissolution, stability, manufacturability, pharmacokinetics, etc.) and, while substantially bioequivalent to the currently marketed CALQUENCE® capsule dosage form in normal acidic stomach conditions, provide less variability in acalabrutinib pharmacokinetics over a broader range of stomach pH conditions. These solid dosage forms provide an additional therapeutic option for treating conditions including B-cell malignancies such as chronic lymphocytic leukemia, small lymphocytic leukemia, and mantle cell lymphoma.

In some embodiments, the present disclosure relates, in part, to solid pharmaceutical dosage forms comprising from about 75 mg to about 125 mg (free base equivalent weight) of acalabrutinib maleate and at least one pharmaceutically acceptable excipient for oral administration to a human, wherein the dosage form satisfies the following conditions:
at least about 75% of the acalabrutinib maleate is dissolved within about 30 minutes as determined in an in vitro dissolution test conducted using a USP Dissolution Apparatus 2 (Paddle Apparatus), 900 mL dissolution volume, 0.1 N hydrochloric acid dissolution medium, and paddle rotation of 50 RPM; and
at least about 75% of the acalabrutinib maleate is dissolved within about 60 minutes as determined in an in vitro dissolution test conducted using a USP Dissolution Apparatus 2 (Paddle Apparatus), 900 mL dissolution volume, 5 mM phosphate pH 6.8 dissolution medium, and paddle rotation of 75 RPM.

The 0.1 N hydrochloric acid dissolution medium is believed to be representative of the fasted stomach while the 5 mM phosphate pH 6.8 dissolution is believed to be representative of the worst-case scenario of a stomach treated with a gastric acid reducing agent. In one aspect, the dosage form satisfies the following conditions:
at least about 75% of the acalabrutinib maleate is dissolved within about 20 minutes as determined in an in vitro dissolution test conducted using a USP Dissolution Apparatus 2 (Paddle Apparatus), 900 mL dissolution volume, 0.1 N hydrochloric acid dissolution medium, and paddle rotation of 50 RPM; and
at least about 75% of the acalabrutinib maleate is dissolved within about 45 minutes as determined in an in vitro dissolution test conducted using a USP Dissolution Apparatus 2 (Paddle Apparatus), 900 mL dissolution volume, 5 mM phosphate pH 6.8 dissolution medium, and paddle rotation of 75 RPM.

In another aspect, the dosage form satisfies the following conditions:
at least about 80% of the acalabrutinib maleate is dissolved within about 20 minutes as determined in an in vitro dissolution test conducted using a USP Dissolution Apparatus 2 (Paddle Apparatus), 900 mL dissolution volume, 0.1 N hydrochloric acid dissolution medium, and paddle rotation of 50 RPM; and
at least about 80% of the acalabrutinib maleate is dissolved within about 30 minutes as determined in an in vitro dissolution test conducted using a USP Dissolution Apparatus 2 (Paddle Apparatus), 900 mL dissolution volume, 5 mM phosphate pH 6.8 dissolution medium, and paddle rotation of 75 RPM.

In another aspect, the dosage form satisfies the following conditions:
at least about 80% of the acalabrutinib maleate is dissolved within about 15 minutes as determined in an in vitro dissolution test conducted using a USP Dissolution Apparatus 2 (Paddle Apparatus), 900 mL dissolution volume, 0.1 N hydrochloric acid dissolution medium, and paddle rotation of 50 RPM; and
at least about 80% of the acalabrutinib maleate is dissolved within about 20 minutes as determined in an in vitro dissolution test conducted using a USP Dissolution Apparatus 2 (Paddle Apparatus), 900 mL dissolution volume, 5 mM phosphate pH 6.8 dissolution medium, and paddle rotation of 75 RPM.

In some embodiments, the solid pharmaceutical dosage forms of the present disclosure comprise about 75 mg to about 125 mg of acalabrutinib maleate (free base equivalent weight). In one aspect, the dosage forms comprise about 75 mg to about 100 mg of acalabrutinib maleate (free base equivalent weight). In another aspect, the dosage forms comprise about 75 mg to about 80 mg of acalabrutinib maleate (free base equivalent weight). In another aspect, the dosage forms comprise about 80 mg to about 85 mg of acalabrutinib maleate (free base equivalent weight). In another aspect, the dosage forms comprise about 85 mg to about 90 mg of acalabrutinib maleate (free base equivalent weight). In another aspect, the dosage forms comprise about 90 mg to about 95 mg of acalabrutinib maleate (free base equivalent weight). In another aspect, the dosage forms comprise about 95 mg to about 100 mg of acalabrutinib maleate (free base equivalent weight). In another aspect, the dosage forms comprise about 75 mg of acalabrutinib maleate (free base equivalent weight). In another aspect, the dosage forms comprise about 80 mg of acalabrutinib maleate (free base equivalent weight). In another aspect, the dosage forms comprise about 85 mg of acalabrutinib maleate (free base equivalent weight). In another aspect, the dosage forms comprise about 90 mg of acalabrutinib maleate (free base equivalent weight). In another aspect, the dosage forms comprise about 95 mg of acalabrutinib maleate (free base equivalent weight). In another aspect, the dosage forms comprise about 100 mg of acalabrutinib maleate (free base equivalent weight).

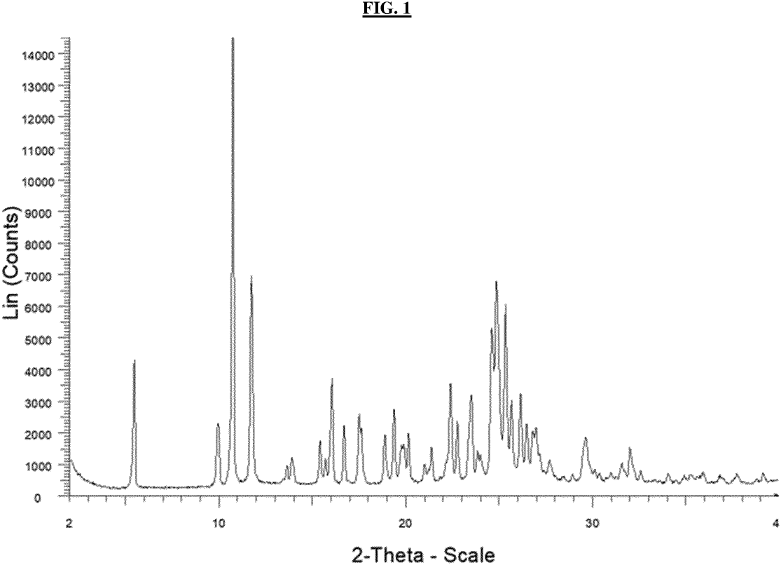

In some embodiments, the acalabrutinib maleate is acalabrutinib maleate monohydrate. In one aspect, the acalabrutinib maleate monohydrate is crystalline acalabrutinib maleate monohydrate. In another aspect, the crystalline acalabrutinib maleate is crystalline acalabrutinib maleate monohydrate Form A having an X-ray powder diffraction pattern comprising one or more peaks selected from the group consisting of an X-ray powder diffraction pattern with at least five peaks selected from the group consisting of 5.3, 9.8, 10.6, 11.6, 13.5, 13.8, 13.9, 14.3, 15.3, 15.6, 15.8, 15.9, 16.6, 17.4, 17.5, 18.7, 19.3, 19.6, 19.8, 20.0, 20.9, 21.3, 22.1, 22.3, 22.7, 23.2, 23.4, 23.7, 23.9, 24.5, 24.8, 25.2, 25.6, 26.1, 26.4, 26.7, 26.9, 27.1, 27.6, 28.8, 29.5, 30.0, 30.3, 30.9, 31.5, 31.9, 32.5, 34.0, and 35.1, with peak positions measured in °2θ ± 0.2 °2θ. In another aspect, the crystalline acalabrutinib maleate monohydrate Form A has an X-ray powder diffraction pattern comprising peaks at 5.3, 9.8, 10.6, 11.6, and 19.3 °2θ ± 0.2 °2θ. In another aspect, the X-ray powder diffraction pattern is substantially in agreement with the X-ray powder diffraction pattern of FIG. 1. In another aspect, the X-ray powder diffraction pattern of any of the foregoing embodiments is measured in transmission mode. In another aspect, the X-ray powder diffraction pattern of any of the foregoing embodiments is measured in reflection mode. In another aspect, the crystalline acalabrutinib maleate monohydrate of any of the foregoing embodiments has a stoichiometry relative to acalabrutinib that is approximately equivalent to a monohydrate. International Publication No. WO2017/002095 describes the applicable X-ray powder diffraction measurement conditions.

In some embodiments, the dosage form comprises acalabrutinib maleate wherein the acalabrutinib maleate has a crystalline purity of at least about 80% by weight of the acalabrutinib present in the dosage form. In one aspect, the crystalline purity is at least about 85% by weight. In another aspect, the crystalline purity is at least about 90% by weight. In another aspect, the crystalline purity is at least about 95% by weight. In another aspect, the crystalline purity is at least about 98% by weight. In another aspect, the crystalline purity is at least about 99% by weight. In another aspect, the acalabrutinib maleate is acalabrutinib maleate monohydrate. In another aspect, the acalabrutinib maleate is acalabrutinib maleate monohydrate Form A.

In some embodiments, the dosage form comprises acalabrutinib maleate wherein the acalabrutinib maleate has a crystalline purity of at least about 95% by weight of the acalabrutinib present in the dosage form. In one aspect, the acalabrutinib maleate is acalabrutinib maleate monohydrate. In another aspect, the acalabrutinib maleate is acalabrutinib maleate monohydrate Form A. In another aspect, the crystalline purity is at least about 96% by weight. In another aspect, the crystalline purity is at least about 97% by weight. In another aspect, the crystalline purity is at least about 98% by weight. In another aspect, the crystalline purity is at least about 99% by weight. In further aspects, the acalabrutinib maleate has a crystalline purity of at least about 95% by weight of the acalabrutinib present in the dosage form and comprises less than about 2% by weight of the impurity (2Z)-4-[(2S)-2-{8-amino-1-[4-(2-pyridinylcarbamoyl)phenyl]imidazo[1,5-a]pyrazin-3-yl}-1-pyrrolidinyl]-4-oxo-2-butenoic acid which has the chemical structure shown below:

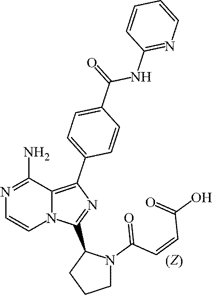

In another aspect, the acalabrutinib maleate comprises less than about 1.5% by weight of the impurity. In another aspect, the acalabrutinib maleate comprises less than about 1% by weight of the impurity. In another aspect, the acalabrutinib maleate comprises less than about 0.5% by weight of the impurity. In another aspect, the acalabrutinib maleate is substantially free of the impurity.

Selection of a salt of a compound rather than the free form of the compound does not necessarily improve solubility and uptake of the compound in the gastrointestinal tract to the extent desired. Further, the salts of a compound can differ significantly in physical and other properties that impact whether the salt is suitable for use in a pharmaceutical dosage form. For example, rapid conversion of a salt to a relatively insoluble free form in the acidic environment of the stomach as well as in the pH 6 to pH 7.5 environment of the intestine can result in some portion of the free form precipitating. Such precipitation of the free form results in a smaller amount of the administered dose available to be taken up in the gastrointestinal tract which results in a lower overall bioavailability of the compound. Surface properties (e.g., affecting wettability) and particle size (e.g., affecting the dissolution rate) are also among the factors that can affect the performance of the salt selected for the dosage form.

For example, the citrate, fumarate, gentisate, napadisylate, nitrate, oxalate, phosphate, sulfate, and L-tartrate salts of acalabrutinib were all determined to be unsuitable for use in the solid pharmaceutical dosage forms of the present disclosure. The citrate, fumarate, gentisate, and L-tartrate salts were eliminated from consideration based on their pKₐ values and/or evidence of complex solid-state behaviour. For example, the napadisylate salt had crystallinity issues. The nitrate salt could not be suitably manufactured at scale and generally is not favoured for use in pharmaceutical products. The oxalate, phosphate, and sulfate salts exhibited complex hydrate behaviour and were considered unsuitable for commercial manufacture.

In fact, the initial acalabrutinib maleate salt samples tested were deemed unlikely to achieve the solubility and dissolution rate required to overcome the limitations of the acalabrutinib free base in patients with elevated stomach pH. Further, although crystalline acalabrutinib maleate monohydrate Form A is thermodynamically stable under ambient conditions, it also exhibits solid-state properties that were initially believed to present challenges in the manufacture of commercial supplies of drug product.

In some embodiments, the present disclosure relates to solid pharmaceutical dosage forms wherein the dissolution rate of the acalabrutinib maleate in the in vitro dissolution test conducted using a USP Dissolution Apparatus 2 (Paddle Apparatus), 900 mL dissolution volume, 5 mM phosphate pH 6.8 dissolution medium, and paddle rotation of 75 RPM, does not decrease by more than 20% from its initial dissolution rate after storage of the dosage form in appropriate packaging for six months at 40° C. and 75% relative humidity. In one aspect, the dissolution rate does not decrease by more than 10% from its initial dissolution rate after storage of the dosage form in appropriate packaging for six months at 40° C. and 75% relative humidity. In one aspect, the dissolution rate does not decrease by more than 15% from its initial dissolution rate after storage of the dosage form in appropriate packaging for six months at 40° C. and 75% relative humidity. In another aspect, the dissolution rate does not decrease by more than 5% from its initial dissolution rate after storage of the dosage form in appropriate packaging for six months at 40° C. and 75% relative humidity. In another aspect, the dissolution rate does not decrease by more than 3% from its initial dissolution rate after storage of the dosage form in appropriate packaging for six months at 40° C. and 75% relative humidity. In another aspect, the dissolution rate does not decrease by more than 2% from its initial dissolution rate after storage of the dosage form in appropriate packaging for six months at 40° C. and 75% relative humidity. In another aspect, the dissolution rate does not decrease by more than 1% from its initial dissolution rate after storage of the dosage form in appropriate packaging for six months at 40° C. and 75% relative humidity. In one aspect, the packaging is a blister package such as an aluminum blister. In another aspect, the packaging is a sealed HDPE bottle with dessicant.

In some embodiments, the present disclosure relates to solid pharmaceutical dosage forms wherein no more than about 5% (w/w) of the acalabrutinib maleate present in the dosage form degrades after storage of the dosage form in appropriate packaging for six months at 40° C. and 75% relative humidity. In one aspect, no more than about 3% (w/w) of the acalabrutinib maleate present in the dosage form degrades after storage of the dosage form in appropriate packaging for six months at 40° C. and 75% relative humidity. In another aspect, no more than about 2% (w/w) of the acalabrutinib maleate present in the dosage form degrades after storage of the dosage form in appropriate packaging for six months at 40° C. and 75% relative humidity. In another aspect, no more than about 1% (w/w) of the acalabrutinib maleate present in the dosage form degrades after storage of the dosage form in a blister pack for six months at 40° C. and 75% relative humidity. In another aspect, no more than about 0.5% (w/w) of the acalabrutinib maleate present in the dosage form degrades after storage of the dosage form in appropriate packaging for six months at 40° C. and 75% relative humidity. In one aspect, the packaging is a blister package such as an aluminum blister. In another aspect, the packaging is a sealed HDPE bottle with dessicant. In another aspect, degradation of the acalabrutinib maleate is analyzed using high-performance liquid chromatography.

In some embodiments, the present disclosure relates to solid pharmaceutical dosage forms wherein the dosage form is substantially bioequivalent to a 100 mg Calquence® capsule (the composition of which corresponds to the contents of reference capsule C4 in Table 6 of Example 4) when orally administered to a fasting human subject who has not been administered a gastric acid reducing agent. In one aspect, the dosage form, when orally administered to a fasting human subject who has not been administered a gastric acid reducing agent, has a confidence interval of the relative mean Cₘₐₓ, AUC₍₀₋ₜ₎, and AUC_{(0-∞)} of the dosage form relative to the 100 mg Calquence® capsule for plasma acalabrutinib that is within 80% to 125%. In another aspect, the dosage form, when orally administered to a fasting human subject who has not been administered a gastric acid reducing agent, has a confidence interval of the relative mean Cₘₐₓ, AUC₍₀₋ₜ₎, and AUC_{(0-∞)} of the dosage form relative to the 100 mg Calquence® capsule for plasma acalabrutinib and its active metabolite ACP-5862 (i.e., 4-[8-Amino-3-[4-(but-2-ynoylamino)butanoyl]imidazo[1,5-a]pyrazin-1-yl]-N-pyridin-2-ylbenzamide) that are within 80% to 125%.

In some embodiments, the present disclosure relates to solid pharmaceutical dosage forms wherein the dosage form, when administered twice daily to a population of fasting human subjects, satisfies one or more of the following pharmacokinetic conditions for acalabrutinib:
the average Cₘₐₓ value in the population of human subjects is from about 400 ng/mL to about 900 ng/mL;
the average AUC₍₀₋₂₄₎ value in the population of human subjects is from about 350 ng•hr/mL to about 1900 ng•hr/mL; and/or
the average AUC_{(0-∞)} value in the population of human subjects is from about 350 ng•hr/mL to about 1900 ng•hr/mL.
In one aspect, the dosage form is co-administered to the population of human subjects with a gastric acid reducing agent.

In some embodiments the present disclosure relates to solid pharmaceutical dosage forms wherein the dosage form, when administered twice daily (BID) to a human subject, provides a median steady state Bruton tyrosine kinase occupancy of at least about 90% in peripheral blood mononuclear cells. In one aspect, the dosage form, when administered twice daily to a human subject, provides a median steady state Bruton tyrosine kinase occupancy of at least about 95% in peripheral blood mononuclear cells. In another aspect, the dosage form is co-administered to the population of human subjects with a gastric acid reducing agent.

In some embodiments, the present disclosure relates to solid pharmaceutical dosage forms wherein the acalabrutinib maleate is present in an amount of about 15% to about 55% by weight (free base equivalent weight) of the dosage form. In one aspect, the acalabrutinib maleate is present in an amount of about 25% to about 50% by weight of the dosage form. In another aspect, the acalabrutinib maleate is present in an amount of about 25% to about 45% by weight of the dosage form. In another aspect, the acalabrutinib maleate monohydrate is present in an amount of about 25% to about 40% by weight of the dosage form.

In some embodiments, the present disclosure relates to solid pharmaceutical dosage forms wherein the at least one pharmaceutically acceptable excipient is selected from at least one diluent, at least one disintegrant, and at least one lubricant. In one aspect, the at least one pharmaceutically acceptable excipient comprises at least one diluent. In another aspect, the at least one pharmaceutically acceptable excipient comprises at least one disintegrant. In another aspect, the at least one pharmaceutically acceptable excipient comprises at least one diluent and at least one disintegrant. In another aspect, the at least one pharmaceutically acceptable excipient comprises at least one diluent, at least one disintegrant, and at least one lubricant. Excipient interaction(s) in the dosage form potentially can impact the suitability of excipient combinations in the dosage forms of the present disclosure. Accordingly, the excipient combinations selected preferably do not materially affect the suitability of the dosage form for pharmacological use.

In some embodiments, the present disclosure relates to solid pharmaceutical dosage forms wherein the dosage comprises at least one diluent wherein the at least one diluent is present in an amount from about 10% to about 70% by weight of the dosage form. In one aspect, the at least one diluent is present in an amount from about 20% to about 70% by weight of the dosage form. In another aspect, the at least one diluent is present in an amount from about 30% to about 70% by weight of the dosage form. In another aspect, the at least one diluent is present in an amount from about 40% to about 70% by weight of the dosage form. In another aspect, the weight ratio of acalabrutinib maleate to the at least one diluent is from about 1:3 to about 2:1. In another aspect, the weight ratio of acalabrutinib maleate monohydrate to the at least one diluent is from about 1:1 to about 1:2.

When present, the diluent(s) selected preferably does not affect the stability of the primary amine moiety of acalabrutinib. In one aspect, the diluent is not susceptible to reacting with the primary amine moiety in a Maillard reaction. For example, the diluent is not a reducing sugar such as lactose. In addition, the diluent(s) preferably does not comprise a maleic acid scavenging agent such as a metal salt. In one aspect, the diluent(s) does not comprise dibasic calcium phosphate anhydrous. Acceptable diluents include, for example, sugar alcohols (such as mannitol, sorbitol, maltitol, and xylitol), hydrolysed starches, partially pre-gelatinised starches and celluloses (such as microcrystalline cellulose and silicified microcrystalline cellulose), and combinations thereof (such as a combination comprising mannitol and starch).

In some embodiments, the at least one diluent comprises a plastic diluent and a brittle diluent. A plastic diluent, such as microcrystalline cellulose, is one that undergoes irreversible deformation after exceeding the yield point during compression causing the particles to undergo viscous flow and stay deformed after removal of the compression force. A brittle diluent, such as mannitol, is one that undergoes fragmentation during compression, creating new surfaces for particle bonding. In one aspect, the dosage form comprises a plastic diluent and a brittle diluent in a total amount from about 10% to about 70% by weight of the dosage form; wherein the plastic diluent is present in an amount from about 0% to about 70% by weight of the dosage form; and the brittle diluent is present in an amount from about 0% to about 50% by weight of the dosage form. When the dosage form is a tablet, the ratio of plastic diluent to brittle diluent selected can impact the tensile strength of the tablet. An excess of plastic diluent can weaken the tensile strength of the tablet. In one aspect, the w/w ratio of plastic diluent to brittle diluent in the dosage form is from about 0:100 to about 60:40. In another aspect, the w/w ratio of plastic diluent to brittle diluent in the dosage form is from about 0:100 to about 60:40 wherein the dosage form is a tablet having a tensile strength of at least 2.0 MPa.

In some embodiments, the at least one diluent comprises mannitol. In one aspect, the mannitol is present in an amount from about 10% to about 70% by weight of the dosage form.

In some embodiments, the at least one diluent comprises microcrystalline cellulose. In one aspect, the microcrystalline cellulose is present in an amount from about 5% to about 50% by weight of the dosage form.

In some embodiments, the at least one diluent comprises mannitol and microcrystalline cellulose. In one aspect, the mannitol is present in an amount from about 0% to about 70% by weight of the dosage form; wherein the microcrystalline cellulose is present in an amount from about 0% to about 50% by weight of the dosage form; and the total amount of mannitol and microcrystalline cellulose is from about 10% to about 70% by weight of the dosage form. In another aspect, the w/w ratio of mannitol to microcrystalline cellulose is from about 0:100 to about 60:40. In another aspect, the w/w ratio of mannitol to microcrystalline cellulose in the dosage form is from about 0:100 to about 60:40 wherein the dosage form is a tablet having a tensile strength of at least 2.0 MPa.

In some embodiments, the present disclosure relates to solid pharmaceutical dosage forms wherein the dosage comprises at least one disintegrant and the at least one disintegrant is present in an amount from about 0.5% to about 15% by weight of the tablet. In one aspect, the at least one disintegrant is present in an amount from about 1% to about 10% by weight of the tablet. In another aspect, the at least one disintegrant is present in an amount from about 2% to about 8% by weight of the tablet. In another aspect, the at least one disintegrant is present in an amount from about 3% to about 7% by weight of the tablet. In another aspect, the weight ratio of acalabrutinib maleate (free base equivalent weight) to the at least one disintegrant is from about 2:1 to about 15:1. In another aspect, the weight ratio of acalabrutinib maleate to the at least one disintegrant is from about 4:1 to about 10:1.

When present, the disintegrant(s) selected preferably does not comprise an ionic disintegrant. In one aspect, the at least one disintegrant does not comprise sodium starch glycolate and/or croscarmellose sodium. In one aspect, the at least one disintegrant does not comprise sodium starch glycolate. In another aspect, the at least one disintegrant does not comprise croscarmellose sodium. Acceptable disintegrants include, for example, hydroxypropyl cellulose, maize starch, microcrystalline cellulose, crospovidone, and combinations thereof. In one aspect, the at least one disintegrant comprises hydroxypropyl cellulose. In another aspect, the at least one disintegrant comprises low-substituted hydroxypropyl cellulose.

In some embodiments, the present disclosure relates to solid pharmaceutical dosage forms wherein the dosage comprises at least one lubricant and the at least one lubricant is present in an amount from about 0.25% to about 4% by weight of the dosage form. In one aspect, the at least one lubricant is present in an amount from about 1% to about 4% by weight of the dosage form. In another aspect, the at least one lubricant is present in an amount from about 1.5% to about 3.5% by weight of the dosage form. In another aspect, the at least one lubricant is present in an amount from about 2% to about 3% by weight of the dosage form. In another aspect, the weight ratio of acalabrutinib maleate (free base equivalent weight) to the at least one lubricant is from about 20:1 to about 12:1. In another aspect, the weight ratio of acalabrutinib maleate to the at least one lubricant is from about 18:1 to about 14:1.

Acceptable lubricants include, for example, sodium stearyl fumarate, stearic acid, myristic acid, palmitic acid, sugar esters (such as sorbitan monostearate and sucrose monopalmitate), and combinations thereof. In another aspect, the at least one lubricant comprises sodium stearyl fumarate. Magnesium stearate generally should be avoided as the lubricant(s) selected.

In some embodiments, the present disclosure relates to solid pharmaceutical dosage forms wherein the dosage form comprises:
acalabrutinib maleate in an amount from about 15% to about 55% by weight (free base equivalent weight) of the dosage form;
at least one diluent in an amount from about 10% to about 70% by weight of the dosage form;
at least one disintegrant in an amount from about 0.5% to about 15% by weight of the dosage form; and
at least one lubricant in an amount from about 0.25% to about 4% by weight of the dosage form; and
wherein the sum of the individual amounts equals 100% of the total weight of the dosage form.
In one aspect, the dosage form consists essentially of the above-described components. In further aspects, the acalabrutinib maleate is present as acalabrutinib maleate monohydrate.

In some embodiments, the present disclosure relates to solid pharmaceutical dosage forms wherein the dosage form comprises:
acalabrutinib maleate monohydrate in an amount from about 20% to about 50% by weight (free base equivalent weight) of the dosage form;
at least one diluent in an amount from about 20% to about 70% by weight of the dosage form;
at least one disintegrant in an amount from about 1% to about 10% by weight of the dosage form; and
at least one lubricant in an amount from about 1% to about 4% by weight of the dosage form; and
wherein the sum of the individual amounts equals 100% of the total weight of the dosage form.
In one aspect, the dosage form consists essentially of the above-described components. In further aspects, the acalabrutinib maleate is present as acalabrutinib maleate monohydrate.

In some embodiments, the present disclosure relates to solid pharmaceutical dosage forms wherein the dosage form comprises:
acalabrutinib maleate in an amount from about 25% to about 50% by weight (free base equivalent weight) of the dosage form;
at least one diluent in an amount from about 30% to about 70% by weight of the dosage form;
at least one disintegrant in an amount from about 2% to about 8% by weight of the dosage form; and
at least one lubricant in an amount from about 1.5% to about 3.5% by weight of the dosage form; and
wherein the sum of the individual amounts equals 100% of the total weight of the dosage form.
In one aspect, the dosage form consists essentially of the above-described components. In further aspects, the acalabrutinib maleate is present as acalabrutinib maleate monohydrate.

In some embodiments, the present disclosure relates to solid pharmaceutical dosage forms wherein the dosage form comprises:
acalabrutinib maleate in an amount from about 25% to about 40% by weight (free base equivalent weight) of the dosage form;
at least one diluent in an amount from about 40% to about 70% by weight of the dosage form;
at least one disintegrant in an amount from about 3% to about 7% by weight of the dosage form; and
at least one lubricant in an amount from about 2% to about 3% by weight of the dosage form; and
wherein the sum of the individual amounts equals 100% of the total weight of the dosage form.
In one aspect, the dosage form consists essentially of the above-described components. In further aspects, the acalabrutinib maleate is present as acalabrutinib maleate monohydrate.

In some embodiments, the present disclosure relates to solid pharmaceutical dosage forms wherein the dosage form comprises:
acalabrutinib maleate in an amount from about 30% to about 35% by weight (free base equivalent weight) of the dosage form; and
mannitol in an amount from about 30% to about 35% by weight of the dosage form;
microcrystalline cellulose in an amount from about 25% to about 30% by weight of the dosage form;
hydroxypropyl cellulose in an amount from about 3% to about 7% by weight of the dosage form; and
sodium stearyl fumarate in an amount from about 1% to about 4% by weight of the dosage form; and
wherein the sum of the individual amounts equals 100% of the total weight of the dosage form.
In one aspect, the dosage form consists essentially of the above-described components. In further aspects, the acalabrutinib maleate is present as acalabrutinib maleate monohydrate.

In some embodiments, the present disclosure relates to solid pharmaceutical dosage forms wherein the acalabrutinib maleate has a D_{(v,} _{0.9)} value below about 500 microns. In one aspect, the acalabrutinib maleate has a D_{(v,} _{0.9)} value below about 450 microns. In another aspect, the acalabrutinib maleate has a D_{(v,} _{0.9)} value below about 400 microns. In another aspect, the acalabrutinib maleate has a D_{(v,} _{0.9)} value below about 350 microns. In another aspect, the acalabrutinib maleate has a D_{(v,} _{0.9)} value below about 300 microns. In another aspect, the acalabrutinib maleate has a D_{(v,} _{0.9)} value from about 20 microns to about 500 microns. In another aspect, the acalabrutinib maleate has a D_{(v,} _{0.9)} value from about 50 microns to about 450 microns. In another aspect, the acalabrutinib maleate has a D_{(v,} _{0.9)} value from about 75 microns to about 400 microns. In another aspect, the acalabrutinib maleate has a D_{(v,} _{0.9)} value from about 75 microns to about 350 microns. In another aspect, the acalabrutinib maleate has a D_{(v,} _{0.9)} value from about 100 microns to about 300 microns.

In some embodiments, the present disclosure relates to solid pharmaceutical dosage forms wherein the acalabrutinib maleate satisfies one or more of the following conditions: a D_{(v,} _{0.1)} value below about 20 microns, a D_{(v,} _{0.5)} value below about 145 microns, and a D_{(v,} _{0.9)} value below about 330 microns. In another aspect, the acalabrutinib maleate has a D_{(v,} _{0.5)} value below about 145 microns and a D_{(v,} _{0.9)} value below about 330 microns. In another aspect, the acalabrutinib maleate has a D_{(v,} _{0.1)} value below about 20 microns, a D_{(v,} _{0.5)} value below about 145 microns, and a D_{(v,} _{0.9)} value below about 330 microns.

In some embodiments, the present disclosure relates to solid pharmaceutical dosage forms wherein the dosage form is a capsule. In one aspect, the capsule is prepared by a process comprising roller compaction.

In some embodiments, the present disclosure relates to solid pharmaceutical dosage forms wherein the dosage form is a tablet. In one aspect, the dosage form is a film-coated tablet. In another aspect, the film coat is a stabilizing film coat. In another aspect, the tablet is prepared by a process comprising direct compression. In another aspect, the tablet is prepared by a process comprising roller compaction. In another aspect, the tablet is prepared by a process comprising wet granulation. In another aspect, the tablet has a tensile strength from about 1.5 MPa to about 5.0 MPa. In another aspect, the tablet has a tensile strength from about 2.0 MPa to about 4.0 MPa. In another aspect, the tablet tensile strength does not decrease by more than 10% from its initial tensile strength after storage of the tablet in appropriate packaging for six months at 40° C. and 75% relative humidity. In another aspect, the tablet tensile strength does not decrease by more than 8% from its initial tensile strength after storage of the tablet in appropriate packaging for six months at 40° C. and 75% relative humidity. In another aspect, the tablet tensile strength does not decrease by more than 5% from its initial tensile strength after storage of the tablet in appropriate packaging for six months at 40° C. and 75% relative humidity. In one aspect, the packaging is a blister package such as an aluminum blister. In another aspect, the packaging is a sealed HDPE bottle with dessicant.

In some embodiments, the tablet is a coated or uncoated tablet having a core weight less than about 600 mg. In another aspect, dosage form is a coated or uncoated tablet having a core weight from about 300 mg to about 500 mg. In another aspect, dosage form is a coated or uncoated tablet having a core weight from about 350 mg to about 450 mg. In another aspect, dosage form is a coated or uncoated tablet having a core weight of about 400 mg.

### III. Methods of Treatment

The present disclosure also relates to methods of treating a BTK-mediated condition in a subject, particularly a human subject suffering from or susceptible to the condition, comprising administering once or twice daily to the subject a solid pharmaceutical dosage form comprising acalabrutinib maleate as described in any of the embodiments of the disclosure. In one aspect, the solid pharmaceutical dosage form comprising acalabrutinib maleate is administered once daily. In another aspect, the solid pharmaceutical dosage form comprising acalabrutinib maleate is administered twice daily.

In one embodiment, the present disclosure relates to methods of treating a B-cell hematological malignancy in a subject, particularly a human subject suffering from or susceptible to the condition, comprising administering once or twice daily to the subject a solid pharmaceutical dosage form comprising acalabrutinib maleate as described in any of the embodiments of the disclosure. In one aspect, the solid pharmaceutical dosage form comprising acalabrutinib maleate is administered once daily. In another aspect, the solid pharmaceutical dosage form comprising acalabrutinib maleate is administered twice daily.

In some embodiments, the B-cell hematological malignancy is selected from the group consisting of non-Hodgkin’s lymphoma (NHL), Hodgkin’s lymphoma, mantle cell lymphoma (MCL), chronic lymphocytic leukemia (CLL), small lymphocytic leukemia (SLL), diffuse large B-cell lymphoma (DLBCL), follicular lymphoma (FL), B-cell acute lymphoblastic leukemia (B-ALL), Burkitt’s lymphoma, Waldenström’s macroglobulinemia (WM), multiple myeloma, myelodysplastic syndromes, and myelofibrosis.

In some embodiments, the B-cell hematological malignancy is non-Hodgkin’s lymphoma. In one aspect, the non-Hodgkin’s lymphoma is aggressive non-Hodgkin’s lymphoma. In another aspect, the non-Hodgkin’s lymphoma is indolent non-Hodgkin’s lymphoma.

In some embodiments, the B-cell hematological malignancy is Hodgkin’s lymphoma.

In some embodiments, the B-cell hematological malignancy is selected from the group consisting of mantle cell lymphoma, chronic lymphocytic leukemia, and small lymphocytic leukemia.

In some embodiments, the B-cell hematological malignancy is mantle cell lymphoma. In one aspect, the mantle cell lymphoma is mantle zone lymphoma. In another aspect, the mantle cell lymphoma is nodular mantle cell lymphoma. In another aspect, the mantle cell lymphoma is diffuse mantle cell lymphoma. In another aspect, the mantle cell lymphoma is blastoid mantle cell lymphoma.

In some embodiments, the B-cell hematological malignancy is chronic lymphocytic leukemia.

In some embodiments, the B-cell hematological malignancy is small lymphocytic leukemia.

In some embodiments, the B-cell hematological malignancy is diffuse large B-cell lymphoma. In one aspect, the diffuse large B-cell lymphoma is selected from the group consisting of de novo diffuse large B-cell lymphoma, relapsed/refractory diffuse large B-cell lymphoma, and transformed diffuse large B-cell lymphoma. In another aspect, the diffuse large B-cell lymphoma is de novo diffuse large B-cell lymphoma. In another aspect, the diffuse large B-cell lymphoma is relapsed/refractory diffuse large B-cell lymphoma. In another aspect, the diffuse large B-cell lymphoma is transformed diffuse large B-cell lymphoma. In another aspect, the transformed diffuse large B-cell lymphoma is Richter syndrome.

In some embodiments, the diffuse large B-cell lymphoma is selected from the group consisting of the germinal center B-cell diffuse large B-cell lymphoma and activated B-cell diffuse large B-cell lymphoma subtypes. In one aspect, the diffuse large B-cell lymphoma is relapsed/refractory germinal center B-cell diffuse large B-cell lymphoma. In another aspect, the diffuse large B-cell lymphoma is relapsed/refractory activated B-cell diffuse large B-cell lymphoma.

In some embodiments, the B-cell hematological malignancy is follicular lymphoma.

In some embodiments, the B-cell hematological malignancy is Waldenström’s macroglobulinemia.

In some embodiments, the B-cell hematological malignancy is B-cell acute lymphoblastic leukemia. In one aspect, the B-cell acute lymphoblastic leukemia is early pre-B-cell acute lymphoblastic leukemia. In another aspect, the B-cell acute lymphoblastic leukemia is pre-B-cell acute lymphoblastic leukemia. In another aspect, the B-cell acute lymphoblastic leukemia is mature B-cell acute lymphoblastic leukemia.

In some embodiments, the B-cell hematological malignancy is Burkitt’s lymphoma. In one aspect, the Burkitt’s lymphoma is sporadic Burkitt’s lymphoma. In another aspect, the Burkitt’s lymphoma is endemic Burkitt’s lymphoma. In another aspect, the Burkitt’s lymphoma is human immunodeficiency virus-associated Burkitt’s lymphoma.

Diagnosis of the specific B-cell malignancy from which a subject is suffering can be made in accordance with accepted clinical practice. See, for example, the 2016 classification guidelines established by the World Health Organization (WHO) for lymphoid neoplasms, or the National Comprehensive Cancer Network (NCCN) classification guidelines for non-Hodgkin lymphoma.

In some embodiments, the human subject has previously received at least one prior chemo-immunotherapy for the B-cell malignancy. In one aspect, the prior chemo-immunotherapy comprises treatment with cyclophosphamide, doxorubicin, vincristine, and prednisolone (CHOP) or with rituximab, cyclophosphamide, doxorubicin, vincristine, and prednisolone (R-CHOP). In another aspect, the prior chemo-immunotherapy comprises treatment with fludarabine, cyclophosphamide, and rituximab (FCR). In another aspect, the prior chemo-immunotherapy comprises treatment with rituximab and bendamustin (BR). In another aspect, the prior chemo-immunotherapy comprises treatment with chlorambucil and obinutuzumab.

In some embodiments, the human subject has previously received treatment with a BTK inhibitor other than acalabrutinib (such as ibrutinib or zanubrutinib).

In another embodiment, the present disclosure relates to use of the solid pharmaceutical dosage forms comprising acalabrutinib maleate as described in any of the embodiments of the disclosure for the treatment of B-cell malignancies.

In another embodiment, the present disclosure relates to use of the solid pharmaceutical dosage forms comprising acalabrutinib maleate as described in any of the embodiments of the disclosure in the manufacture of medicaments for the treatment of B-cell malignancies.

In some embodiments, the solid pharmaceutical dosage form comprising acalabrutinib maleate is co-administered to the subject together with a gastric acid reducing agent such as a proton pump inhibitor, an H2-receptor antagonist, or an antacid. In one aspect, the co-administration is simultaneous. In another aspect, the co-administration is sequential.

In some embodiments, the present disclosure relates to methods of improving the pharmacokinetics of orally administered acalabrutinib over a broader range of acidic stomach conditions in a subject suffering from or susceptible to a B-cell hematological malignancy comprising administering to the subject once (QD) or twice (BID) daily the solid pharmaceutical dosage form containing acalabrutinib maleate as described in any of the embodiments of the disclosure. In one aspect, the method improves and/or decreases the intra-and/or inter-subject variability of acalabrutinib bioavailability. In another aspect, the method reduces the intra- and/or inter-subject variability of acalabrutinib pharmacokinetics. In another aspect, the method improves and/or decreases the intra- and/or inter-subject variability of acalabrutinib Cₘₐₓ. In another aspect, the method improves and/or decreases the intra- and/or inter-subject variability of acalabrutinib Tₘₐₓ. In another embodiment, the improves and/or decreases the intra- and/or inter-subject variability of acalabrutinib AUC_{(0-∞)}.

In some embodiments, the present disclosure relates to methods of treating a human subject infected by SARS-CoV-2 and/or having coronavirus disease 2019 (COVID-19) comprising administering to the subject the solid pharmaceutical dosage form(s) containing acalabrutinib maleate as described in any of the embodiments of the disclosure.

In another embodiment, the present disclosure relates to use of the solid pharmaceutical dosage forms comprising acalabrutinib maleate as described in any of the embodiments of a human subject infected by SARS-CoV-2 and/or having coronavirus disease 2019 (COVID-19).

In another embodiment, the present disclosure relates to use of the solid pharmaceutical dosage forms comprising acalabrutinib maleate as described in any of the embodiments of the disclosure in the manufacture of medicaments for the treatment of a human subject infected by SARS-CoV-2 and/or having coronavirus disease 2019 (COVID-19).

The methods of the present disclosure also contemplate treatments comprising co-administering a solid pharmaceutical dosage form comprising acalabrutinib maleate as described in any of the embodiments of the disclosure with one or more additional therapeutic agents. Accordingly, the dosage forms of the present disclosure can be administered alone or in combination with one or more additional therapeutic agents. When administered in combination with one or more additional therapeutic agents, the additional therapeutic agent may be administered simultaneously with the acalabrutinib maleate dosage form of the present disclosure or sequentially with the acalabrutinib maleate dosage form of the present disclosure. In one aspect, the therapeutic agent is anti-CD20 antibody. In another aspect, anti-CD20 antibody is selected from the group consisting of rituximab, ocrelizumab, obinutuzumab, ofatumumab, ibritumomab tiuxetan, tositumomab, and ublituximab. In another aspect, the anti-CD20 antibody is selected from the group consisting of rituximab, obinutuzumab, and ofatumumab. In another aspect, the anti-CD20 antibody is rituximab. In another aspect, the anti-CD20 antibody is obinutuzumab. In another aspect, the anti-CD20 antibody is ofatumumab.

### IV. Kits

The present disclosure further relates, in part, to kits comprising one or more solid pharmaceutical dosage forms comprising acalabrutinib maleate as described in any of the embodiments of the disclosure. The kits optionally can comprise one or more additional therapeutic agents and/or instructions for using the kit. Suitable packaging and additional articles for use are known in the art and may be included in the kit. The kits can be provided, marketed and/or promoted to health providers, including physicians, nurses, pharmacists, formulary officials, and the like.

In some embodiments, the kit comprises a semi-permeable container containing one or more solid pharmaceutical dosage forms comprising acalabrutinib maleate. In one aspect, the semi-permeable container is a blister package.

In some embodiments, the kit comprises a substantially impermeable container containing one or more solid pharmaceutical dosage forms comprising acalabrutinib maleate. In one aspect, the impermeable container is an HDPE bottle with dessicant.

In some embodiments, the kit comprises a plurality of separate packages with each package containing a daily dose of the solid pharmaceutical dosage forms comprising acalabrutinib maleate (e.g., a package containing one or two of the solid dosage forms).

The kits described above are preferably for use in the treatment of the B-cell malignancies described in this specification. For example, in one aspect, the B-cell malignancy is non-Hodgkin lymphoma. In another aspect, the B-cell malignancy is mantle cell lymphoma. In another aspect, the B-cell malignancy is chronic lymphocytic leukemia. In another aspect, the B-cell malignancy is small lymphocytic leukemia. In another aspect, the B-cell malignancy is diffuse large B-cell lymphoma.

In another embodiment, the kits described above are for use in the treatment of a human subject infected by SARS-CoV-2 and/or having coronavirus disease 2019 (COVID-19).

### V. Methods of Preparation

The present disclosure also relates to methods for preparing the solid pharmaceutical dosage forms comprising acalabrutinib maleate described in this disclosure, including those methods described in the Examples below. In general, these dosage forms can be prepared using techniques such as, but not limited to, direct blending, dry granulation (roller compaction), wet granulation (high shear granulation), milling or sieving, drying (if wet granulation is used), compression, and, optionally, coating.

### VI. Product-By-Process

The present disclosure also relates to solid pharmaceutical dosage forms comprising acalabrutinib maleate prepared in accordance with any of the methods described in this disclosure, including the methods described in the Examples below.

### VII. Examples

### Example 1: Assessment of Acalabrutinib Salts

### 1. Dissolution Study

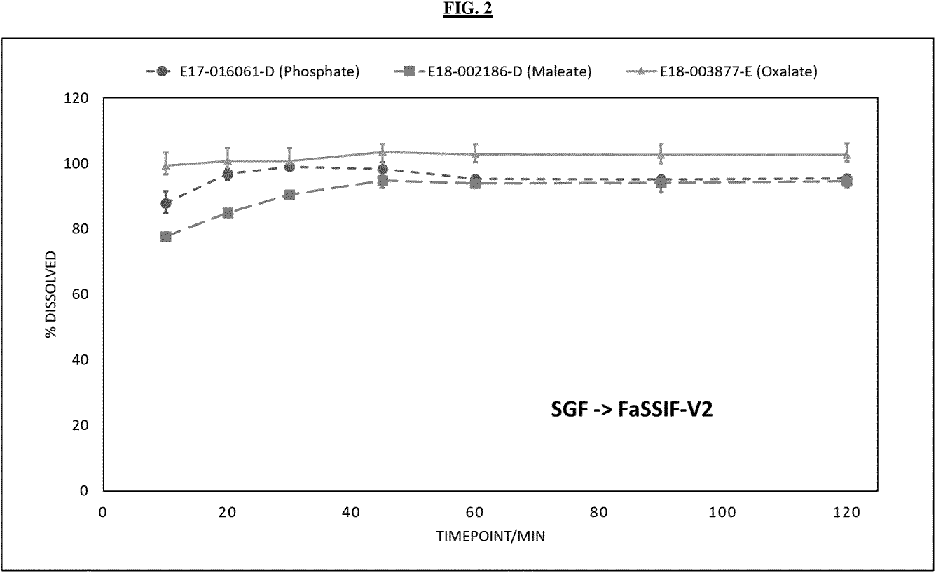

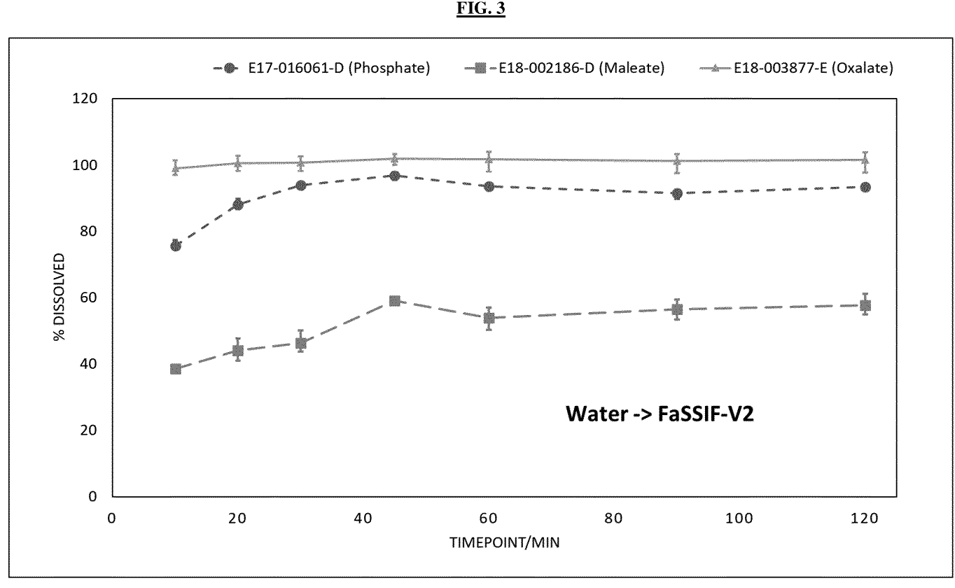

Phosphate, oxalate, and maleate salts of acalabrutinib were evaluated using a two-stage in vitro dissolution method known as a pH shift method. The initial medium was either deionized water or a simulated gastric fluid containing hydrochloric acid and sodium chloride and pH adjusted to 1.8. After the salts were in the initial medium for 30 minutes, the medium was then changed to a FaSSIF-V2 medium by addition of a double strength concentrate to give a final pH of 6.5. The FaSSIF-V2 medium contained a sodium phosphate buffer with sodium chloride, sodium taurocholate, and lecithin. The dissolution testing was conducted using USP dissolution apparatus 2 (paddle) operating at 50 RPM at 37 ± 0.5° C. in 250 mL of medium for first 30 minutes and then in 500 mL of medium post-shift. Samples from the dissolution medium were pulled from the aqueous phase at predetermined time points and assayed by HPLC. FIGS. 2 and 3 show the dissolution profiles of the three salts in the simulated gastric fluid/FaSSIF-V2 media and the deionized water/FaSSIF-V2 media, respectively. Although the three salts exhibited broadly similar performance in the low pH simulated gastric fluid medium, the maleate salt exhibited substantially reduced dissolution in the neutral water medium relative to the oxalate and the phosphate salts.

### 2. Physical Property Study

The physical properties of phosphate, oxalate, and maleate salts of acalabrutinib were investigated, including physical stability, crystallinity, and particle habit.

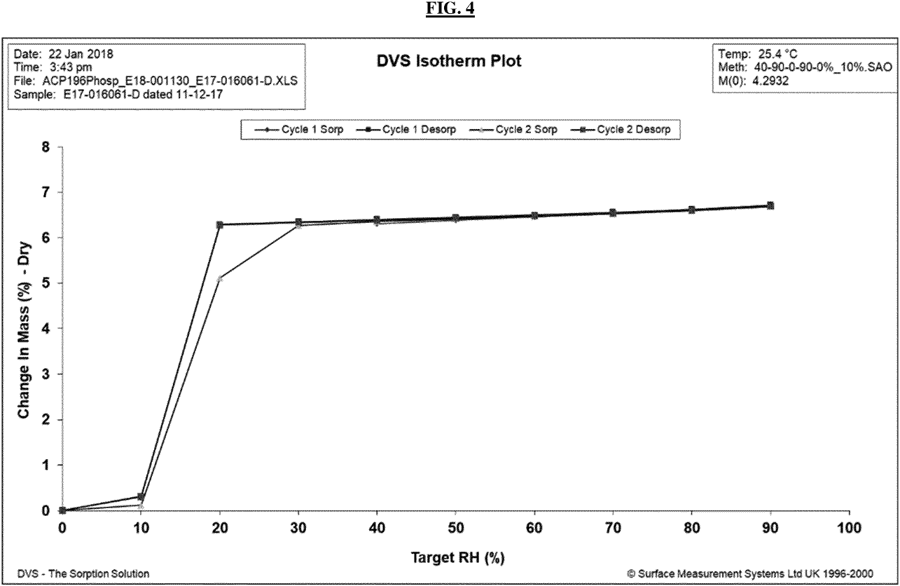

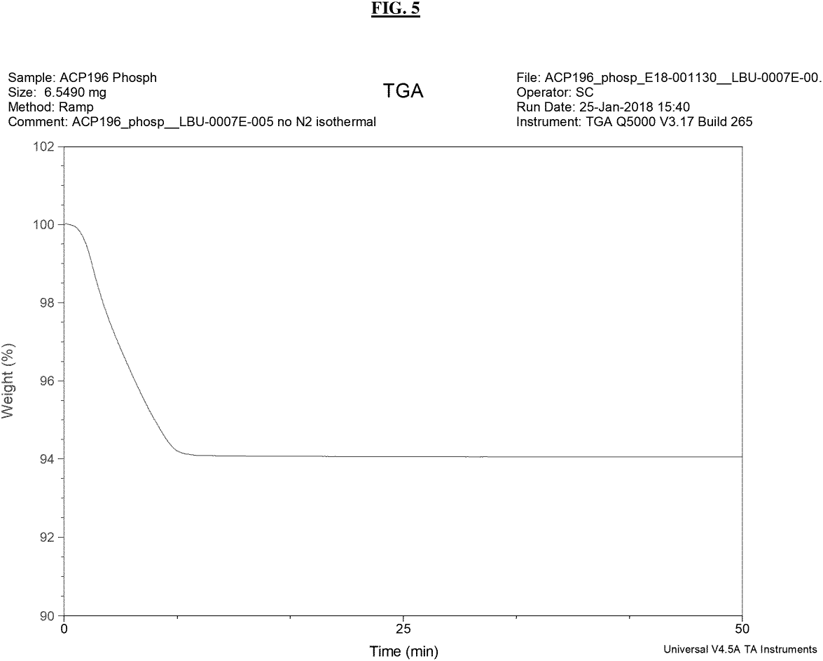

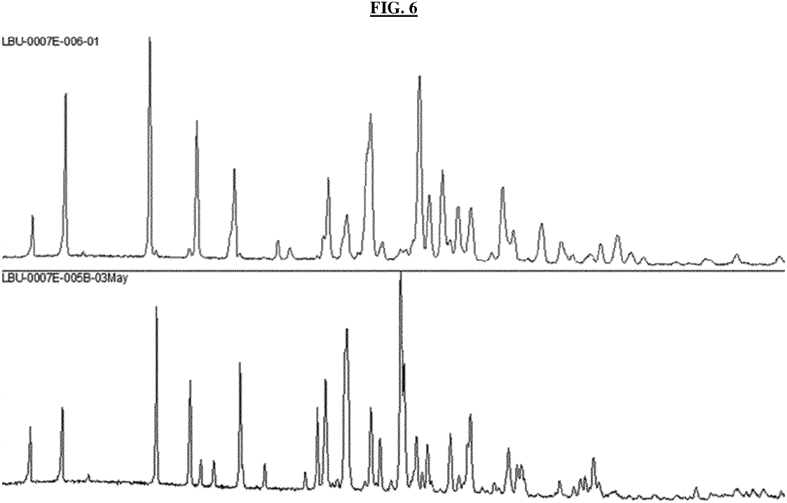

Solid-state analysis of the phosphate salt showed complex hydrate behaviour around ambient conditions where the solid would switch between hydrated forms with conversion of one crystalline form to a higher hydrate crystalline form at relative humidities (“RH”) above 20% RH as evidenced by the dynamic vapour sorption (“DVS”) plot in FIG. 4. Thermogravimetric analysis (“TGA”) indicated that the higher hydrate was physically unstable, rapidly dehydrating in less than 10 minutes under open pan, isothermal conditions at 40° C. as evidenced by FIG. 5. Standard TGA further indicated that the phosphate salt batches often were inhomogeneous in terms of water content, and therefore in terms of physical form. X-ray powder diffraction (“XRPD”) evidenced that both crystalline forms could be identified as shown by FIG. 6.

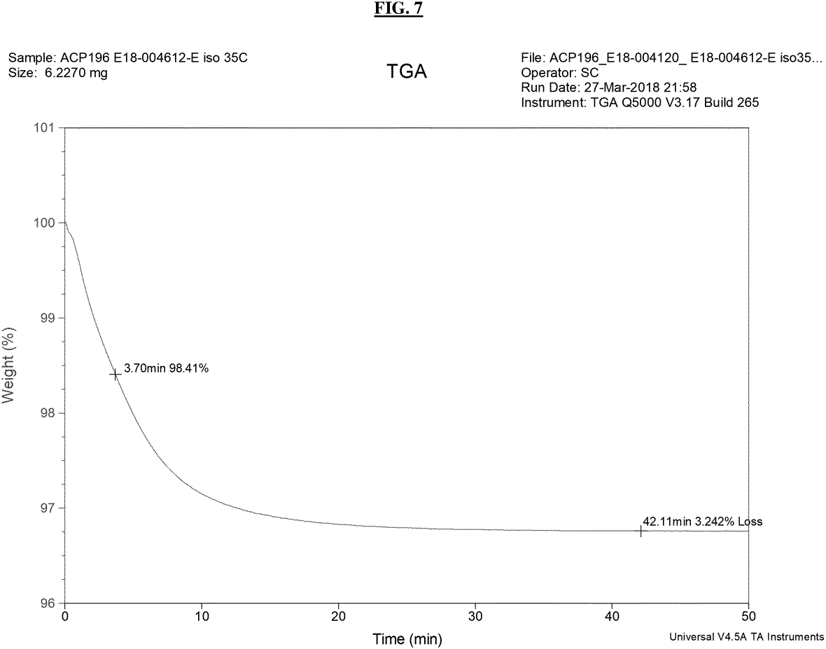

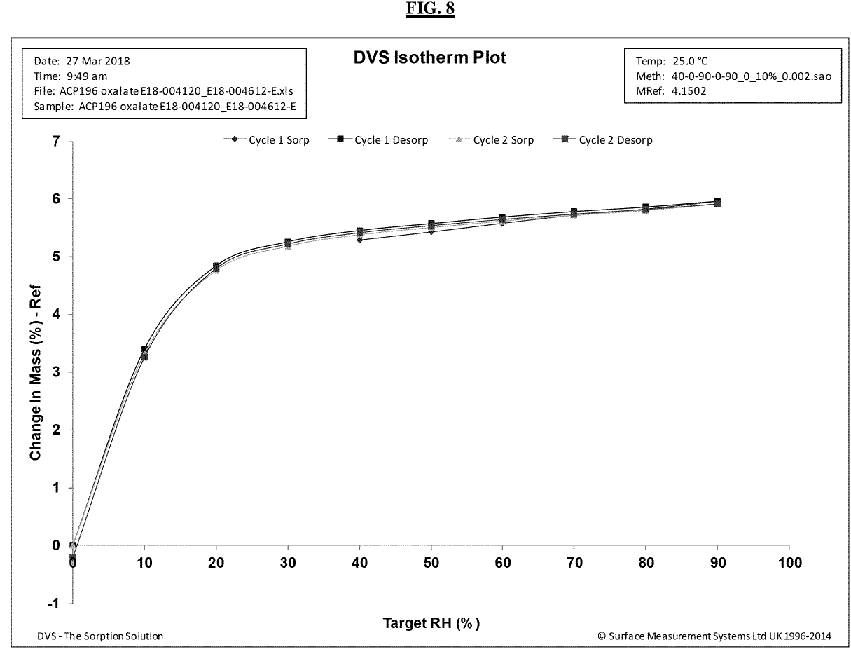

The oxalate salt also exhibited complex hydrate behavior. TGA indicated that the hydrate was very labile as evidenced by FIG. 7. Under isothermal TGA conditions at 35° C., the water loss exhibited a half-life of 4 minutes and an overall weight loss of 3.2% w/w. The water loss was consistent with approximately one mole of water per mole of oxalate salt. DVS showed the conversion of one crystalline form to a higher hydrate crystalline form at ambient humidities as evidenced by FIG. 8. When analysed by light microscopy, the oxalate salt evidenced a very fine needle habit.

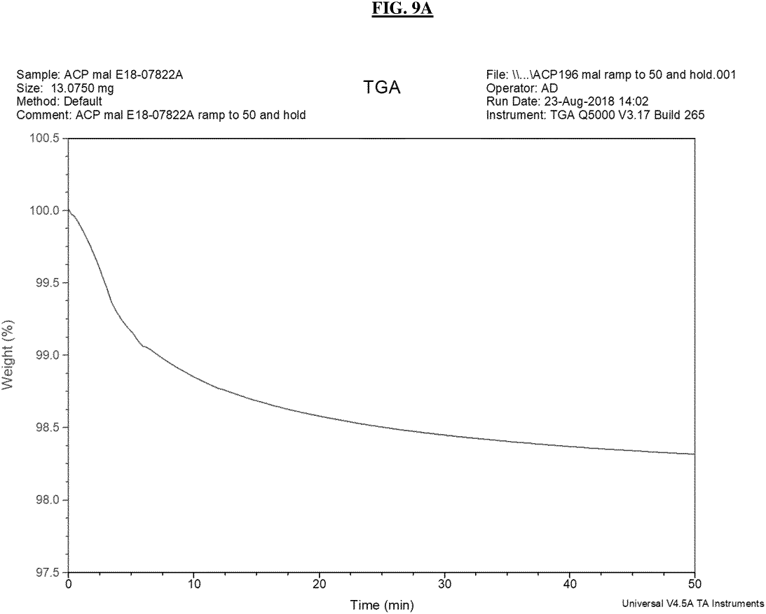

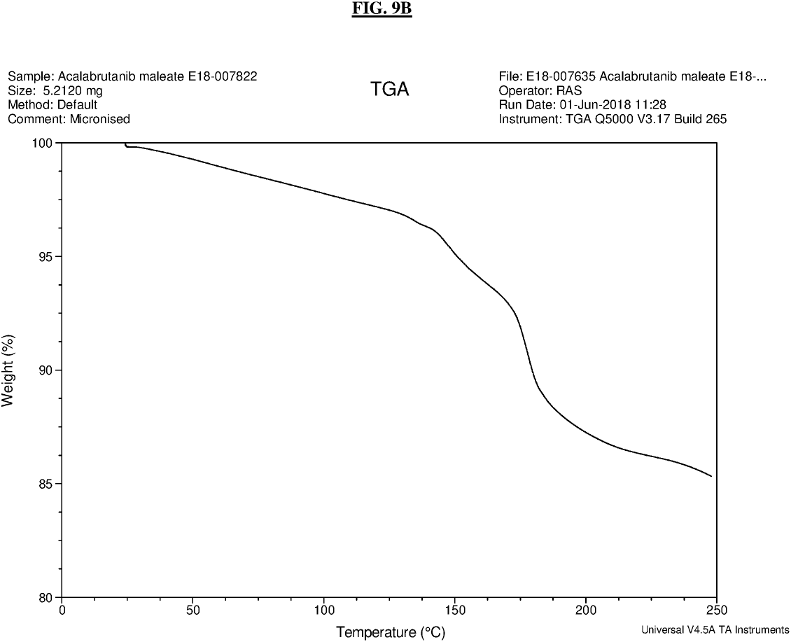

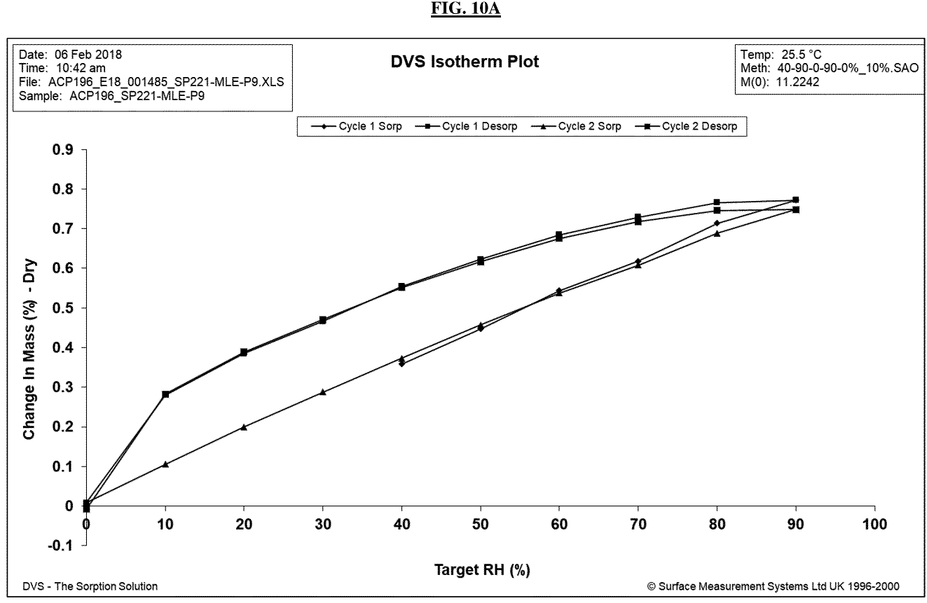

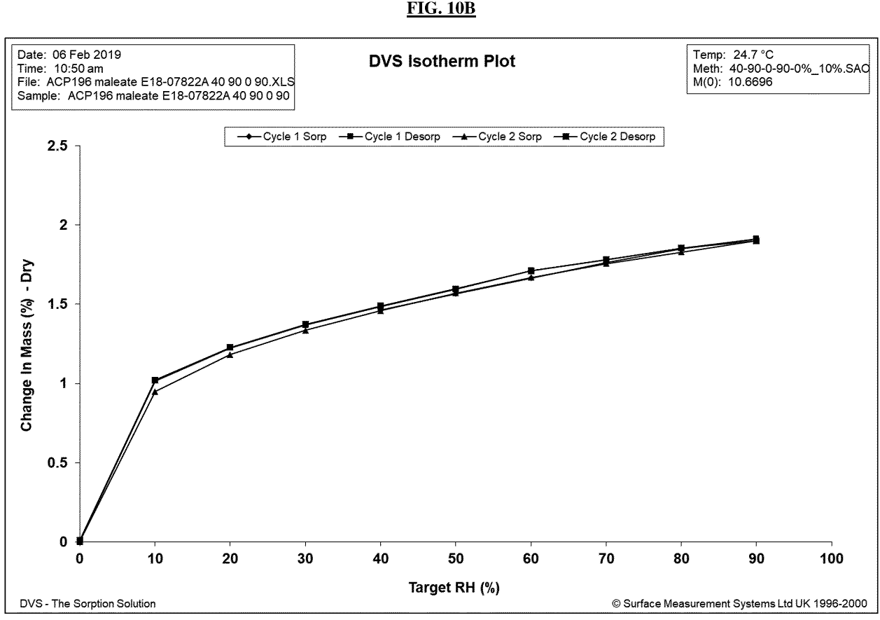

The maleate salt was isolated as a monohydrate. Although isothermal TGA at 50° C. indicated that the monohydrate dehydrates as shown by FIG. 9A, the dehydration rate was slower than that of the phosphate or oxalate salts at the lower temperatures of 40° C. and 35° C., respectively. FIG. 9B is a TGA plot carried out under an alternative set of conditions. The DVS plot of the maleate salt in FIG. 10A indicated that the change in the % w/w water across the humidity range was less than observed with the phosphate or oxalate salts. FIG. 10B is a DVS plot for a higher quality sample of the maleate salt. The crystal habit of the maleate salt was large and block-like.

Although the phosphate and oxalate salts exhibited substantially better dissolution in the neutral water medium than the maleate salt, the physical properties of the phosphate and oxalate salts presented greater challenges in developing a pharmaceutically acceptable formulation comprising an acalabrutinib salt.

### 3. Dissolution of Micronized Maleate Salt

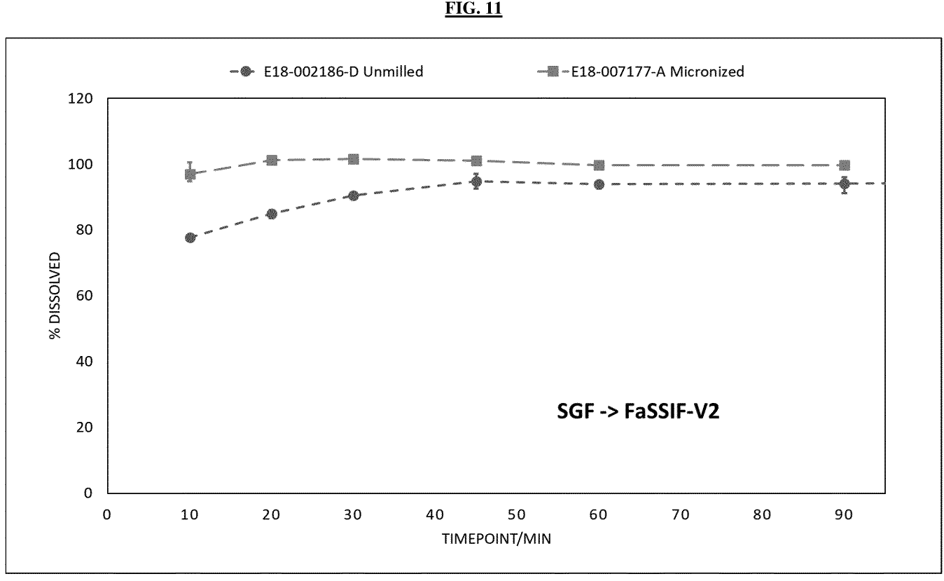

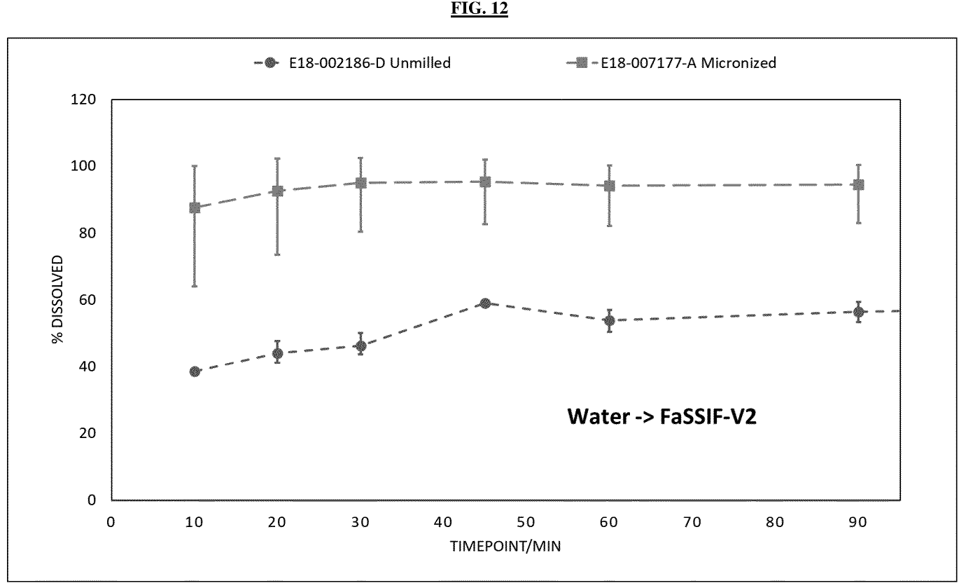

In view of the formulation challenges associated with the physical properties of the phosphate and oxalate salts, the maleate salt was re-tested in the previously described pH shift dissolution method after undergoing particle size reduction. The typical mean values of the D_{(v,} _{0.9)} particle size distribution for the micronized maleate salt batches and unmilled maleate salt batches tested typically were around 18 µm and around 446 µm, respectively. Using the same method conditions as previously described, a micronized sample of the maleate salt was tested and exhibited a dissolution profile that was significantly improved (and to greater extent than one skilled in the art would have expected) relative to the unmilled sample of the maleate salt. FIGS. 11 and 12 show the dissolution profiles of the micronized and unmilled maleate salts in the simulated gastric fluid/FaSSIF-V2 media and the deionized water/FaSSIF-V2 media, respectively.

### Example 2: Assessment of Acalabrutinib Maleate Solubility

The solubility of acalabrutinib maleate was measured in unbuffered media and found to be around 3 mg/mL at pH 4 with the pHₘₐₓ calculated to be at 4.11. It was further determined that in unbuffered media with a starting pH higher than pH 4 and up to about pH 11 acalabrutinib maleate buffers its surface pH to a value ranging between 3.8 to 5 and that the solubility of acalabrutinib maleate in unbuffered media from pH 4 to pH 11 remains around 3 mg/mL In contrast, the solubility of acalabrutinib free base in unbuffered media decreased to less than about 0.1 mg/mL as the pH approached pH 6.

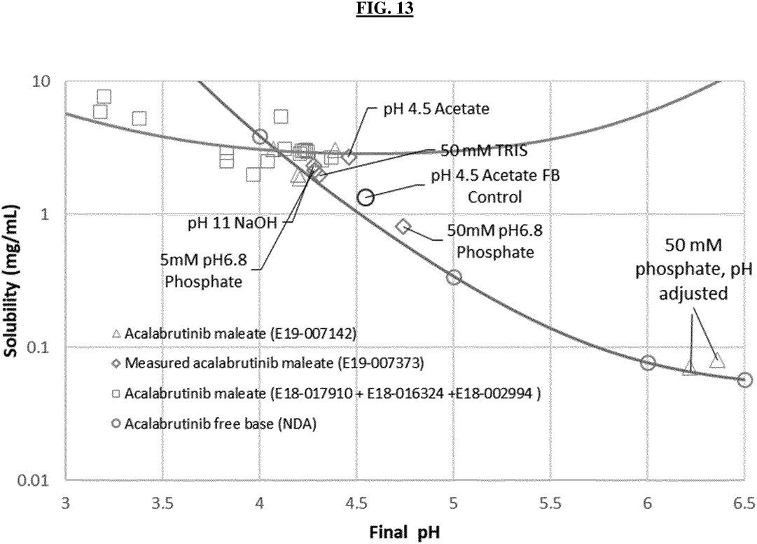

In addition, the solubility of acalabrutinib maleate was measured in buffered solutions representative of the media used for dissolution of acalabrutinib maleate tablets. It was found that the final pH was also influenced by the presence of acalabrutinib maleate and, depending on the buffer used, the acalabrutinib maleate was able to super-saturate compared to the free base at equivalent final pH or to exhibit solubility values close to that of the free base at an equivalent final pH. For example, acalabrutinib maleate in pH 4.5 acetate buffer supersaturated with a solubility significantly higher than that of the free base in pH 4.5. In phosphate buffer, the phosphate concentration and eventual pH adjustment controlled the final pH and the final solubility of acalabrutinib maleate, but the values observed in all conditions were close to that of the acalabrutinib free base at an equivalent final pH. FIG. 13 depicts the solubility versus final pH values for acalabrutinib maleate and acalabrutinib free base in a variety of buffered solutions.

### Example 3: Physicochemical Properties of Acalabrutinib Maleate Monohydrate

Selected physicochemical properties of acalabrutinib maleate monohydrate were determined and are reported in Table 2 below.

TABLE 2

| PROPERTY | VALUE |
|---|---|
| Structure | Acalabrutinib maleate monohydrate |
| Molecular weight (g/mol) | 465.51 (Free Base), 599.61 (Salt) |
| Physical form | Crystalline, Salt to Base ratio = 1.29 |
| pKa (s) | Maleic acid (1.83(A), 6.07(A)) + Free Base (3.54 (B), 5.77 (B) and 12.1 (A)) |
| LogP/LogD (with pH) | 2.0 |
| Melting point | Onset at 150° C. (thermal degradation following loss of water) |
| Intrinsic solubility | 3 mg/mL (@ pH 4 Salt) 48 ug/mL (@ pH 8 Free Base) |
| Solubility at pH 4 (mg/mL) | 3 |
| Intrinsic permeability (cm/s) | 5.4 10⁻⁴ |
| Efflux potential | In vitro Vₘₐₓ =79.6 10⁻⁶ µg.s⁻¹, Km = 5.97 µM |
| Fraction Unbound (fᵤ) | 2.6% |
| Density (g/mL) (Mw/Molar volume) | 1.359 g/mL (Salt) 1.36 g/mL (Free Base) |
| Diffusivity in water (x1E-9 m²/s) | 0.646 (calculated for Free Base at 37° C.) |
| Blood: Plasma ratio | 0.787 |

### Example 4: Acalabrutinib Maleate Tablets

Tablets comprising acalabrutinib maleate monohydrate and various excipients were prepared by either direct compression or roller compaction and are further described below. The direct compression tablets were uncoated and the roller-compacted tablets were film-coated. All tablets prepared contained a unit dose of approximately 100 mg equivalent weight of acalabrutinib maleate monohydrate.

### A. Direct Compression Tablets

Tablets having the compositions set forth in Tables 3 and 4 were prepared by direct compression. Prior to tablet compression, all components, except for the lubricant, were blended, then screened through a sieve, and then blended again. Screened lubricant was added to the blend, which was then lubricated by further blending. Tablets were compressed using a suitable tablet press and tooling appropriate for the target tablet compression weight. Where tablets required additional lubrication (i.e., where punch picking or sticking was observed), additional lubricant was applied externally to the tablet die.

TABLE 3

COMPOSITION OF DIRECT COMPRESSION TABLETS

| Component | T1 (g/batch) EB18-254510 | T2 (g/batch) EB18-396006 | T3 (g/batch) EB18-396008 | T4 (g/batch) EB18-432106 | T5 (g/batch) EB18-431408 |
|---|---|---|---|---|---|
| Acalabrutinib maleate | 10^{a} | 14.835^{b} | 14.835^{b} | 16.125^{b} | 16.125^{b} |
| Lactose monohydrate | 25.96 | N/A | N/A | N/A | N/A |
| Microcrystalline cellulose | N/A | 23.4025 | 17.94 | 25.4375 | 19.5 |
| Mannitol | N/A | 8.05 | 20.9875 | 8.75 | 22.8125 |
| Dibasic calcium phosphate, anhydrous | N/A | 7.475 | N/A | 8.125 | N/A |
| Sodium starch glycolate | 1.92 | N/A | N/A | N/A | N/A |
| Low-substituted hydroxpropylcellulose | N/A | 2.875 | 2.875 | 3.125 | 3.125 |
| Magnesium Stearate | 0.57 | 0.8625 | 0.8625 | N/A | N/A |
| Glyceryl Dibehenate | N/A | N/A | N/A | 0.9375 | 0.9375 |

^{a} Micronised acalabrutinib maleate
^{b} Acalabrutinib maleate particle size, D_{(v,} _{0.9)} ≈ 70 µm

TABLE 4

COMPOSITION OF DIRECT COMPRESSION TABLETS

| Component | T6 (g/batch) EB18-432407 | T7 (g/batch) EB19-021246 | T8 (g/batch) EB18-431421 | T9 (g/batch) EB19-033106 | T10 (g/batch) EB19-033107 | T11 (g/batch) EB19-033101 | T12 (g/batch) EB19-033108 | T13 (g/batch) EB19-033109 | T14 (g/batch) EB19-033110 | T15 (g/batch) EB19-021245 |
|---|---|---|---|---|---|---|---|---|---|---|
| Acalabrutinib maleate | 16.125^{a} | 12.9^{a} | 16.125^{a} | 21.5^{a} | 15.48^{b} | 7.74^{c} | 21.5^{b} | 15.48^{d} | 21.5^{d} | 12.9^{a} |
| Microcrystalline cellulose | 24.5 | 19.6 | 18.563 | 11 | 17.82 | 8.91 | 11 | 17.82 | 11 | 14.9 |
| Mannitol | 8.75 | 7 | 22.8125 | 13.5 | 21.9 | 10.95 | 13.5 | 21.9 | 13.5 | 18.3 |
| DPCA | 8.125 | 6.5 | N/A | N/A | N/A | N/A | N/A | N/A | N/A | N/A |
| Low-substituted hydroxpropyl cellulose | 3.125 | 2.5 | 3.125 | 2.5 | 3 | 1.5 | 2.5 | 3 | 2.5 | 2.5 |
| Sodium stearyl fumarate | 1.875 | 1.5 | 1.875 | 1.5 | 1.8 | 0.9 | 1.5 | 1.8 | 1.5 | 1.5 |

^{a} Acalabrutinib maleate particle size, D_{(v,} _{0.9)} ≈ 70 µm
^{b} Acalabrutinib maleate particle size, D_{(v,} _{0.9)} ≈ 150 µm
^{c} Acalabrutinib maleate particle size, D_{(v,} _{0.9)} ≈ 16 µm
^{d} Acalabrutinib maleate particle size, D_{(v,} _{0.9)} ≈ 500 µm

### B. Roller Compaction Tablets

Tablets having the compositions set forth in Table 5 were prepared by roller compaction. All components, except for the lubricant, were blended. The intra-granular portion of lubricant was screened and then added to the blend, which was then lubricated by further blending. The lubricated blend was roller compacted to form ribbons, which were subsequently milled to granules. The extra-granular portion of lubricant was screened and then added to the granules, which were then lubricated by further blending. Tablet cores were compressed to a target compression weight and force of 400 mg and 14 kN using 13 x 7.5 mm oval tablet tooling. The resultant tablet cores were film coated with a 3% to 4% weight gain of the coating suspension.

TABLE 5

COMPOSITION OF FILM-COATED ROLLER COMPACTION TABLETS

| Component | T16 (kg/batch) TAAB | T17 (kg/batch) TAAC | T18 (kg/batch) TAAD | T19 (kg/batch) EB19-144101 | T20 (g/batch) EB19-079701 |
|---|---|---|---|---|---|
| Acalabrutinib maleate | 12.9^{a} | 12.9^{b} | 12.9^{c} | 1000^{d} | 241.9^{e} |
| Microcrystalline cellulose | 10.9 | 10.9 | 10.9 | 845 | 202.5 |
| Mannitol | 13.2 | 13.2 | 13.2 | 1023.3 | 245.6 |
| Low-substituted hydroxpropyl cellulose | 2 | 2 | 2 | 155 | 37.5 |
| Sodium stearyl fumarate (intra-granular) | 0.2 | 0.2 | 0.2 | 15.5 | 3.8 |
| Sodium stearyl fumarate (extra-granular) | 0.8 | 0.8 | 0.8 | 58 | 18.8 |
| Film coat (% weight gain) | 3.4 | 3.7 | 3.7 | 4.0 | N/A |

^{a} Acalabrutinib maleate particle size, D_{(v,} _{0.9)} ≈ 260 µm
^{b} Acalabrutinib maleate particle size, D_{(v,} _{0.9)} ≈ 198 µm
^{c} Acalabrutinib maleate particle size, D_{(v,} _{0.9)} ≈ 270 µm
^{d} Acalabrutinib maleate particle size, 50:50 mix of D_{(v,} _{0.9)} ≈ 70 µm and D_{(v, 0.9)} ≈ 150 µm
^{e} Acalabrutinib maleate particle size, D_{(v,} _{0.9)} ≈ 70 µm

### C. Roller Compacted Capsules

In addition to the tablets described above, reference capsules comprising acalabrutinib free base and having the compositions set forth in Table 6 were prepared and employed in several of the following Examples. All components, except for the lubricant, were blended, then screened through a sieve, and then blended again. Screened lubricant was added to the blend, which was then lubricated by further blending. The lubricated blend was fed into a roller compactor and the resultant ribbon was subsequently milled to produce granules suitable for encapsulation. Screened extra-granular lubricant was blended with the acalabrutinib granules which, once lubricated, were filled using an encapsulator into size 1 hard gelatin capsules to a target fill weight of 240 mg (i.e., 100 mg acalabrutinib free base).

TABLE 6

COMPOSITION OF ROLLER COMPACTED CAPSULES

| Component | C1 (kg/batch) | C2 (kg/batch) | C3 (kg/batch) | C4 (kg/batch) |
|---|---|---|---|---|
| | W025985 | W026394 | WO27180 | L0505009 |
| Acalabrutinib | 10.951^{a} | 10.951^{b} | 11.130^{c} | 11.765^{d} |
| Silicified Microcrystalline Cellulose | 7.227 | 7.227 | 7.324 | 7.655 |
| Partially Pregelatinised Starch | 7.227 | 7.227 | 7.324 | 7.655 |
| Sodium Starch Glycollate | 0.657 | 0.657 | 0.666 | 0.696 |
| Magnesium Stearate (intra-granular) | 0.109 | 0.109 | 0.111 | 0.116 |
| Magnesium Stearate (extra-granular) | 0.109 | 0.109 | 0.111 | 0.114 |
| Total capsule fill weight into Size 1 blue/yellow gelatin capsules | 240 mg | 240 mg | 240 mg | 240 mg |

^{a} Acalabrutinib free base particle size, D_{(v,} _{0.9)} ≈ 365 µm
^{b} Acalabrutinib free base particle size, D_{(v,} _{0.9)} ≈ 392 µm
^{c} Acalabrutinib free base particle size, D_{(v,} _{0.9)} ≈ 394 µm
^{d} Acalabrutinib free base particle size, D_{(v,} _{0.9)} ≈ 377 µm

### D. Film-Coated Tablet T21

A further example of a film-coated dosage form (T21) is described in Table 7 below.

TABLE 7

ACALABRUTINIB MALEATE FILM-COATED TABLETS (T21)

| COMPONENTS | % W/W | QUANTITY (MG/TABLET) | FUNCTION | GRADE |
|---|---|---|---|---|
| TABLET CORE | | | | |
| Acalabrutinib maleate monohydrate Form A | 32.25 | 129.0* | Active | |
| Mannitol | 33.00 | 132.0 | Filler | Pearlitol 200SD |
| Microcrystalline cellulose | 27.25 | 109.0 | Filler | Avicel PH102 |
| Low-substituted hydroxpropyl cellulose | 5.00 | 20.0 | Disintegrant | LH-31 |
| Sodium stearyl fumarate | 0.50 | 2.0 | Intra-Granular Lubricant | PRUV SSF |
| Sodium stearyl fumarate | 2.00 | 8.0 | Extra-Granular Lubricant | |
| Core Tablet Weight | | 400.0 | | |
| TABLET COATING | | | | |
| Aquarius Preferred BPP313095 | 3.5 | 14.0 | Film coat | Ashland |
| Nominal Coated Tablet Weight | | 414.0 | | |

* 100 mg free base equivalent weight.

### Example 5: Assessment of In Vitro Dissolution Profile

In vitro dissolution studies were conducted to assess the dissolution profiles of acalabrutinib maleate formulations under low pH conditions and under elevated pH conditions. The pH conditions were selected to simulate the gastric pH conditions where the tablet is administered alone (low pH conditions) or co-dosed with a proton pump inhibitor or acid reducing agent (elevated pH conditions). Details of the dissolution studies are provided below.

### 1. Low pH 0.1 N HCL Dissolution Test

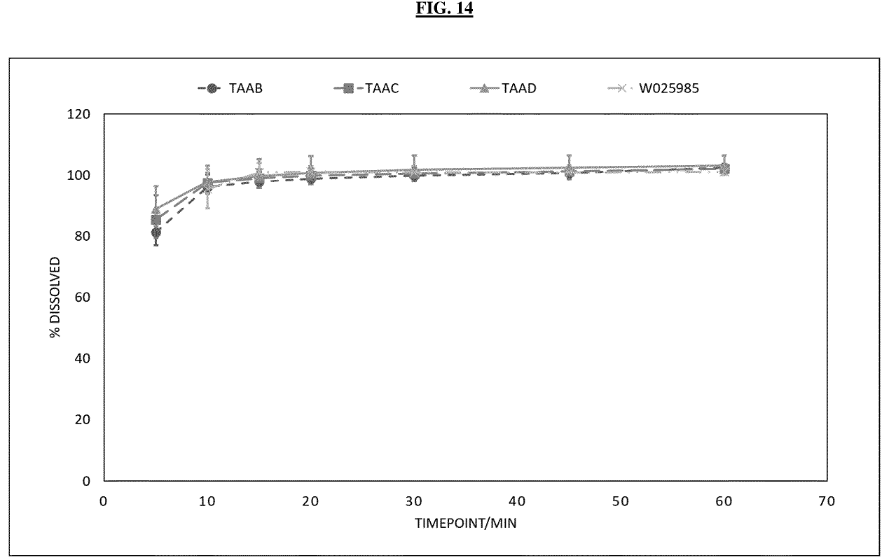

FIG. 14 shows dissolution profiles obtained from a low pH test under sink conditions for acalabrutinib maleate tablets T16, T17, and T18, and acalabrutinib free base capsule C1. The dissolution testing was conducted in 900 mL of dissolution medium containing 0.1 N hydrochloric acid and using USP dissolution apparatus 2 (paddle) operating at 50 RPM at 37 ± 0.5° C. Samples from the dissolution medium were pulled from the aqueous phase at predetermined time points and assayed by either HPLC or UV/visible spectroscopy. The results show that under low pH conditions the acalabrutinib maleate tablets and acalabrutinib free base capsule have similar dissolution profiles.

### 2. Neutral pH Low Ionic Strength 5 mM Phosphate pH 6.8 Dissolution Test

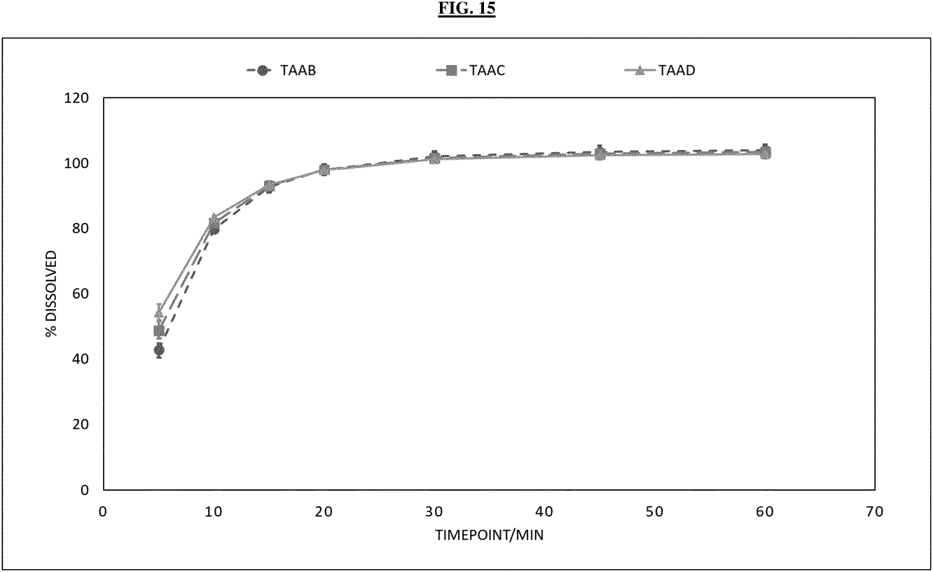

FIG. 15 shows dissolution profiles obtained from a neutral pH low ionic strength test under sink conditions for acalabrutinib maleate tablets T16, T17, and T18. The dissolution testing was conducted in 900 mL of dissolution medium containing 5 mM sodium phosphate adjusted to pH 6.8 and using USP dissolution apparatus 2 (paddle) operating at 75 RPM at 37 ± 0.5° C. Samples from the dissolution medium were pulled from the aqueous phase at predetermined time points and assayed by UV/visible spectroscopy. The results show that these acalabrutinib maleate tablets substantially retained the dissolution profile exhibited under the low pH conditions when they were tested under the elevated pH conditions.

### 3. Neutral pH High Ionic Strength 50 mM Phosphate pH 6.8 Dissolution Test

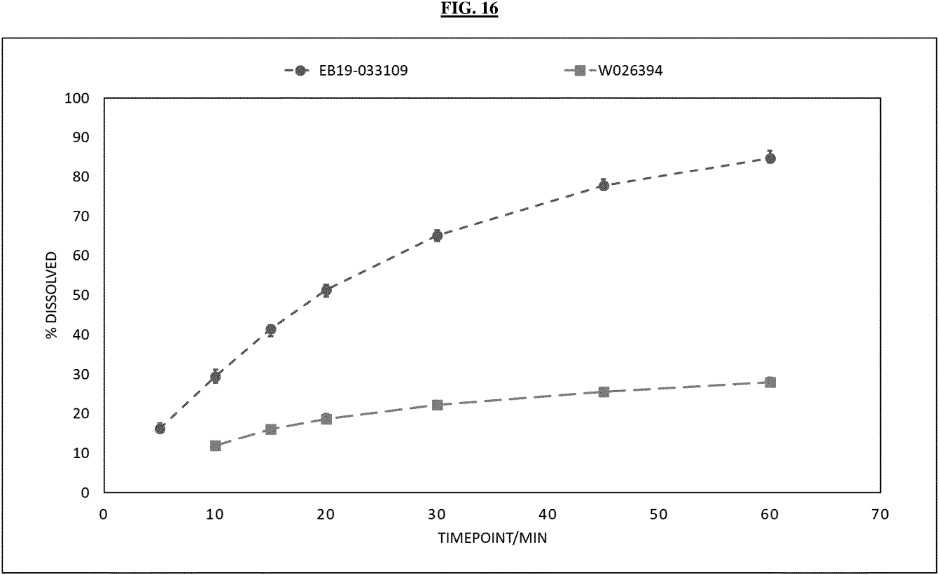

FIG. 16 shows dissolution profiles obtained from a neutral pH high ionic strength test for acalabrutinib maleate tablet T13 and acalabrutinib free base capsule C2. The dissolution testing was conducted in 900 mL of dissolution medium containing 50 mM sodium phosphate adjusted to pH 6.8 and using USP dissolution apparatus 2 (paddle) operating at 75 RPM at 37 ± 0.5° C. Samples from the dissolution medium were pulled from the aqueous phase at predetermined time points and assayed by HPLC. The results show an improved dissolution profile under elevated pH conditions for the acalabrutinib maleate tablet relative to the acalabrutinib free base capsule.

### 4. Water Dissolution Test

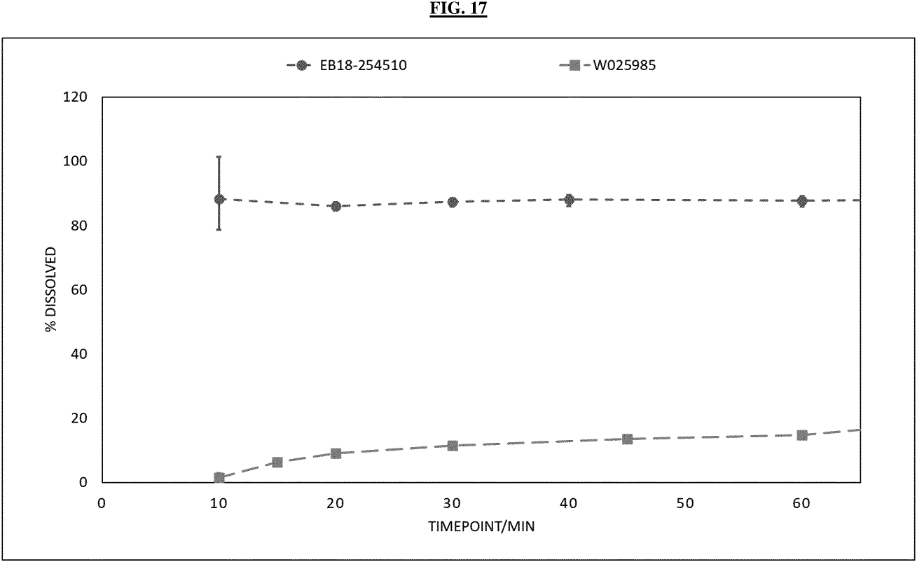

FIG. 17 shows the dissolution profiles obtained from a neutral medium with no buffer capacity (i.e., conditions similar to a proton pump inhibitor-treated stomach) for acalabrutinib maleate tablet T1 and acalabrutinib free base capsule C1. The dissolution testing was conducted in 300 mL of dissolution medium containing deionized water and using USP dissolution apparatus 2 (paddle) operating at 50 RPM and 37 ± 0.5° C. Samples from the dissolution medium were pulled from the aqueous phase at predetermined time points and assayed by HPLC.

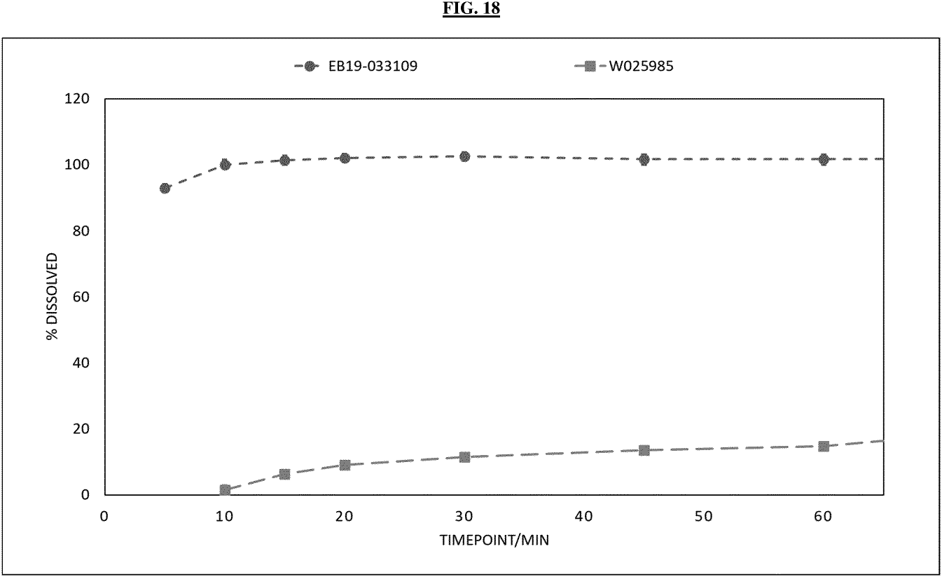

FIG. 18 shows the dissolution profiles obtained from a neutral medium with no buffer capacity for acalabrutinib maleate tablet T13 and acalabrutinib free base capsule C1. The dissolution testing for tablet T13 was conducted in 900 mL of dissolution medium volume containing deionized water and using USP dissolution apparatus 2 (paddle) operating at 75 RPM and 37 ± 0.5° C. and compared to reference tablet C1 for which the testing was conducted in 300 mL and 50 RPM. Samples from the dissolution medium were pulled from the aqueous phase at predetermined time points and assayed by HPLC.

The results presented in FIGS. 17 and 18 show an improved dissolution profile under elevated pH conditions for the acalabrutinib maleate tablets relative to the acalabrutinib free base capsule.

### 5. Biorelevant Medium Test

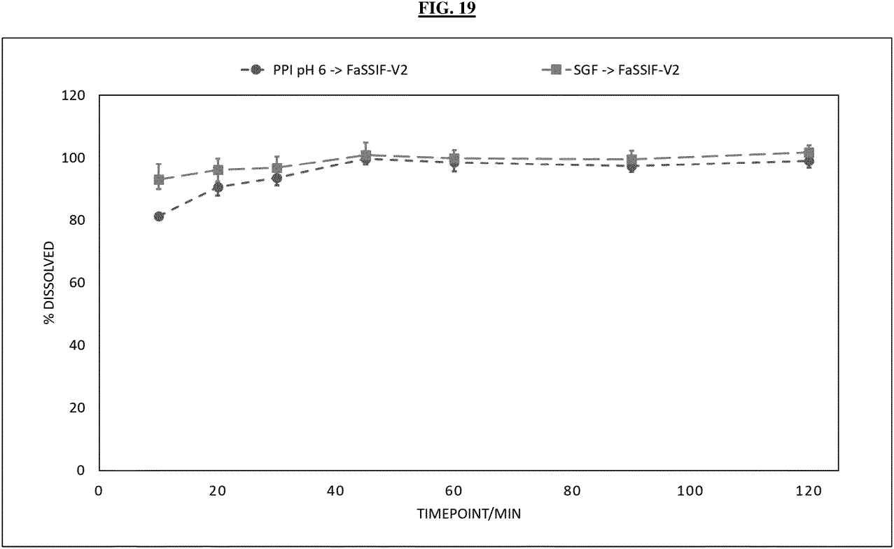

Dissolution of acalabrutinib maleate tablet T19 was evaluated under gastric conditions associated with an acidic gastric compartment and also under gastric conditions associated with dosing in combination with a proton pump inhibitor or acid reducing agent. The initial medium employed was either a simulated gastric fluid containing hydrochloric acid and sodium chloride and pH adjusted to 1.8 or a low buffer capacity medium designed to replicate a proton pump inhibitor treated stomach (see Segregur D., et al., “Impact of Acid-Reducing Agents on Gastrointestinal Physiology and Design of Biorelevant Dissolution Tests to Reflect These Changes,” J. Pharm. Sci., 108(11): 2461-3477 (2019)). The PPI buffer was maleate based and contained sodium chloride adjusted to pH 6. After tablet T19 had been present in the initial medium for 30 minutes, the medium was converted to a FaSSIF-V2 medium by addition of a double strength concentrate to give a final pH of 6.5. The FaSSIF-V2 medium contained a sodium phosphate buffer with sodium chloride, sodium taurocholate, and lecithin. The dissolution testing was conducted using USP dissolution apparatus 2 (paddle) operating at 75 RPM at 37 ± 0.5° C. in 250 mL for first 30 minutes and then 500 mL post shift. Samples from the dissolution medium were pulled from the aqueous phase at predetermined time points and assayed by HPLC. Following the pH shift to FaSSIF-V2 for both of the starting media the acalabrutinib (at 100 mg free base equivalent dose) did not precipitate and supersaturated for at least a further 90 minutes as evidenced by FIG. 19.

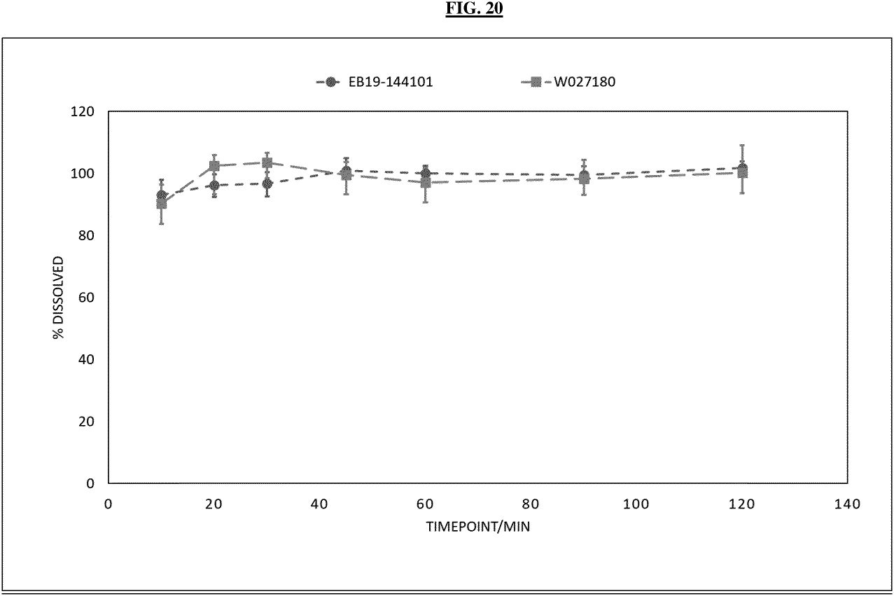

In a separate dissolution test, acalabrutinib maleate tablet T19 and acalabrutinib free base capsule C3 were evaluated under the same pH shift conditions as described above using simulated gastric fluid pH 1.8 as the initial medium. FIG. 20 reports the results which indicate that the maleate tablet has an in vitro dissolution performance under biorelevant conditions corresponding to a fasted-state stomach that is comparable to the free base capsule.

Overall, the results from the in vitro dissolution tests indicate that the dissolution profiles of the acalabrutinib maleate tablets tested under low pH conditions and elevated pH conditions are substantially comparable, further suggesting that such tablets when administered alone or when co-administered with a proton pump inhibitor or acid reducing agent are bioequivalent.

### Example 6: Assessment In TIM-1 Model

A study was conducted using the TNO TIM-1 (TIM-1) system, an important tool in the testing cascade for building a mechanistic understanding of in vitro product performance and demonstrating the clinical relevance of the selected in vitro method. The TIM-1 system has been previously described in detail in the literature. See, e.g., Barker, R., et al., “Application and validation of an advanced gastrointestinal in vitro model for the evaluation of drug product performance in pharmaceutical development,” J. Pharm. Sci., Volume 103, Issue 11, 15, Pages 3704-3712 (September 2014). The TIM-1 system is a multicompartmental, dynamic system that makes use of in-vivo relevant media, volumes, pH and hydrodynamics so as to mimic the conditions found in the upper GI-tract of an adult human. The system also mimics absorptive sink by means of hollow fibre ultrafiltration. Volumes, media composition, emptying rates, temperature and pH are all dynamically computer controlled, allowing the definition of various subject physiologies, such as fasted, fed or other various more complex disease states.

More specifically, the present study was conducted in the TIM-1 system to assess the relative performance of acalabrutinib maleate tablet T19 and acalabrutinib free base capsule C2, evaluated under gastric conditions associated with an acidic gastric compartment and also under gastric conditions associated with dosing in combination with a proton pump inhibitor or acid reducing agent. The selected conditions represented a human with a gastric pH of 2 and 6. The gastric emptying rate was set in the “rapid” mode, to represent the most challenging scenario for the formulations from a pH shift perspective. This means the stomach compartment t_{½} was 15 minutes which is typical of the in vivo scenario for a fasted adult. The TIM-1 system was dosed with the test article and the selected protocol was run for 300 minutes. The system then ran automatically, and samples from the absorptive compartments were collected and assayed every 60 minutes by HPLC.

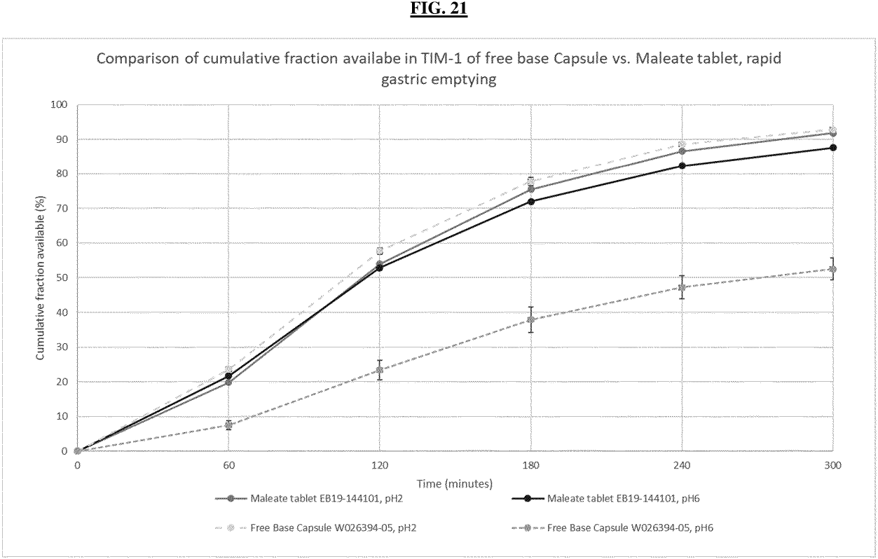

FIG. 21 demonstrates that the acalabrutinib maleate tablet performance was equivalent to that of the acalabrutinib free base capsule in the low pH (pH 2) condition. It also demonstrates the acalabrutinib maleate tablet performance was unaffected by the high pH (pH 6) condition and did not precipitate upon the pH shift that occurred with gastric emptying into the duodenum.

### Example 7: Impact of Particle Size and Drug Load on Dissolution Rate

A study was conducted to evaluate the impact of drug substance particle size and drug substance loading on in vitro dissolution of acalabrutinib maleate tablets. The tablets evaluated contained acalabrutinib maleate (100 mg free base equivalent) with a D_{(v,} _{0.9)} particle size (measured by laser diffraction) ranging from 16 microns to 500 microns and a drug loading of either 26 weight% or 43 weight%. The dissolution testing was conducted in 900 mL of 5 mM sodium phosphate buffer medium using USP2 dissolution apparatus (paddle) at operating at 75 RPM and 37 ± 0.5° C.

Acalabrutinib maleate tablets T9, T10, T11, T12, T13, T14, and T15 were evaluated in the study. The drug substance particle size and drug loading for each tablet are summarized in Table 8 below.

TABLE 8

| Tablet | T9 | T10 | T11 | T12 | T13 | T14 | T15 |
|---|---|---|---|---|---|---|---|
| | EB19-033106 | EB19-033107 | EB19-033101 | EB19-033108 | EB19-033109 | EB19-033110 | EB19-021245 |
| D_{(v,} _{0.9)} Particle Size (µm) | 70 | 150 | 16 | 150 | 500 | 500 | 70 |
| Drug Loading (Weight %) | 43 | 26 | 26 | 43 | 26 | 43 | 26 |

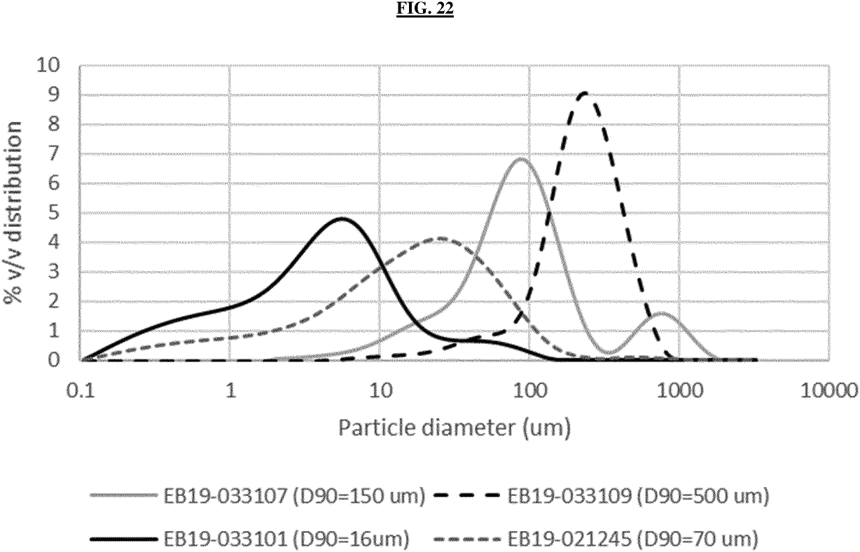

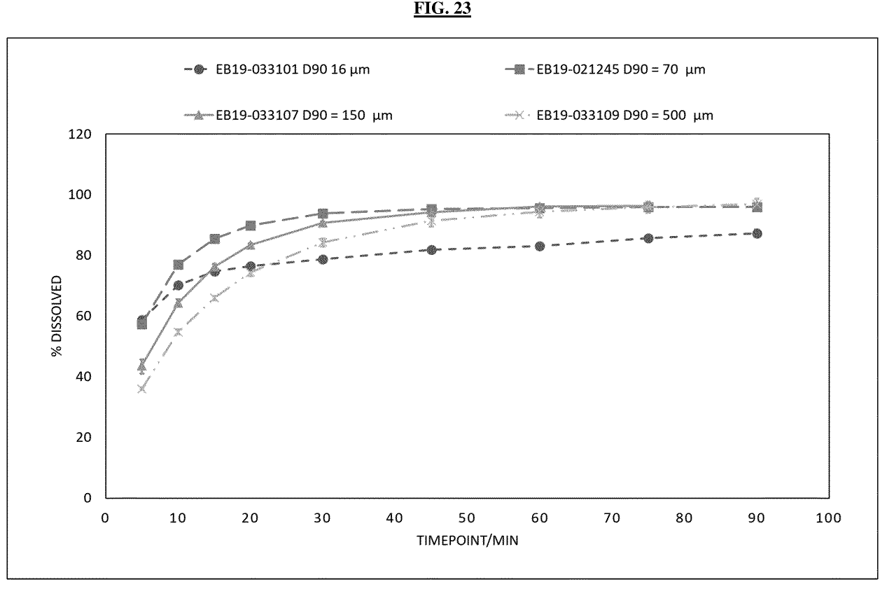

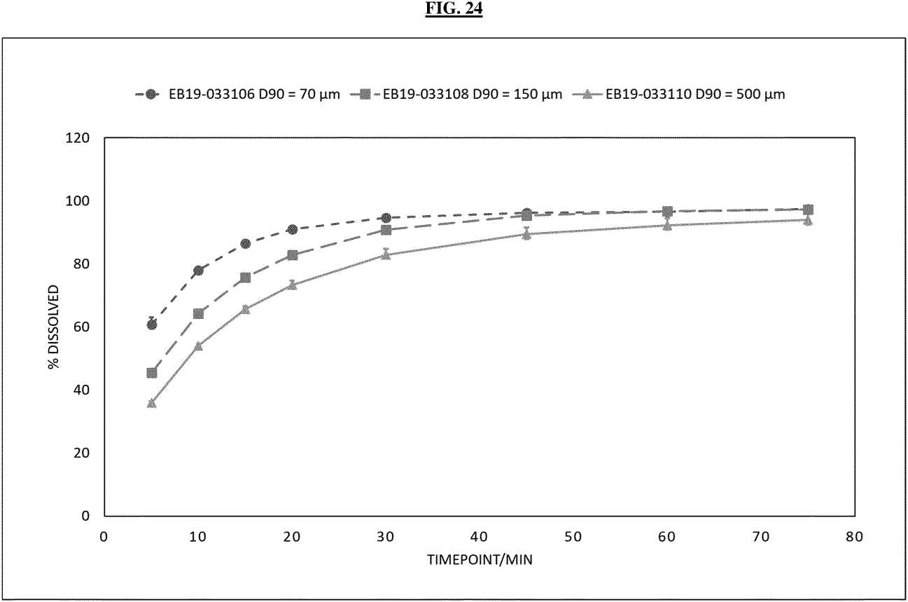

FIG. 22 additionally shows the particle size distributions for acalabrutinib maleate tablets T10, T11, T13, and T15. The tablets evaluated for impact of drug loading were acalabrutinib maleate tablets T10, T11, T13, and T15 (drug loading of 26 weight%) and acalabrutinib maleate tablets T9, T2, and T14 (drug loading of 43% weight %), respectively. FIGS. 23 and 24 show the results of the dissolution testing for acalabrutinib maleate tablets T10, T11, T13, and T15 (drug loading of 26 weight%) and acalabrutinib maleate tablets T9, T12, and T14 (drug loading of 43% weight %), respectively. The tablet dissolution rate generally decreased as acalabrutinib maleate particle size increased although this observation did not hold true for the tablet with the finest acalabrutinib particle size (T11). One possible explanation for the difference in the tablet T11 result is that the dissolution rate, which was rapid at the initial time points, was reduced due to a lack of drug wettability. The in vitro dissolution rates for acalabrutinib maleate tablets with particle size distributions in the range of D_{(v,} _{0.9)} of 70 µm to 500 µm under the conditions tested were relatively consistent when drug loading increased from 26 weight% to 43 weight%.

### Example 8: GastroPlus Modeling and Simulation of Acalabrutinib Exposure

A software modeling and simulation study was conducted to predict acalabrutinib exposure in a human subject after administration of the acalabrutinib maleate tablets of Example 7 (i.e., T10, T11, T13, and T15). The tablet dissolution rate data obtained in Example 7 were used to derive a batch specific drug product particle size distribution (“P-PSD”) for each tablet according to the methodology described by Pepin, et al. (Pepin, X.J.H., et al., “Bridging in vitro dissolution and in vivo exposure for acalabrutinib. Part I. Mechanistic modelling of drug product dissolution to derive a P-PSD for PBPK model input,” Eur. J. Pharm. Biopharm., 142:421-434 (2019)) and a measured solubility of 2.144 mg/mL. The derived P-PSD were then used as an input to a PBPK model described by Pepin et al. (Pepin, X. J. H., et al. “Bridging in vitro dissolution and in vivo exposure for acalabrutinib. Part II. A mechanistic PBPK model for IR formulation comparison, proton pump inhibitor drug interactions, and administration with acidic juices,” Eur. J. Pharm. and Biopharm., 142: 435-448 (2019)) to predict the human exposure to acalabrutinib for each of the tablets.

The simulation predicted that the T10, T11, T13, and T15 tablets under acidic stomach conditions at 100 mg free base equivalent all had average AUC and Cₘₐₓ values that were comparable to the average AUC and Cₘₐₓ values for the acalabrutinib free base reference capsule C4. Table 9 below summarizes the calculated average exposure values for the acalabrutinib maleate tablets and the ratios of those calculated values to the corresponding values of the acalabrutinib free base reference capsule. The exposure ratio for the T11 tablet was close to the lower limit for bioequivalence, possibly due to a slower dissolution rate related to wettability issues.

TABLE 9

| Condition | Batch | Parameter | Average | SD | Ratio to ref | |
|---|---|---|---|---|---|---|
| Acidic | L0505009 | Pred AUC(0-t) (ng-h/mL) | 700 | 215 | 1 | Free base |
| Acidic | EB19-021245 | Pred AUC(0-t) (ng-h/mL) | 679 | 201 | 0.97 | D90=70 um |
| Acidic | EB19-033101 | Pred AUC(0-t) (ng-h/mL) | 630 | 171 | 0.9 | D90=16 um |
| Acidic | EB19-033107 | Pred AUC(0-t) (ng-h/mL) | 675 | 198 | 0.96 | D90=150 um |
| Acidic | EB19-033109 | Pred AUC(0-t) (ng-h/mL) | 662 | 189 | 0.95 | D90=500 um |
| Acidic | L0505009 | Pred C_{Max} (ng/mL) | 649 | 299 | 1 | Free base |
| Acidic | EB 19-021245 | Pred C_{Max} (ng/mL) | 614 | 276 | 0.95 | D90=70 um |
| Acidic | EB19-033101 | Pred C_{Max} (ng/mL) | 534 | 229 | 0.82 | D90=16 um |
| Acidic | EB19-033107 | Pred C_{Max} (ng/mL) | 607 | 270 | 0.93 | D90=150 um |
| Acidic | EB19-033109 | Pred C_{Max} (ng/mL) | 586 | 256 | 0.9 | D90=500 um |

A similar simulation predicted that the T10, T11, T13, and T15 tablets under neutral to acidic stomach conditions at 100 mg free base equivalent all had average AUC and Cₘₐₓ values that were substantially retained over the pH range relative to the average AUC and Cₘₐₓ values for the acalabrutinib free base reference capsule C4 over the same pH range. The simulation supported the conclusion that that the effect of acid reducing agents on acalabrutinib exposure can be substantially reduced relative to the acalabrutinib free base reference capsule C4 with the acalabrutinib maleate tablets maintaining bioequivalence over the acidic to neutral pH range. Table 10 below summarizes the calculated average exposure values for the acalabrutinib maleate tablets and the ratios of those calculated values to the corresponding values of the acalabrutinib free base reference capsule.

TABLE 10

| D90 (um) | Condition | Parameter | Average | SD | Ratio to acidic |
|---|---|---|---|---|---|
| 70 | EB19-021245 - Acidic | Pred AUC(0-t) (ng-h/mL) | 679 | 201 | |
| 70 | EB 19-021245 - Neutral | Pred AUC(0-t) (ng-h/mL) | 655 | 184 | 0.96 |
| 16 | EB19-033101 - Acidic | Pred AUC(0-t) (ng-h/mL) | 630 | 171 | |
| 16 | EB19-033101 - Neutral | Pred AUC(0-t) (ng-h/mL) | 594 | 151 | 0.94 |
| 150 | EB19-033107 - Acidic | Pred AUC(0-t) (ng-h/mL) | 675 | 198 | |
| 150 | EB19-033107 - Neutral | Pred AUC(0-t) (ng-h/mL) | 637 | 172 | 0.94 |
| 500 | EB19-033109 - Acidic | Pred AUC(0-t) (ng-h/mL) | 662 | 189 | |
| 500 | EB19-033109 - Neutral | Pred AUC(0-t) (ng-h/mL) | 611 | 156 | 0.92 |
| Free base | L0505009 - Acidic | Pred AUC(0-t) (ng-h/mL) | 700 | 215 | |
| Free base | L0505009 - Neutral | Pred AUC(0-t) (ng-h/mL) | 259 | 58 | 0.37 |
| 70 | EB 19-021245 - Acidic | Pred C_{Max} (ng/mL) | 614 | 276 | |
| 70 | EB 19-021245 - Neutral | Pred C_{Max} (ng/mL) | 571 | 244 | 0.93 |
| 16 | EB19-033101 - Acidic | Pred C_{Max} (ng/mL) | 534 | 229 | |
| 16 | EB19-033101 - Neutral | Pred C_{Max} (ng/mL) | 473 | 194 | 0.89 |
| 150 | EB19-033107 - Acidic | Pred C_{Max} (ng/mL) | 607 | 270 | |
| 150 | EB19-033107 - Neutral | Pred C_{Max} (ng/mL) | 539 | 220 | 0.89 |
| 500 | EB19-033109 - Acidic | Pred C_{Max} (ng/mL) | 586 | 256 | |
| 500 | EB19-033109 - Neutral | Pred C_{Max} (ng/mL) | 495 | 193 | 0.85 |
| Free base | L0505009 - Acidic | Pred C_{Max} (ng/mL) | 649 | 299 | |
| Free base | L0505009 - Neutral | Pred C_{Max} (ng/mL) | 138 | 38 | 0.21 |

### Example 9: In Vivo Dog Study

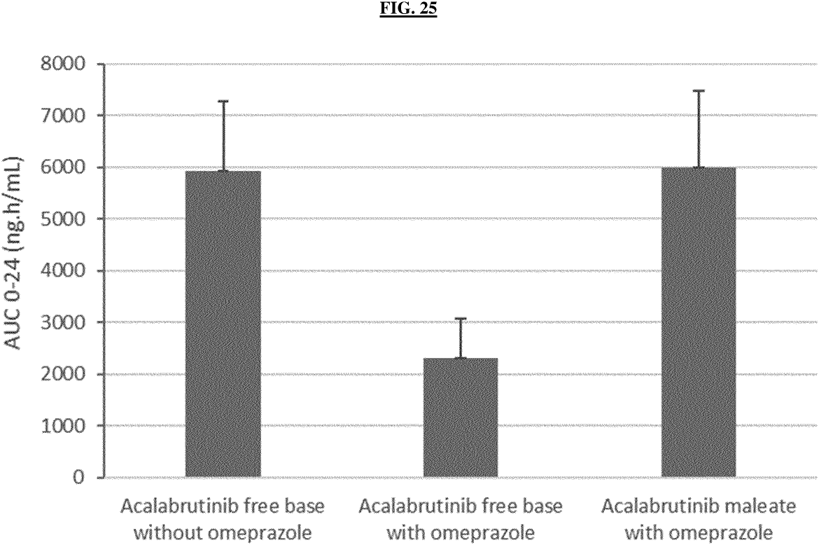

An in vivo study was conducted to evaluate coadministration of acalabrutinib maleate and omeprazole relative to coadministration of acalabrutinib free base and omeprazole in a dog model. In the study, non-naive beagle dogs were dosed with capsules containing 100 mg of acalabrutinib free base, both with and without 10 mg omeprazole pre-treatment, and acalabrutinib AUC₍₀₋₂₄₎ values measured. In addition, the same dogs after an appropriate wash-out period were dosed with Size 13 capsules containing a binary mixture of acalabrutinib maleate (100 mg equivalent weight) and 200 mg microcrystalline cellulose, both with and without omeprazole pre-treatment, and acalabrutinib AUC₍₀₋₂₄₎ values measured. The study results are depicted in FIG. 25. The acalabrutinib maleate capsules (100 mg free base equivalent weight) when dosed with omeprazole pre-treatment maintained comparable exposure to the acalabrutinib free base capsules when dosed without omeprazole pre-treatment.

### Example 10: Assessment of Excipients and Excipient Combinations

A study was conducted to evaluate the suitability of certain excipients and excipient combinations in formulating an acalabrutinib maleate dosage form.

### A. Disintegrants

Binary mixes of disintegrants and acalabrutinib maleate (1:5 ratio) were prepared and evaluated in in vitro dissolution testing. The binary blends and an acalabrutinib maleate control were dissolved in 250 mL of deionized water using USP2 dissolution apparatus (paddle) at 37 ± 0.5° C. and 75 RPM. After the 120-minute timepoint, the paddle speed was increased to 250 RPM and after 135 minutes the pH was adjusted to pH 1.8-2 to increase solubility in order to determine whether any undissolved material remained. The binary mixes tested were sodium starch glycolate/ acalabrutinib maleate (1:5 ratio), croscarmellose sodium/acalabrutinib maleate (1:5 ratio), and low-substituted hydroxypropyl cellulose/acalabrutinib maleate (1:5 ratio).

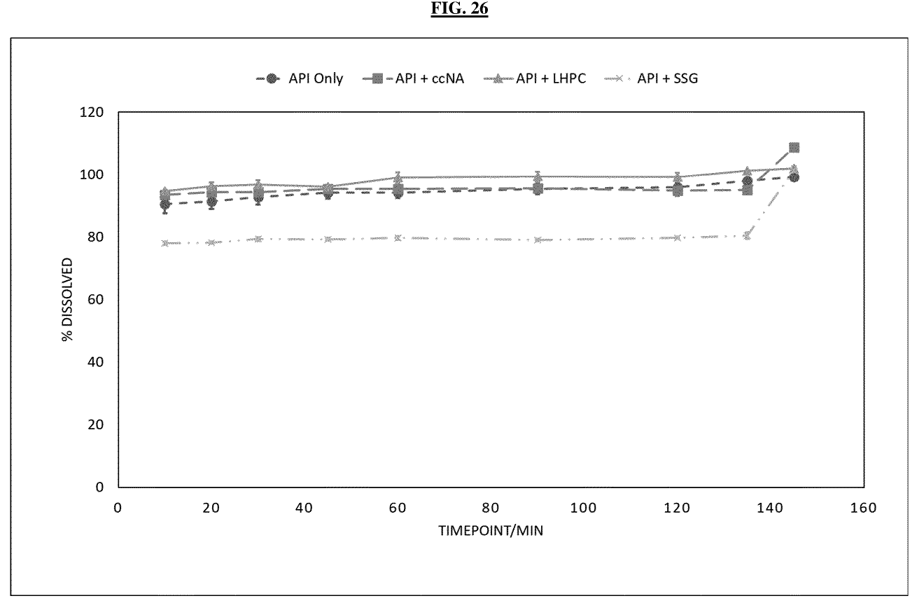

Results are shown in FIG. 26. Only the acalabrutinib maleate control and the low-substituted hydroxypropyl cellulose/acalabrutinib maleate (1:5 ratio) mix exhibited no significant increase in dissolution after the increase in paddle speed or addition of acid suggesting that completion dissolution had been achieved. For the croscarmellose sodium/acalabrutinib maleate (1:5 ratio) mix and the croscarmellose sodium/acalabrutinib maleate (1:5 ratio) mix, a significant increase in dissolution was exhibited upon acid adjustment and demonstrated that full release could be a problem at higher pH levels and suggested that an excipient/drug substance interaction, possibly caused by conversion of acalabrutinib maleate to a less soluble form such as the free base, was taking place.

### B. Lubricants

Binary mixes of lubricants and acalabrutinib maleate (1:15) were prepared and evaluated in in vitro dissolution testing under the same conditions described above for the disintegrant mixes. The binary mixes tested were glyceryl dibehenate/acalabrutinib maleate (1:15), magnesium stearate/acalabrutinib maleate (1:15), and sodium stearyl fumarate/acalabrutinib maleate (1:15).

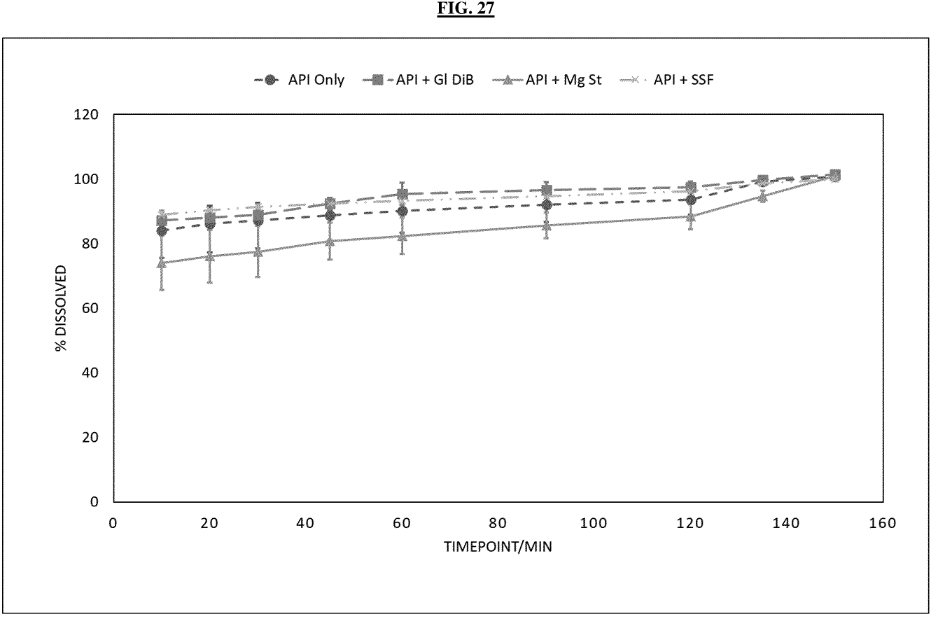

Results are shown in FIG. 27. The acalabrutinib maleate control, glyceryl dibehenate/acalabrutinib maleate (1:15) mix, and sodium stearyl fumarate/acalabrutinib maleate (1:15) mix exhibited no significant increase in dissolution after the increase in paddle speed or addition of acid suggesting that completion dissolution had been achieved. For the magnesium stearate/acalabrutinib maleate (1:15) mix, a significant increase in dissolution was observed upon increase in paddle speed and acid adjustment demonstrated that full release could be a problem at higher pH levels and suggested that an excipient/drug substance interaction, possibly caused by conversion of acalabrutinib maleate to a less soluble form such as the free base, was taking place. Additionally, when binary compacts of magnesium stearate and acalabrutinib maleate were evaluated, those binary compacts showed an increase in the extent of acalabrutinib degradation compared to acalabrutinib maleate alone.

### C. Diluent

Direct compression tablet cores containing diluent, disintegrant, lubricant, and acalabrutinib maleate were prepared and evaluated in in vitro dissolution testing under the same conditions described above for the disintegrant mixes. Each tablet core contained either microcrystalline cellulose/mannitol or microcrystalline cellulose/dibasic calcium phosphate anhydrous/mannitol as the diluent. The specific tablet core tested contained (1) microcrystalline cellulose, dibasic calcium phosphate anhydrous, mannitol, low-substituted hydroxypropyl cellulose, magnesium stearate, and acalabrutinib maleate (T2), (2) microcrystalline cellulose, mannitol, low-substituted hydroxypropyl cellulose, magnesium stearate, and acalabrutinib maleate (T3), (3) microcrystalline cellulose, dibasic calcium phosphate anhydrous, mannitol, low-substituted hydroxypropyl cellulose, sodium stearyl fumarate, and acalabrutinib maleate (T6), (4) microcrystalline cellulose, mannitol, low-substituted hydroxypropyl cellulose, sodium stearyl fumarate, and acalabrutinib maleate (T8), (5) microcrystalline cellulose, dibasic calcium phosphate anhydrous, mannitol, low-substituted hydroxypropyl cellulose, glyceryl dibehenate, and acalabrutinib maleate (T4), or (6) microcrystalline cellulose, mannitol, low-substituted hydroxypropyl cellulose, glyceryl dibehenate, and acalabrutinib maleate (T5).

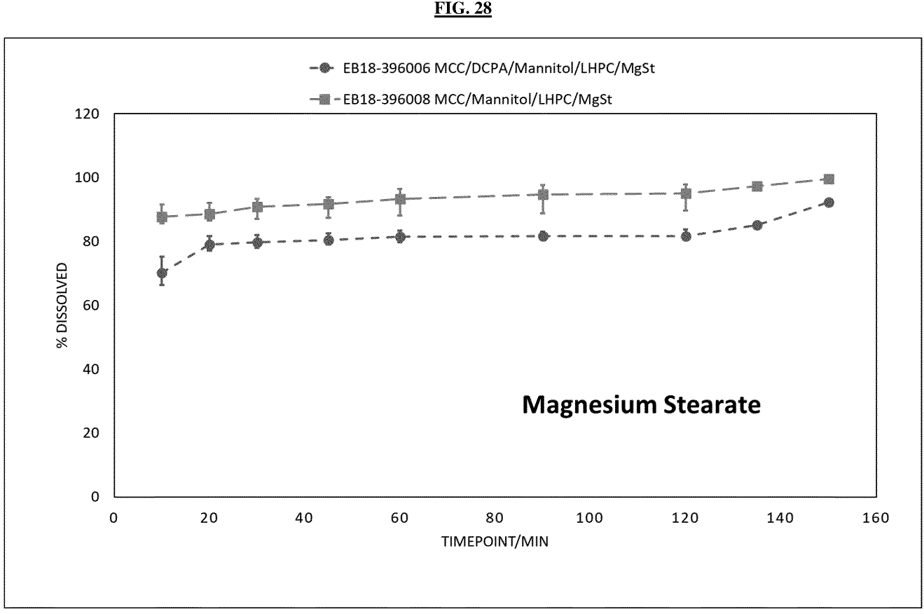

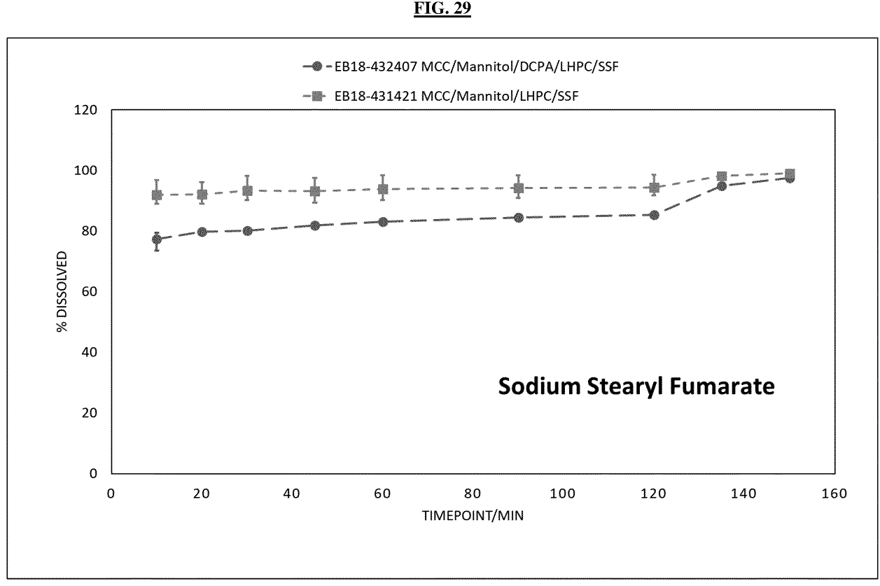

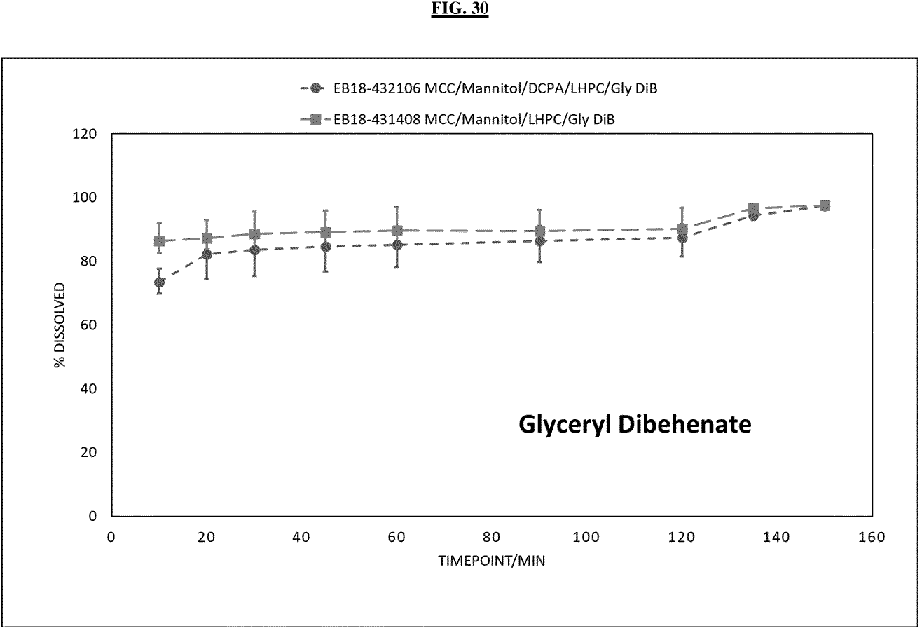

Results are shown in FIG. 28 (tablet cores T2 and T3), FIG. 29 (tablet cores T6 and T8), and FIG. 30 (tablet cores T4 and T5). For all of the mixes tested, the presence of dibasic calcium phosphate anhydrous resulted in a larger increase in dissolution on acid adjustment which suggested an interaction of dibasic calcium phosphate anhydrous with the acalabrutinib maleate. In contrast, no significant increase was observed for the mixes containing no dibasic calcium phosphate anhydrous. Additionally, when binary compacts of dibasic calcium phosphate anhydrous and acalabrutinib maleate were evaluated, those binary compacts showed an increase in the extent of acalabrutinib degradation compared to acalabrutinib maleate alone.

### Example 11: Assessment of Acalabrutinib Maleate Tablet Stability

### A. Tablet T19 Stability

A stability study was conducted to evaluate acalabrutinib maleate tablets (T19) under open storage conditions and when presented in the following three packs:
Bulk Pack: Aluminium foil laminate bag, 4-layer, Tear-off -185 x 280 mm (60 tablets per bag)
HPDE bottle-110 mL induction sealed with 1 g silica gel desiccant cannister (60 tablets per bottle)
HPDE bottle-110 mL induction sealed with 2 g silica gel desiccant cannister (60 tablets per bottle)

The storage conditions investigated in the stability study are detailed in Table 11 below.

TABLE 11

| | |
|---|---|
| CONDITION | FINAL TIME POINT |
| 25° C./60% RH | 156 weeks |
| 30° C./75% RH | 156 weeks |
| Light exposed | 10 days |
| 40° C./75% RH | 26 weeks |
| 30° C./75% RH (open) | 4 weeks |
| 40° C./75% RH (open) | 4 weeks |
| 50° C. | 13 weeks |

As of the 26-week timepoint, the following data was available:
Description: No change in the physical appearance of any of the samples.
Assay: No trends were observed in the assay data in any samples tested.
Organic Impurities:
   ◯ For samples that were stored in appropriate packaging (HDPE bottle with desiccant or aluminum bulk bag) the level of impurities complied with the specification limit of NMT 0.7% for qualified impurities and NMT 0.2% for unqualified impurities.
   ◯ Storage for four weeks exposed to 40° C./75%RH resulted in a level of 4-{2-[(2S)-1-(2-butynoyl)-2-pyrrolidinyl]-5-carbamimidoyl-1H-imidazol-4-yl}-N-(2-pyridinyl)benzamide which was above the specification limit of NMT 0.2%. All other impurities complied with the specification limits of NMT 0.7% for qualified impurities and NMT 0.2% for unqualified impurities.
Enantiomeric purity: All samples met the criteria (>99.6%) of the method at the initial and 26-week timepoint.
Dissolution (0.1 N HCl): No trends were observed in any samples. All samples complied with the specification (Q = 80% at 20 minutes).
Dissolution (pH 6.8): No trends were observed in any samples. All samples complied with Q = 80% at 20 minutes.
Water content: No trends were observed in any of the samples stored with desiccant or in the bulk pack. The open storage samples all showed an increase in water content at 4 weeks with the largest increase being in the 40° C./75% RH sample.
Water activity: No trends in the results were observed.
Microbiological quality: All results complied with the specification (Pharm Eur/USP).

Based on the data generated, an aluminium bulk bag was considered appropriate to ensure an appropriate bulk hold time and an HDPE bottle containing desiccant was considered appropriate to ensure an appropriate shelf life for the acalabrutinib maleate film-coated tablets tested.

### B. Additional Stability Assessments

A stability study was conducted to assess the chemical stability of acalabrutinib maleate tablets T2 and T3 and the following general observations were made:
The presence of dibasic calcium phosphate anhydrous contributed to the formation of 4-{8-Amino-3-[(2S)-2-pyrrolidinyl]-imidazo[1,5-a]-pyrazin-1-yl}-N-(2-pyridinyl)-benzamide and RRT 0.05.
The presence of magnesium stearate contributed to the formation of 4-{2-[(2S)-1-(2-butynoyl)-2-pyrrolidinyl]-5-carbamimidoyl-1H-imidazol-4-yl}-N-(2-pyridinyl)-benzamide and RRT 0.82.
The presence of microcrystalline cellulose contributed to the formation of 4-{3-[(2S)-1-acetoacetyl-2-pyrrolidinyl]-8-aminoimidazo[1,5-a]pyrazin-1-yl}-N-(2-pyridinyl)benzamide, RRT 0.82, and RRT 0.05.

A limited stability study with limited data assessment was performed on acalabrutinib maleate tablets T7 and T15 and the following observations were made:
The main degradation products were 4-{3-[(2S)-l-acetoacetyl-2-pyrrolidinyl]-8-aminoimidazo[1,5-a]pyrazin-1-yl}-N-(2-pyridinyl)benzamide, RRT 0.82, and 4-{2-[(2S)-1-(2-butynoyl)-2-pyrrolidinyl]-5-carbamimidoyl-1H-imidazol-4-yl}-N-(2-pyridinyl)-benzamide.
The increase in the levels of 4-{3-[(2S)-1-acetoacetyl-2-pyrrolidinyl]-8-aminoimidazo[1,5-a]pyrazin-1-yl}-N-(2-pyridinyl)benzamide and RRT 0.82 were greater than observed for acalabrutinib maleate tablets T2 and T3.
Humidity appears to contribute significantly to the formation of RRT 0.82, but likely could be controlled via appropriate packaging.

### Example 12: Preparation of Acalabrutinib Maleate

A. Conversion of Acalabrutinib Free Base to Acalabrutinib Maleate

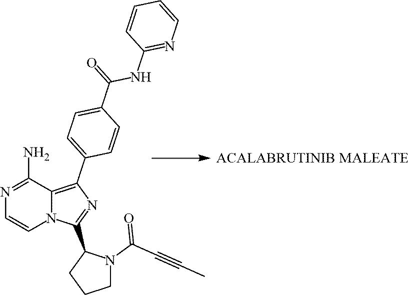

Acalabrutinib (18 kg, 1.0 molar equivalents) in tetrahydrofuran (162 L, 9.0 relative volumes) and water (9 L, 0.5 relative volumes) was heated to 50° C., and filtered. Tetrahydrofuran (9 L, 0.5 relative volumes) was used as a line-wash. Maleic acid (5 kg, 1.1 molar equivalents) in tetrahydrofuran (68 L, 3.75 relative volumes) was added at 50° C., followed by a tetrahydrofuran (5 L, 0.25 relative volumes) line-wash. The mixture was seeded with acalabrutinib maleate (18 mg, 0.001 relative weight), held for 1 hour at 50° C. and then cooled to 20° C. over 1 hour and held for 1 hour, before being circulated through a wet mill to achieve the desired particle size distribution. The product was then filtered and washed with tetrahydrofuran (36 L, 2.0 relative volumes), and then dried by a flow of nitrogen (at >20% relative humidity) at 40° C. to yield acalabrutinib maleate (20.4 kg, 88%) as the monohydrate.

B. Conversion of 4-{8-Amino-3-[(2S)-2-Pyrrolidinyl]Imidazo[1,5-a]Pyrazin-1-yl}-N-(2-Pyridinyl)-Benzamide to Acalabrutinib Maleate

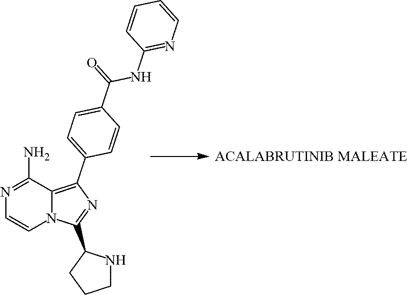

In an alternative method for preparing acalabrutinib maleate, the maleate salt was prepared without the intervening isolation of the acalabrutinib free base. To a mixture of 4-{ 8-amino-3-[(2S)-2-pyrrolidinyl]imidazo[1,5-a]pyrazin-1-yl}-N-(2-pyridinyl)-benzamide (15.0 g, 1.0 molar equivalents) and triethylamine (13.2 mL, 2.6 molar equivalents) in tetrahydrofuran (80 mL, 5.3 relative volumes) was added 2-butynoic acid (3.3 g, 1.1 molar equivalents) in tetrahydrofuran (15 mL, 1.0 relative volume) (over 1 hour), and, after 8 minutes, the propylphosphonic anhydride (53%w/w in ethyl acetate) (23.7 g, 1.1 molar equivalents) in tetrahydrofuran (15 mL, 1.0 relative volumes) was added simultaneously (over 1 hour). The mixture was stirred until the reaction had completed. The mixture was quenched with water (30 mL, 2.0 relative volumes) and the aqueous phase was separated off and discarded. The remaining organic phase was screened through a filter with a line-wash of tetrahydrofuran (7 mL, 0.5 relative volumes). The mixture was then heated to 50° C. and treated with maleic acid (8 g, 1.9 molar equivalents) in tetrahydrofuran (59 mL, 3.9 relative volumes). The mixture was seeded with acalabrutinib maleate (15 mg, 0.001 relative weight), and then cooled to 20° C. over 5 hours, filtered, and washed three times with ethanol (30 mL, 2.0 relative volumes), then tert-butyl methyl ether (58 mL, 3.9 relative volumes), then sucked dry on the filter for 30 minutes to yield acalabrutinib maleate (16 g, 74%) as the monohydrate.

Analysis of the product of Method B above indicated the presence of an impurity, (2Z)-4-[(2S)-2-{8-amino-1-[4-(2-pyridinylcarbamoyl)phenyl]imidazo[1,5-a]pyrazin-3-yl}-1-pyrrolidinyl]-4-oxo-2-butenoic acid, that was not observed in the product of Method A. This impurity was present in an amount that potentially would require impurity toxicity qualification for regulatory registration of an acalabrutinib maleate tablet formulated with drug substance prepared by Method B.

### Example 13: Preparation of Acalabrutinib Maleate Tablets

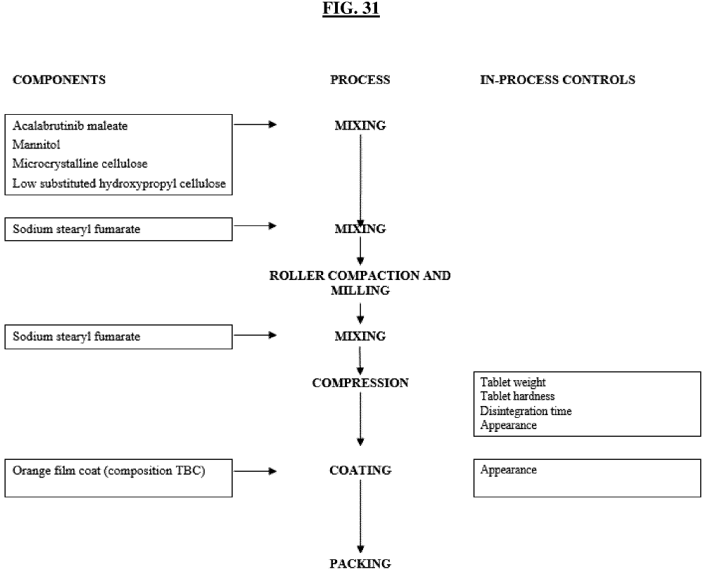

FIG. 31 provides a schematic overview of a process for preparing acalabrutinib maleate tablet T21 of Example 4. Specifically, acalabrutinib maleate, mannitol, microcrystalline cellulose, and low substituted hydroxypropyl cellulose are added to a suitable diffusion mixer and mixed together. The intragranular portion of sodium stearyl fumarate is added to the powders and mixed prior to roller compaction. Ribbons are produced by roller compacting the lubricated blend. Subsequently the ribbons are milled into granules by passing the ribbons through a suitable mill. The granules are mixed with the extragranular portion of sodium stearyl fumarate using a suitable diffusion mixer. The lubricated granules are compressed into tablet cores using a suitable tablet press. The orange film-coating suspension is prepared and applied to the tablet cores using a conventional film coating process.

### Example 14: Relative Bioavailability Study

A Phase 1, open label, single-dose, sequential randomized study of acalabrutinib maleate tablets in healthy human subjects is conducted to evaluate relative bioavailability, proton pump inhibitor (rabeprazole) effect, food effect, and particle size effect. The study is divided into two study parts. Study Part 1 is intended to test the relative bioavailability of the acalabrutinib maleate tablets versus acalabrutinib free base capsules as a pilot study to inform the Study Part 2 design. Study Part 1 is also intended to test the impact of a proton pump inhibitor (“PPI”) and the effect of food on the exposure to acalabrutinib maleate tablets. Following a review of the safety and pharmacokinetic data from Part 1, the study will continue to Study Part 2. Study Part 2 is intended to test the effect of drug substance particle size variants on exposure to acalabrutinib maleate tablets and relative bioavailability of acalabrutinib maleate tablets versus solution. The study results will provide information on the pharmacokinetic and pharmacodynamic profiles of the acalabrutinib maleate tablets to be evaluated.

### A. Study Design

### Part 1 Study Objectives

Primary Objective:
To assess the relative bioavailability of the acalabrutinib maleate tablet compared with the acalabrutinib free base capsule in the fasted state.

Secondary Objectives:
To assess the ACP-5862 pharmacokinetic profile of the acalabrutinib maleate tablet compared with the acalabrutinib free base capsule in fasted state.
To evaluate the effects of the proton pump inhibitor rabeprazole on the pharmacokinetic profiles of acalabrutinib and its metabolite (ACP-5862) obtained after dosing the acalabrutinib maleate tablet.
To evaluate the effect of food on the pharmacokinetics of acalabrutinib and its metabolite (ACP-5862) obtained after dosing the acalabrutinib maleate tablet.
To assess the safety and tolerability of single doses of the acalabrutinib maleate tablet in healthy subjects.
To measure the pharmacodynamic parameter BTK receptor occupancy for acalabrutinib maleate tablet and acalabrutinib free base capsule in isolated PBMCs.

Exploratory Objectives:
To evaluate exposure differences by H pylori breath test status (present vs absent).
To collect SmartPill pH information and use this information as an input to PBPK model to calculate individual in vivo dissolution.

### Part 2 Study Objectives

Primary Objective:
To assess the impact of drug substance particle size on the bioavailability of acalabrutinib maleate tablets.

Secondary Objectives:
To assess the impact of drug substance particle size on the ACP-5862 pharmacokinetic profile of acalabrutinib maleate tablets.
To compare the pharmacokinetics of acalabrutinib maleate tablets versus acalabrutinib oral solution in healthy subjects.
To assess the safety and tolerability of single doses of acalabrutinib maleate tablets with various drug substance particle size distribution in healthy subjects.
To assess the safety, tolerability, taste, and smell of single doses of acalabrutinib oral solution in healthy subjects.

### Part 1 Study Design:

Part 1 of the study is an open-label, three-treatment-period, four-treatment, single-center relative bioavailability, PPI effect, and food-effect randomized crossover study of a new acalabrutinib maleate tablet in healthy subjects (males or females of non-childbearing potential).

Study Part 1 comprises:
A screening period of maximum 28 days;
Three treatment periods during which subjects will be resident from prior to the evening meal the night before dosing (Day -1) until at least 48 hours after dosing and discharged on the morning of Day 3; and
A follow-up visit within 7 to 10 days.

There will be a minimum washout period of 7 days between each acalabrutinib administration. Each subject will receive three of the following four treatments in three treatment periods under fasted or fed conditions: Subjects will be randomized to receive either Treatment A or B in Treatment Periods 1 and 2, followed by either Treatment C or D in Treatment Period 3. The 100 mg acalabrutinib maleate tablet (Variant 1) has the composition of Tablet T21 (see Example 4, Table 7) wherein the drug substance has a D_{(v,0.9)} particle size no greater than 218 pm.
Treatment A: 100 mg acalabrutinib free base capsule, fasted state (> 10 h)*.
Treatment B: 100 mg acalabrutinib maleate tablet (Variant 1), fasted state (>10 h)*.
Treatment C: 100 mg acalabrutinib maleate tablet (Variant 1), fed state*,**.
Treatment D: Rabeprazole 20 mg X 1 (fasted) at 2 hours before administration of 100 mg acalabrutinib maleate tablet (Variant 1)* and following prior administration of rabeprazole 20 mg BID (with meals) on Days -3, -2 and -1.

* For each subject, a SmartPill will be administered with 120 mL of still water followed immediately by a single oral dose of acalabrutinib maleate tablet (Treatment B, C or D) or acalabrutinib free base capsule (Treatment A) administered with 120 mL of still water, followed by PK sampling over 24 hours.
** Subjects will start to consume a high-fat (as per FDA) meal 30 minutes before administration of SmartPill/100 mg acalabrutinib maleate tablet. Subjects will be required to eat the meal in 25 minutes; however, the SmartPill/IMP should be administered 30 minutes after start of the meal.

### Part 2 Study Design:

Part 2 of this study will be an open-label, 4-treatment-period, 4-treatment, single-center relative bioavailability, randomized crossover study to determine the effect of particle size on the PK of a single dose of acalabrutinib maleate tablet in healthy subjects (males or females of non-childbearing potential).

Study Part 2 comprises:
A screening period of maximum 28 days;
Four treatment periods during which subjects will be resident prior to the evening meal the night before dosing (Day -1) until at least 48 hours after dosing and discharged on the morning of Day 3; and
A follow-up visit within 7 to 10 days.

There will be a minimum washout period of at least 3 days between each acalabrutinib administration.

Each subject will receive the following treatments:
Treatment A: 100 mg acalabrutinib maleate tablet (Variant 1), fasted state
Treatment B: 100 mg acalabrutinib maleate tablet (Variant 2), fasted state
Treatment C: 100 mg acalabrutinib maleate tablet (Variant 3), fasted state
Treatment D: 100 mg acalabrutinib solution, fasted state

The 100 mg acalabrutinib maleate tablet (Variant 1) comprises drug substance having an intermediate particle size while the 100 mg acalabrutinib maleate tablet (Variant 2) comprises drug substance having a smaller particle size and the 100 mg acalabrutinib maleate tablet (Variant 3) comprises drug substance having a larger particle size. Specifically, the 100 mg acalabrutinib maleate tablets have the composition of Tablet T21 (see Example 4, Table 7) wherein Variant 1 comprises drug substance having a D_{(v,0.9)} particle size no greater than 218 µm, Variant 2 comprises drug substance having a D_{(v,0.9)} particle size no greater than 160 µm, and Variant 3 comprises drug substance having a D_{(v,0.9)} particle size no greater than 319 µm,

### Expected Duration of the Study

In Part 1, each subject will be involved in the study for approximately 7 to 8 weeks. In Part 2, each subject will be involved in the study for approximately 6 to 7 weeks.

### Targeted Study Population

In Part 1 of the study, a total of 28 healthy male and female subjects aged between 18 to 55 years (inclusive), will be included to ensure at least 24 evaluable subjects. In Part 2 of the study, a total of 24 healthy male and female subjects aged between 18 to 55 years (inclusive) will be included to ensure 20 evaluable subjects at the end of the last treatment period.

### Outcome Endpoints

### Pharmacokinetic Endpoints:

Serial venous blood samples will be obtained for the determination of acalabrutinib and metabolite (ACP-5862) concentrations in plasma. Where possible, pharmacokinetic parameters will be assessed for acalabrutinib and metabolite ACP-5862 on plasma concentrations.

Parts 1 and 2:
Primary PK parameters: Acalabrutinib Cₘₐₓ, AUCₗₐₛₜ, AUC_{inf}
Secondary PK parameters: ACP-5862 Cₘₐₓ, AUCₗₐₛₜ, AUC_{inf}; Acalabrutinib and ACP-5862: AUC₀₋₁₂, AUCₗₐₛₜ, AUC_{inf}, %AUCₑₓₜᵣₐₚ, Cₘₐₓ, t_{½}, tₘₐₓ, Kel, Fᵣₑₗ, CL/F (parent only), Vz/F (parent only), ACP-5862 (metabolite) to acalabrutinib (parent) ratio (M/P) for Cₘₐₓ, AUCₗₐₛₜ, AUC_{inf}.
Additional PK parameters may be determined where appropriate.

### Safety and Tolerability Endpoints:

Safety and tolerability variables will include:
Adverse events/Serious adverse events.
Laboratory assessments (hematology, clinical chemistry, coagulation and urinalysis).
Physical examination.
Electrocardiogram (12-lead ECG).
Vital signs (systolic and diastolic BP, pulse rate, respiratory rate, body temperature).
Taste and smell assessment (only Part 2).

Exploratory Endpoints (Part 1):
Acalabrutinib and ACP-5862: A repeat measures analysis of covariance (ANCOVA) will be used to analyze PK parameters (AUCₗₐₛₜ, AUC_{inf}, and Cₘₐₓ) and to evaluate exposure differences by gastric pH and gastric emptying rate using the appropriate statistical procedure
Whole GI tract temperature, pH and pressure profiles; stomach pH immediately (first measurable point) following dosing of acalabrutinib products (only Part 1)
H pylori breath test status

### Part 1 Statistical Methods

To assess the relative bioavailability of the acalabrutinib maleate tablet compared with acalabrutinib free base capsule in fasted state, the primary PK parameters of acalabrutinib and its metabolite, ACP-5862, will be compared between Treatment B (acalabrutinib) versus A (acalabrutinib free base capsule). The analyses will be performed using a linear mixed-effects analysis of variance model using the natural logarithm of Cₘₐₓ, AUC_{inf}, and AUCₗₐₛₜ as the response variables, sequence, period, treatment as fixed effect and volunteer nested within sequence as random effect. Transformed back from the logarithmic scale, geometric means together with CIs (2-sided 95%) for AUC_{inf}, AUCₗₐₛₜ, and Cₘₐₓ will be estimated and presented. Also, ratios of geometric means together with CIs (2-sided 90%) will be estimated and presented.

To evaluate the effects of proton pump inhibitor rabeprazole on acalabrutinib and its metabolite (ACP-5862) PK profiles obtained after dosing the acalabrutinib maleate tablet, the primary PK parameters of acalabrutinib and its metabolite, ACP-5862, will be compared between Treatment D (rabeprazole) versus B (acalabrutinib), from the same analysis of variance (ANOVA) model.

To evaluate the effect of food on acalabrutinib and its metabolite (ACP-5862) PK obtained after dosing the acalabrutinib maleate tablet, the primary PK parameters of acalabrutinib and its metabolite, ACP-5862, will be compared between Treatment C (fed) versus B (fasted), from the same ANOVA model.

### Part 2 Statistical Methods

To assess the impact of drug substance particle size on the bioavailability of acalabrutinib maleate tablets, the primary PK parameters of acalabrutinib and its metabolite, ACP-5862, will be compared between Treatment B (smaller than target) vs A (target), C (larger than target) vs A (target) and C (larger than target) vs B (smaller than target) and the analyses will be performed using a linear mixed-effects analysis of variance model using the natural logarithm of Cₘₐₓ, AUC_{inf}, and AUCₗₐₛₜ as the response variables, sequence, period, treatment as fixed effect and volunteer nested within sequence as random effect. Transformed back from the logarithmic scale, geometric means together with CIs (2-sided 95%) for AUC_{inf}, AUCₗₐₛₜ, and Cₘₐₓ will be estimated and presented. Also, ratios of geometric means together with CIs (2-sided 90%) will be estimated and presented.

To compare PK of acalabrutinib maleate tablet versus acalabrutinib oral solution, the primary PK parameters of acalabrutinib and its metabolite, ACP-5862, will be compared between treatments D (solution) versus A (target), from the same ANOVA model.

### Part 1 and Part 2 Statistical Methods

Additionally, the 90% CI for the difference in median tmax will be calculated and presented, using the same comparisons from the ANOVAs. The median differences and 90% confidence intervals will be tabulated for each comparison and analyte.

The results are expected to demonstrate that co-administration of PPIs or other acid reducing agents together with acalabrutinib maleate tablets does not affect acalabrutinib and ACP-5862 exposure.

### B. Study Results

### Pharmacokinetics

The Part 1 study results showed that the acalabrutinib maleate tablet (Variant 1) and the acalabrutinib capsule had similar bioavailability.
The mean pharmacokinetic exposures (Cₘₐₓ and AUCs) of acalabrutinib and metabolite ACP-5862 were similar following oral administration of acalabrutinib maleate tablet (Variant 1) versus acalabrutinib capsule under fasted state. Relative bioavailability was approximately 91% and 98% for acalabrutinib Cₘₐₓ and AUCs, respectively, and was approximately 100% and 103% to 104% for ACP-5862 Cₘₐₓ and AUCs, respectively.
Co-administration of PPI (rabeprazole) with acalabrutinib maleate tablet (Variant 1) had no apparent effect on the pharmacokinetic exposures of acalabrutinib and metabolite ACP-5862. Cₘₐₓ was slightly lower (-24% difference in geometric means) and AUCs were slightly higher (~14 to 17% difference in geometric means) for acalabrutinib. Cₘₐₓ of ACP-5862 was approximately 30% lower, with comparable AUCs in the presence versus absence of PPI.
For acalabrutinib maleate tablet (Variant 1), food reduced the Cₘₐₓ of acalabrutinib and ACP-5862 by approximately 54% and 36%, respectively, and had no effect on overall AUCs.
As there was no differences in BTK occupancy across treatments, and the between-subject variability (geometric CV%) in Cₘₐₓ of acalabrutinib and ACP-5862 was up to approximately 81%, the observed differences in Cₘₐₓ are unlikely to have a clinically meaningful impact.

Summaries of plasma pharmacokinetic parameters from Part 1 of the study are presented in Tables 12-16 below.

TABLE 12

Part 1: Summary of Plasma Pharmacokinetic Parameters for Acalabrutinib

| Parameter (Unit) | Statistics | A (N=30) | B (N=29) | C (N=14) | D (N=14) |
|---|---|---|---|---|---|
| Cmax (ng/mL) | Geometric Mean | 541.6 | 504.9 | 255.6 | 371.9 |
| | Geometric CV % | 41.06 | 49.88 | 46.52 | 81.44 |
| AUCinf (h.ng/mL) | Geometric Mean | 569.9 | 559.5 | 528.7 | 694.1 |
| | Geometric CV % | 25.55 | 34.63 | 18.20 | 39.74 |
| AUClast (h·ng/mL) | Geometric Mean | 565.7 | 556.2 | 525.7 | 669.7 |
| | Geometric CV % | 25.41 | 34.73 | 18.34 | 40.54 |
| tmax (h) | Median | 0.75 | 0.73 | 2.00 | 1.01 |
| | Min, Max | 0.48, 2.02 | 0.25, 1.53 | 0.25, 4.00 | 0.23, 3.00 |
| t½λz (h) | Mean | 1.954 | 1.515 | 1.347 | 2.863 |
| | SD | 2.031 | 0.6280 | 0.3991 | 2.313 |
| CL/F (L/h) | Mean | 180.9 | 188.7 | 192.0 | 154.4 |
| | SD | 46.45 | 64.41 | 34.22 | 61.75 |
| Vz/F (L) | Mean | 469.3 | 386.8 | 373.9 | 583.6 |
| | SD | 332.5 | 135.9 | 134.8 | 456.9 |

A: 100 mg acalabrutinib capsule, fasted state
B: 100 mg acalabrutinib maleate tablet (Variant 1), fasted state
C: 100 mg acalabrutinib maleate tablet (Variant 1), fed state
D: 20 mg Rabeprazole QD (fasted) at 2 hours before administration of 100 mg acalabrutinib maleate tablet (Variant 1) and following prior administration of 20 mg rabeprazole BID (with meals) on Days -3, -2, and -1.
BID = twice daily; CV = coefficient of variation; Max = maximum; Min = minimum; N=number of subjects in the PK analysis set; QD = once daily; SD = standard deviation.

TABLE 13

Part 1: Summary of Plasma Pharmacokinetic Parameters for Metabolite ACP-5862

| Parameter (Unit) | Statistics | A (N=30) | B (N=29) | C (N=14) | D (N=14) |
|---|---|---|---|---|---|
| Cmax (ng/mL) | Geometric Mean | 533.7 | 538.5 | 358.4 | 365.3 |
| | Geometric CV% | 37.21 | 42.19 | 33.19 | 56.45 |
| AUCinf (h·ng/mL) | Geometric Mean | 1625 | 1672 | 1644 | 1783 |
| | Geometric CV% | 24.14 | 24.84 | 17.13 | 28.71 |
| AUClast (h·ng/mL) | Geometric Mean | 1534 | 1575 | 1532 | 1656 |
| | Geometric CV% | 24.58 | 25.87 | 17.36 | 29.58 |
| tmax (h) | Median | 1.00 | 0.80 | 2.98 | 1.75 |
| | Min, Max | 0.73, 3.00 | 0.50, 2.98 | 0.98, 4.02 | 0.52, 6.00 |
| t½λz (h) | Mean | 7.824 | 8.152 | 7.945 | 7.848 |
| | SD | 1.558 | 1.287 | 1.439 | 1.579 |
| M:P [AUC] | Geometric Mean | 2.756 | 2.890 | 3.007 | 2.484 |
| | Geometric CV% | 21.06 | 25.32 | 17.89 | 22.32 |
| M:P [Cmax] | Geometric Mean | 0.9526 | 1.031 | 1.356 | 0.9496 |
| | Geometric CV% | 27.67 | 32.12 | 30.17 | 37.94 |

A: 100 mg acalabrutinib capsule, fasted state
B: 100 mg acalabrutinib maleate tablet (Variant 1), fasted state
C: 100 mg acalabrutinib maleate tablet (Variant 1), fed state
D: 20 mg rabeprazole QD (fasted) at 2 hours before administration of 100 mg acalabrutinib maleate tablet (Variant 1) and following prior administration of 20 mg rabeprazole BID (with meals) on Days -3, -2, and -1.
BID = twice daily; CV = coefficient of variation; Max = maximum; Min = minimum; N=number of subjects in the PK analysis set; QD = once daily; SD = standard deviation.

TABLE 14

Part 1: Statistical Comparisons of Pharmacokinetic Parameters for Assessment of Relative Bioavailability

| Analyte | Parameter (unit) | Treatment | N | n | Geometric LSM | 95% CI | Pairwise Comparison (B/A) | | | |
|---|---|---|---|---|---|---|---|---|---|---|
| | | | | | | | Ratio (%) | 90% CI | Inter-CV% | Intra-CV% |
| Acalabrutinib | Cmax (ng/mL) | A | 30 | 29 | 556.2 | (471.7, 655.7) | 90.50 | (79.42, 103.1) | 33.5 | 29.8 |
| | | B | 29 | | 503.3 | (426.9, 593.5) | | | | |
| | AUCinf (h·ng/mL) | A | 30 | 29 | 571.4 | (510.1, 640.3) | 97.90 | (91.62, 104.6) | 26.6 | 14.9 |
| | | B | 29 | | 559.4 | (499.2, 626.8) | | | | |
| | AUClast (h·ng/mL) | A | 30 | 29 | 567.2 | (506.1, 635.6) | 98.05 | (91.77, 104.8) | 26.6 | 14.9 |
| | | B | 29 | | 556.1 | (496.2, 623.2) | | | | |
| ACP-5862 | Cmax (ng/mL) | A | 30 | 29 | 537.4 | (463.4, 623.2) | 99.84 | (91.94, 108.4) | 35.5 | 18.6 |
| | | B | 29 | | 536.5 | (462.6, 622.2) | | | | |
| | AUCinf (h·ng/mL) | A | 30 | 29 | 1615 | (1472, 1773) | 103.6 | (100.1, 107.3) | 23.5 | 7.8 |
| | | B | 29 | | 1673 | (1525, 1837) | | | | |
| | AUClast (h·ng/mL) | A | 30 | 29 | 1525 | (1385, 1679) | 103.3 | (99.86, 106.9) | 24.4 | 7.6 |
| | | B | 29 | | 1576 | (1431, 1735) | | | | |

A: 100 mg acalabrutinib capsule, fasted state
B: 100 mg acalabrutinib maleate tablet (Variant 1), fasted state.
Only the subjects with valid PK parameter in both treatments are included for statistical analysis.
Result based on linear mixed effect ANOVA of log-transformed PK parameter with sequence, period, treatment as fixed effect, and subject nested within sequence as random effect. Geometric mean ratio and corresponding 90% CI are back-transformed and presented as percentages. Geometric LSM and corresponding 95% CI are also back transformed. ANOVA = Analysis of variance; CI = Confidence interval; LSM = Least-squares mean; N = Number of subjects in the PK analysis set; n = Number of subjects included in the statistical comparison analysis; PK = Pharmacokinetics.

TABLE 15

Part 1: Statistical Comparisons of Pharmacokinetic Parameters for Assessment of PPI Effect

| Analyte | Parameter (Unit) | Treatment | N | n | Geometric LSM | 95% CI | Pairwise Comparison (D/B) | |
|---|---|---|---|---|---|---|---|---|
| | | | | | | | Ratio (%) | 90% CI |
| Acalabrutinib | Cmax (ng/mL) | B | 29 | 14 | 486.9 | (338.2, 700.9) | 76.39 | (54.88, 106.3) |
| | | D | 14 | | 371.9 | (258.3, 535.5) | | |
| | AUCinf (h·ng/mL) | B | 29 | 14 | 591.1 | (466.9, 748.4) | 117.4 | (105.4, 130.8) |
| | | D | 14 | | 694.1 | (548.2, 878.7) | | |
| | AUClast (h·ng/mL) | B | 29 | 14 | 587.8 | (462.6, 746.9) | 113.9 | (101.4, 128.0) |
| | | D | 14 | | 669.7 | (527.0, 850.9) | | |
| ACP-5862 | Cmax (ng/mL) | B | 29 | 14 | 523.6 | (390.7, 701.9) | 69.76 | (51.31, 94.86) |
| | | D | 14 | | 365.3 | (272.5, 489.6) | | |
| | AUCinf (h·ng/mL) | B | 29 | 14 | 1770 | (1491, 2101) | 100.7 | (93.26, 108.8) |
| | | D | 14 | | 1783 | (1502, 2117) | | |
| | AUClast (h·ng/mL) | B | 29 | 14 | 1666 | (1397, 1985) | 99.40 | (90.81, 108.8) |
| | | D | 14 | | 1656 | (1389, 1973) | | |

B: 100 mg acalabrutinib maleate tablet (Variant 1), fasted state
D: 20 mg rabeprazole QD (fasted) at 2 hours before administration of 100 mg acalabrutinib maleate tablet (Variant 1) and following prior administration of
20 mg rabeprazole BID (with meals) on Days -3, -2, and -1.
Only the subjects with valid PK parameters from both treatments were included for statistical analysis.
Result based on linear mixed effect ANOVA of log-transformed PK parameter with sequence, treatment as fixed effect; and subject nested within sequence as random effect. Geometric mean ratio and corresponding 90% CI are back-transformed and presented as percentages. Geometric LSM and corresponding 95% CI are also back transformed. ANOVA = analysis of variance; BID = twice daily; CI = confidence interval; LSM = least-squares mean; N = number of subjects in the PK analysis set; n = number of subjects included in the statistical comparison analysis; PK = Pharmacokinetics; QD = once daily.

TABLE 16

Part 1: Statistical Comparisons of Pharmacokinetic Parameters for Assessment of Food Effect

| Analyte | Parameter (Unit) | Treatment | N | n | Geometric LSM | 95% CI | Pairwise Comparison (C/B) | |
|---|---|---|---|---|---|---|---|---|
| | | | | | | | Ratio (%) | 90% CI |
| Acalabrutinib | Cmax | B | 29 | | 555.4 | (446.2, 691.4) | | |
| | (ng/mL) | C | 14 | 14 | 255.6 | (205.3, 318.1) | 46.01 | (35.92, 58.95) |
| | AUCinf | B | 29 | | 541.2 | (483.0, 606.5) | | |
| | (h·ng/mL) | C | 14 | 14 | 528.7 | (471.9, 592.5) | 97.69 | (87.19,109.5) |
| | AUClast | B | 29 | | 538.2 | (480.0, 603.4) | | |
| | (h·ng/mL) | C | 14 | 14 | 525.7 | (468.9, 589.4) | 97.69 | (87.18, 109.5) |
| ACP-5862 | Cmax | B | 29 | | 560.6 358.4 | (469.4, 669.5) | | |
| | (ng/mL) | C | 14 | 14 | | (300.1, 428.1) | 63.94 | (54.15, 75.49) |
| | AUCinf | B | 29 | | 1617 | (1470, 1778) | | |
| | (h·ng/mL) | C | 14 | 14 | 1644 | (1495, 1809) | 101.7 | (96.90, 106.7) |
| | AUClast | B | 29 | | 1531 | (1387, 1691) | | |
| | (h·ng/mL) | C | 14 | 14 | 1532 | (1388, 1692) | 100.1 | (95.47, 104.9) |

B: 100 mg acalabrutinib maleate tablet (Variant 1), fasted state
C: 100 mg acalabrutinib maleate tablet (Variant 1), fed state.
Only the subjects with valid PK parameters from both treatments were included for statistical analysis.
Result based on linear mixed effect ANOVA of log-transformed PK parameter with sequence, treatment as fixed effect; and subject nested within sequence as random effect. Geometric mean ratio and corresponding 90% CI are back-transformed and presented as percentages. Geometric LSM and corresponding 95% CI are also back transformed. ANOVA = analysis of variance; CI = confidence interval; LSM = least-squares mean; N = number of subjects in the PK analysis set; n = number of subjects included in the statistical comparison analysis;
PK = Pharmacokinetics.

The Part 2 study results showed that acalabrutinib maleate particle size had no significant effect on the pharmacokinetics of acalabrutinib and ACP-5862 over the particle size range evaluated. Following administration, Variants 1, 2, and 3 all resulted in comparable pharmacokinetic exposures.
The mean pharmacokinetic exposures (Cₘₐₓ and AUCs) of acalabrutinib and metabolite ACP-5862 were similar following oral administration of acalabrutinib maleate tablet with different particle sizes (Variants 1, 2, and 3). 90% CIs for geometric mean ratios were nearly or well within the 80% to 125% margin.
Acalabrutinib solution had a higher Cₘₐₓ and comparable AUCs versus acalabrutinib maleate tablet (Variant 1). Relative bioavailability was approximately 122% and 102% for acalabrutinib Cₘₐₓ and AUCs, respectively, and was approximately 124% and 106% to 107% for ACP-5862 Cₘₐₓ and AUCs, respectively.

Summaries of plasma pharmacokinetic parameters from Part 2 of the study are presented in Tables 17-21.

TABLE 17

Part 2: Summary of Plasma Pharmacokinetic Parameters for Acalabrutinib

| Parameter (Unit) | Statistics | A (N=24) | B (N=24) | C (N=24)a | D (N=24) |
|---|---|---|---|---|---|
| Cmax (ng/mL) | Geometric Mean | 596.5 | 543.3 | 602.2 | 727.2 |
| | Geometric CV % | 44.29 | 56.12 | 60.51 | 44.90 |
| AUCinf (h·ng/mL) | Geometric Mean | 667.2 | 616.1 | 632.7 | 677.0 |
| | Geometric CV % | 32.91 | 33.01 | 30.00 | 36.84 |
| AUClast (h·ng/mL) | Geometric Mean | 662.9 | 612.8 | 628.7 | 673.5 |
| | Geometric CV % | 33.27 | 33.21 | 30.14 | 36.99 |
| tmax (h) | Median | 0.50 | 0.75 | 0.50 | 0.50 |
| | Min, Max | 0.25, 1.50 | 0.25, 3.00 | 0.25, 2.00 | 0.25, 1.00 |
| t½λz (h) | Mean | 1.768 | 1.364 | 1.794 | 1.685 |
| | SD | 0.7369 | 0.4352 | 0.7305 | 0.7755 |
| CL/F (L/h) | Mean | 157.4 | 170.4 | 164.1 | 156.9 |
| | SD | 49.95 | 54.44 | 42.61 | 57.76 |
| Vz/F (L) | Mean | 400.8 | 334.8 | 414.6 | 378.8 |
| | SD | 245.8 | 193.1 | 191.8 | 222.5 |

^{f} Data from 23 subjects are included in summary; one subject had entire concentrations non-quantifiable, thus no PK parameters were calculable.
A: 100 mg acalabrutinib maleate tablet (Variant 1), fasted state
B: 100 mg acalabrutinib maleate tablet (Variant 2), fasted state
C: 100 mg acalabrutinib maleate tablet (Variant 3), fasted state
D: 100 mg acalabrutinib solution, fasted state.
CV = coefficient of variation; Max = maximum; Min = minimum; N=number of subjects in the pharmacokinetic analysis set; PK = Pharmacokinetics; SD = standard deviation.

TABLE 18

Part 2: Summary of Plasma Pharmacokinetic Parameters for Metabolite ACP-5862

| Parameter (Unit) | Statistics | A (N=24) | B (N=24) | C (N=24)a | D (N=24) |
|---|---|---|---|---|---|
| Cmax (ng/mL) | Geometric Mean | 536.5 | 532.8 | 594.5 | 662.6 |
| | Geometric CV % | 39.38 | 33.25 | 33.65 | 26.54 |
| AUCinf (h·ng/mL) | Geometric Mean | 1746 | 1775 | 1792 | 1845 |
| | Geometric CV % | 24.46 | 24.80 | 25.78 | 26.79 |
| AUClast (h·ng/mL) | Geometric Mean | 1653 | 1674 | 1702 | 1762 |
| | Geometric CV % | 24.97 | 25.69 | 26.03 | 27.08 |
| tmax (h) | Median | 0.77 | 0.88 | 0.75 | 0.74 |
| | Min, Max | 0.48, 2.02 | 0.50, 3.00 | 0.48, 3.50 | 0.48, 1.52 |
| t½λz (h) | Mean | 7.668 | 7.856 | 7.406 | 7.130 |
| | SD | 1.035 | 1.755 | 1.005 | 1.145 |
| M:P [AUC] | Geometric Mean | 2.529 | 2.785 | 2.738 | 2.635 |
| | Geometric CV % | 25.04 | 24.62 | 25.00 | 23.30 |
| M:P [Cmax] | Geometric Mean | 0.8695 | 0.9480 | 0.9544 | 0.8809 |
| | Geometric CV % | 29.87 | 39.18 | 36.26 | 25.13 |

^{g} Data from 23 subjects are included in summary; one subject had entire concentrations non-quantifiable, thus no PK parameters were calculable.
A: 100 mg acalabrutinib maleate tablet (Variant 1), fasted state
B: 100 mg acalabrutinib maleate tablet (Variant 2), fasted state
C: 100 mg acalabrutinib maleate tablet (Variant 3), fasted state; D: 100 mg acalabrutinib solution, fasted state.
CV = coefficient of variation; Max = maximum; Min = minimum; N=number of subjects in the pharmacokinetic analysis set; PK = Pharmacokinetic; SD = standard deviation.

TABLE 19

Part 2: Statistical Comparisons of Acalabrutinib Pharmacokinetic Parameters for Assessment of Particle Size Effect

| Parameter (unit) | Treatment | N | n | Geometric LSM | 95% CI | Pairwise Comparisons | | |
|---|---|---|---|---|---|---|---|---|
| | | | | | | Pair | Ratio (%) | 90% CI |
| Cmax (ng/mL) | A | 24 | | 596.5 | (489.9, 726.4) | | | |
| | B | 24 | 24 | 543.3 | (446.2, 661.6) | B/A | 91.08 | (79.35, 104.5) |
| | A | 24 | | 599.0 | (484.5, 740.5) | | | |
| | C | 24 | 23 | 600.1 | (485.5, 741.9) | C/A | 100.2 | (82.95, 121.0) |
| | B | 24 | | 536.7 | (425.8, 676.4) | | | |
| | C | 24 | 23 | 599.7 | (475.9, 755.9) | C/B | 111.8 | (90.67, 137.7) |
| AUCinf (h·ng/mL) | A | 24 | | 667.2 | (583.4, 763.0) | | | |
| | B | 24 | 24 | 616.1 | (538.7, 704.6) | B/A | 92.35 | (85.64, 99.58) |
| | A | 24 | | 674.5 | (590.3, 770.8) | | | |
| | C | 24 | 23 | 631.9 | (553.0, 722.1) | C/A | 93.68 | (85.37, 102.8) |
| | B | 24 | | 622.1 | (545.6, 709.2) | | | |
| | C | 24 | 23 | 630.7 | (553.2, 719.0) | C/B | 101.4 | (94.20, 109.1) |
| AUClast (h·ng/mL) | A | 24 | | 662.9 | (579.0, 758.9) | | | |
| | B | 24 | 24 | 612.8 | (535.3, 701.7) | B/A | 92.46 | (85.72, 99.72) |
| | A | 24 | | 670.1 | (585.8, 766.6) | | | |
| | C | 24 | 23 | 627.9 | (548.9, 718.3) | C/A | 93.71 | (85.35, 102.9) |
| | B | 24 | | 618.8 | (542.3, 706.0) | | | |
| | C | 24 | 23 | 626.7 | (549.3, 715.0) | C/B | 101.3 | (94.08, 109.1) |

A: 100 mg acalabrutinib maleate tablet (Variant 1), fasted state
B: 100 mg acalabrutinib maleate tablet (Variant 2), fasted state
C: 100 mg acalabrutinib maleate tablet (Variant 3), fasted state.
One subject for Treatment C had entire concentrations non-quantifiable, thus no PK parameters were calculable.
Result based on linear mixed effect ANOVA of log-transformed PK parameter with sequence, period, treatment as fixed effect; and subject nested within sequence as random effect. Geometric mean ratio and corresponding 90% CI are back-transformed and presented as percentages. Geometric LSM and corresponding 95% CI are also back transformed. ANOVA = analysis of variance; CI = confidence interval; LSM = least-squares mean; N = Number of subjects in the PK analysis set; n = number of subjects included in the statistical comparison analysis; PK = Pharmacokinetics.

TABLE 20

Part 2: Statistical Comparisons of ACP-5862 Pharmacokinetic Parameters for Assessment of Particle Size Effect

| Parameter (unit) | Treatment | N | n | Geometric LSM | 95% CI | Pairwise Comparisons | | |
|---|---|---|---|---|---|---|---|---|
| | | | | | | Pair | Ratio (%) | 90% CI |
| Cmax (ng/mL) | A | 24 | | 536.5 | (463.1, 621.6) | | | |
| | B | 24 | 24 | 532.8 | (459.9, 617.2) | B/A | 99.30 | (88.73, 111.1) |
| | A | 24 | | 537.7 | (461.5, 626.5) | | | |
| | C | 24 | 23 | 593.0 | (509.0, 691.0) | C/A | 110.3 | (97.02, 125.4) |
| | B | 24 | | 528.5 | (457.9, 609.8) | | | |
| | C | 24 | 23 | 594.2 | (514.9, 685.7) | C/B | 112.4 | (103.1, 122.7) |
| AUCinf (h·ng/mL) | A | 24 | | 1746 | (1574, 1936) | | | |
| | B | 24 | 24 | 1775 | (1600, 1968) | B/A | 101.7 | (98.39 105.0) |
| | A | 24 | | 1746 | (1566, 1946) | | | |
| | C | 24 | 23 | 1788 | (1604, 1993) | C/A | 102.4 | (98.78, 106.2) |
| | B | 24 | | 1774 | (1589, 1980) | | | |
| | C | 24 | 23 | 1789 | (1603, 1997) | C/B | 100.9 | (97.11, 104.8) |
| AUClast (h·ng/mL) | A | 24 | | 1653 | (1486, 1839) | | | |
| | B | 24 | 24 | 1674 | (1505, 1862) | B/A | 101.3 | (97.60, 105.1) |
| | A | 24 | | 1653 | (1481, 1845) | | | |
| | C | 24 | 23 | 1699 | (1522, 1896) | C/A | 102.8 | (98.74, 107.0) |
| | B | 24 | | 1673 | (1495, 1872) | | | |
| | C | 24 | 23 | 1700 | (1519, 1902) | C/B | 101.6 | (97.52, 105.9) |

A: 100 mg acalabrutinib maleate tablet (Variant 1), fasted state
B: 100 mg acalabrutinib maleate tablet (Variant 2), fasted state
C: 100 mg acalabrutinib maleate tablet (Variant 3), fasted state.
One subject for Treatment C had entire concentrations non-quantifiable, thus no PK parameters were calculable.
Result based on linear mixed effect ANOVA of log-transformed PK parameter with sequence, period, treatment as fixed effect; and subject nested within sequence as random effect. Geometric mean ratio and corresponding 90% CI are back-transformed and presented as percentages. Geometric LSM and corresponding 95% CI are also back transformed. ANOVA = analysis of variance; CI = confidence interval; LSM = least-squares mean; N = Number of subjects in the PK analysis set; n = number of subjects included in the statistical comparison analysis; PK = Pharmacokinetic.

TABLE 21

Part 2: Statistical Comparisons of Pharmacokinetic Parameters for Assessment of Relative Bioavailability

| Analyte | Parameter (Unit) | Treatment | N | n | Geometric LSM | 95% CI | Pairwise Comparison (D/A) | |
|---|---|---|---|---|---|---|---|---|
| | | | | | | | Ratio (%) | 90% CI |
| Acalabrutinib | Cmax | A | 24 | | 596.5 | (501.3, 710.0) | | |
| | (ng/mL) | D | 24 | 24 | 727.2 | (611.1, 865.5) | 121.9 | (106.7, 139.3) |
| | AUCinf | A | 24 | | 667.2 | (577.2, 771.2) | | |
| | (h·ng/mL) | D | 24 | 24 | 677.0 | (585.7, 782.6) | 101.5 | (95.30, 108.1) |
| | AUClast | A | 24 | | 662.9 | (572.9, 766.9) | | |
| | (h·ng/mL) | D | 24 | 24 | 673.5 | (582.1, 779.2) | 101.6 | (95.32, 108.3) |
| ACP-5862 | Cmax | A | 24 | | 536.5 | (469.3, 613.4) | | |
| | (ng/mL) | D | 24 | 24 | 662.6 | (579.6, 757.6) | 123.5 | (109.9, 138.8) |
| | AUCinf | A | 24 | | 1746 | (1567, 1944) | | |
| | (h·ng/mL) | D | 24 | 24 | 1845 | (1657,2055) | 105.7 | (102.7, 108.8) |
| | AUClast | A | 24 | | 1653 | (1482, 1844) | | |
| | (h·ng/mL) | D | 24 | 24 | 1762 | (1580, 1966) | 106.6 | (103.2, 110.2) |

A: 100 mg acalabrutinib maleate tablet (Variant 1), fasted state
D: 100 mg acalabrutinib solution, fasted state.
Result based on linear mixed effect ANOVA of log-transformed PK parameter with sequence, treatment as fixed effect; and subject nested within sequence as random effect. Geometric mean ratio and corresponding 90% CI are back-transformed and presented as percentages. Geometric LSM and corresponding 95% CI are also back transformed. ANOVA = analysis of variance; CI = confidence interval; LSM = least-squares mean; N = number of subjects in the PK analysis set; n = number of subjects included in the statistical comparison analysis; PK = Pharmacokinetics.

### Pharmacodynamics

Part 1 of the study investigated the BTK receptor occupancy of acalabrutinib when administered as a capsule or tablet. Results showed there was similar BTK occupancy across all post-dose time points (4, 12, and 24 hours) following the tablet and capsule administration. In addition, BTK occupancy of the tablet formulation was not affected by administration of food or PPI.

### Exploratory

The stomach pH was found not to influence the exposure to acalabrutinib maleate from the 100 mg acalabrutinib maleate film-coated tablets and therefore the in vivo dissolution of the tablets was not sensitive to stomach pH.

### Safety

Overall, no new safety concerns were found with the 100 mg acalabrutinib maleate film-coated tablets and the new formulation was tolerated well.

### Example 15: Assessment of Bioequivalence

An open-label, randomized, two-way crossover bioequivalence study in healthy subjects is conducted to evaluate bioequivalence of the acalabrutinib maleate tablet (test formulation) and the acalabrutinib free base capsule (reference formulation). The study is intended to demonstrate, in accordance with regulatory requirements, that the acalabrutinib maleate tablet and the acalabrutinib free base capsule are bioequivalent.

### Title of the Study:

A Phase I, Open-Label, Randomized, 2-Treatment, 2-Period, Crossover Study in Healthy Subjects to Assess the Bioequivalence of Acalabrutinib Tablet and Acalabrutinib Capsule.

### Study Rationale:

Acalabrutinib is a Biopharmaceutical Classification System (BCS) class II drug (high permeability, low solubility), which displays two basic dissociation constants in the physiological pH range. The solubility of acalabrutinib is reduced with increasing pH. Below pH 4, the drug is highly soluble. In patients taking acid-reducing agents (i.e., pH above 4), however, drug solubility in the stomach/intestine is insufficient to ensure full drug solubilization and absorption. Previous observations from a Phase I study (Study ACE-HV-112) showed that when acalabrutinib capsule 100 mg is administered following once daily (qd) dosing of 40 mg omeprazole (a proton-pump inhibitor (PPI)), there is a 43% reduction in AUC and a 72% reduction in Cₘₐₓ as compared to when the drug is dosed in normal acidic pH conditions.

At a dose of 100 mg equivalent free moiety, the acalabrutinib maleate tablet (AMT)) shows pH independent release in vitro in contrast to the acalabrutinib capsule (i.e., Calquence). The results from the relative bioavailability study (see Example 14) have demonstrated that the systemic exposures of acalabrutinib and its active metabolite, ACP-5862 following administration of the AMT are similar in the presence or absence of PPIs, and comparable to those observed with the 100 mg acalabrutinib capsule. This bioequivalence study is intended to confirm that the 100 mg AMT eliminates the impact of PPI on the pharmacokinetics (PK) of acalabrutinib in humans.

### Number of Subjects Planned

Approximately 64 subjects (around 32 per treatment sequence) will be randomized to ensure at least 52 evaluable subjects (26 per sequence) at the end of Treatment Period 2.

### Study Objectives

### Primary Objective:

To demonstrate the bioequivalence of AMT and acalabrutinib capsule, administered in the fasted state.

### Secondary Objectives:

To compare the pharmacokinetic profile of ACP-5862, the active metabolite of acalabrutinib, following administration of AMT and acalabrutinib capsule.
To compare the safety and tolerability of single doses of AMT and acalabrutinib capsule.

### Exploratory Objective:

To measure the pharmacodynamics (PD) of acalabrutinib.

### Study Design

This study will be a multicenter, Phase I, open-label, randomized, 2-sequence, 2-treatment, 2-period, crossover, bioequivalence study with single doses of acalabrutinib administered orally in healthy subjects at approximately three study centers in the United States. The study is designed to demonstrate the bioequivalence of AMT (Treatment A) compared with marketed acalabrutinib capsule (Treatment B) in the fasted state.

The study will comprise:
Visit 1: A screening period of up to 28 days before first dosing.
Visit 2: Two treatment periods:
   Subjects will be admitted to the study center on Day -2 of Treatment Period 1 to confirm eligibility before first dosing. Eligibility criteria will be reconfirmed on Day -1 of each treatment period.
   On Day 1 of Treatment Periods 1 and 2, subjects will be administered the assigned treatment (A or B) as randomized, followed by a washout of at least five days between Treatment Periods 1 and 2.
   Subjects will be discharged from the study center on the morning of Day 3 of Treatment Period 2 after the scheduled study assessments have been completed.
Visit 3: A Follow-up Visit/Early Termination Visit at 7 to 10 days after last administration of IMP.

At the Follow-up Visit/Early Termination Visit, a telemedicine visit may replace the on-site visit or parts thereof, if necessary (when a telemedicine visit is conducted, laboratory testing, ECGs, and tympanic temperature will not be performed). The term telemedicine visit refers to virtual or video visits. During a civil crisis, natural disaster, or public health crisis, such as the COVID-19 pandemic, on-site visits may be replaced by a telemedicine visit if allowed by local/regional guidelines. Having a telemedicine contact with the subjects will allow adverse events (AEs) and concomitant medication to be collected according to study requirements to be reported and documented.

Subjects will be randomized to receive either treatment sequence 1 (AB) or treatment sequence 2 (BA). The AMT has the composition of Tablet T21 (see Example 4, Table 7) wherein the drug substance has a D_{(v,0.9)} particle size no greater than 218 pm.
Treatment A: AMT, 100 mg, fasted state.
Treatment B: Acalabrutinib capsule, 100 mg, fasted state.
Subjects will receive fixed single doses of acalabrutinib on two occasions, under fasted conditions.

### Expected Duration of the Study

Each subject will be involved in the study for approximately six weeks.

### Targeted Study Population

Healthy adult male and female subjects aged 18 to 55 years (inclusive), BMI between 18.5 and 30 kg/m2 inclusive, non-smokers; females must be of non-childbearing potential.

Test and Reference Formulations

| | | |
|---|---|---|
| | Test Formulation | Reference Formulation |
| Formulation: | Acalabrutinib maleate tablet (Tablet T21) | Acalabrutinib capsule (commercially available) |
| S trength/concentration: | 100 mg | 100 mg |
| Dose: | 100 mg (100 mg of freebase equivalent) | 100 mg |
| Route of administration: | Oral | Oral |
| Regimen: | Single dose | Single dose |

### Outcome Endpoints

### Safety and Tolerability Endpoints:

Adverse events.
Laboratory assessments (hematology, coagulation, clinical chemistry, and urinalysis).
Physical examination.
Electrocardiogram (ECG).
Vital signs (systolic blood pressure [BP], diastolic BP, pulse, respiratory rate, tympanic, temperature).

### Pharmacokinetic Endpoints:

### Primary PK Parameters:

Acalabrutinib - AUC_{inf}, AUCₗₐₛₜ, Cₘₐₓ

### Secondary PK Parameters:

Acalabrutinib - tₘₐₓ, t_{½} λz, MRT, λz, CL/F, Vz/F
ACP-5862 - AUC_{inf}, AUCₗₐₛₜ, Cₘₐₓ, tₘₐₓ, t_{½}λz, MRT, λz, M:P[AUC], M:P[Cmax]

### Statistical Methods

All statistical analyses and production of tables, figures and listings will be performed using SAS® Version 9.4 or newer version.

### Analysis Data Sets:

The safety analysis set will include all subjects who received at least one dose in Treatment Period 1 and for whom any post-dose safety data are available. The PK analysis set will consist of all subjects in the safety analysis set who have at least one quantifiable post-dose acalabrutinib concentration with no important protocol deviations or adverse events considered to impact the analysis of the PK data. The randomized set will consist of all subjects randomized into the study.

### Presentation and Analysis of Safety and Tolerability Data:

All safety data (scheduled and unscheduled) will be presented in the data listings. Continuous variables will be summarized using descriptive statistics (number of subjects [n], mean, standard deviation [SD], minimum, median, maximum) by treatment. Categorical variables will be summarized in frequency tables (frequency and proportion) by treatment. The analysis of the safety variables will be based on the safety analysis set.

Adverse events will be summarized by system organ class (SOC) and Preferred Term using the current version of Medical Dictionary for Regulatory Activities (MedDRA) vocabulary. Tabulations and listings of data will be presented for vital signs, clinical laboratory tests, and ECGs. Any new or aggravated clinically relevant abnormal medical physical examination finding compared to the baseline assessment will be reported as an adverse event. Clinical laboratory data will be reported in the units provided by the clinical laboratory, and in Système International units.

### Presentation of Pharmacokinetic Data:

Listings of PK blood sample collection times, as well as derived sampling time deviations will be provided. For each analyte, plasma concentrations and PK parameters will be summarized by treatment. Diagnostic PK parameters will be summarized and listed. Tabulations will be based on the PK analysis set. Data from subjects excluded from the PK analysis set will be included in the data listings, but not in the descriptive statistics or in the inferential statistics. For each analyte, individual plasma concentration versus actual time will be plotted in linear and semi-logarithmic scales with all treatments overlaid on the same plot and separate plots for each subject. Combined individual plasma concentration versus actual times will be plotted in linear and semi-logarithmic scales with separate plots for each treatment and analyte. Geometric mean plasma concentration versus nominal sampling time will be plotted in linear scale (-/+geometric SD) and semi-logarithmic scale (no geometric SD presented) with all treatments overlaid on the same figure and separate figures for each analyte. All plots will be based on the PK analysis set, with the exception of individual plots by subject which will be based on the safety analysis set.

### Statistical Analysis of Pharmacokinetic Data:

Bioequivalence will be assessed between Treatment A: AMT (Test) versus Treatment B: Acalabrutinib capsule (Reference) based on the PK analysis set. Analyses will be performed using a linear mixed effects analysis of variance model using the natural logarithm of Cₘₐₓ, AUCₗₐₛₜ, and AUC_{inf} for acalabrutinib as the response variables, with sequence, period, treatment as fixed effects and subject nested within sequence as random effect. Transformed back from the logarithmic scale, geometric means together with confidence intervals (CIs) (2-sided 95%) for Cₘₐₓ, AUCₗₐₛₜ, and AUC_{inf} will be estimated and presented. Also, ratios of geometric means together with CIs (2-sided 90%) will be estimated and presented. In addition, the inter- and intra-%CV will be estimated and presented for Cₘₐₓ, AUC_{inf}, and AUCₗₐₛₜ for acalabrutinib and ACP-5862, respectively.

### Bioequivalence Criteria:

If the 90% CI for the log transformed geometric mean ratio of Cₘₐₓ and AUCₗₐₛₜ or AUC_{inf} between the test and reference are entirely contained within 80.00% and 125.00%, it will be concluded that the two treatments are bioequivalent. Statistical analysis for establishing bioequivalence will be conducted by combining PK data across all study centers.

### Presentation and Analysis of Pharmacodynamic Data:

The exploratory PD parameter (BTK receptor occupancy) results will be listed and summarized as appropriate, based on the pharmacokinetic analysis set.

### Determination of Sample Size:

Based on a bioequivalence range of 80.00% to 125.00% for Cₘₐₓ and AUC_{inf} for acalabrutinib, a within-subject CV of 29.8% for Cmax and 15.1% for AUC_{inf} (Study ACE-HV-115) and a “test/reference” mean ratio of 0.95, 52 evaluable subjects are needed to achieve a power of 90%.

Overall, a total of 64 subjects will provide at least 95% power to conclude bioequivalence with respect to each of Cₘₐₓ and AUC_{inf}, respectively.

### VIII. Embodiments

Embodiment 1: A solid pharmaceutical dosage form comprising from about 75 mg to about 125 mg (free base equivalent weight) of acalabrutinib maleate and at least one pharmaceutically acceptable excipient for oral administration to a human, wherein the dosage form satisfies the following conditions: (i) at least about 75% of the acalabrutinib maleate is dissolved within about 30 minutes as determined in an in vitro dissolution test conducted using a USP Dissolution Apparatus 2 (Paddle Apparatus), 900 mL dissolution volume, 0.1 N hydrochloric acid dissolution medium, and paddle rotation of 50 RPM; and (ii) at least about 75% of the acalabrutinib maleate is dissolved within about 60 minutes as determined in an in vitro dissolution test conducted using a USP Dissolution Apparatus 2 (Paddle Apparatus), 900 mL dissolution volume, 5 mM phosphate pH 6.8 dissolution medium, and paddle rotation of 75 RPM.

Embodiment 2: The dosage form of Embodiment 1, wherein the dosage form satisfies the following conditions: (i) at least about 75% of the acalabrutinib maleate is dissolved within about 20 minutes as determined in an in vitro dissolution test conducted using a USP Dissolution Apparatus 2 (Paddle Apparatus), 900 mL dissolution volume, 0.1 N hydrochloric acid dissolution medium, and paddle rotation of 50 RPM; and (ii) at least about 75% of the acalabrutinib maleate is dissolved within about 45 minutes as determined in an in vitro dissolution test conducted using a USP Dissolution Apparatus 2 (Paddle Apparatus), 900 mL dissolution volume, 5 mM phosphate pH 6.8 dissolution medium, and paddle rotation of 75 RPM.

Embodiment 3: The dosage form of Embodiment 1, wherein the dosage form satisfies the following conditions: (i) at least about 80% of the acalabrutinib maleate is dissolved within about 20 minutes as determined in an in vitro dissolution test conducted using a USP Dissolution Apparatus 2 (Paddle Apparatus), 900 mL dissolution volume, 0.1 N hydrochloric acid dissolution medium, and paddle rotation of 50 RPM; and (ii) at least about 80% of the acalabrutinib maleate is dissolved within about 30 minutes as determined in an in vitro dissolution test conducted using a USP Dissolution Apparatus 2 (Paddle Apparatus), 900 mL dissolution volume, 5 mM phosphate pH 6.8 dissolution medium, and paddle rotation of 75 RPM.

Embodiment 4: The dosage form of Embodiment 1, wherein the dosage form satisfies the following conditions: (i) at least about 80% of the acalabrutinib maleate is dissolved within about 15 minutes as determined in an in vitro dissolution test conducted using a USP Dissolution Apparatus 2 (Paddle Apparatus), 900 mL dissolution volume, 0.1 N hydrochloric acid dissolution medium, and paddle rotation of 50 RPM; and (ii) at least about 80% of the acalabrutinib maleate is dissolved within about 20 minutes as determined in an in vitro dissolution test conducted using a USP Dissolution Apparatus 2 (Paddle Apparatus), 900 mL dissolution volume, 5 mM phosphate pH 6.8 dissolution medium, and paddle rotation of 75 RPM.

Embodiment 5: The dosage form of any of Embodiments 1 to 4, wherein the acalabrutinib maleate is acalabrutinib maleate monohydrate.

Embodiment 6: The dosage form of Embodiment 5, wherein the acalabrutinib maleate monohydrate is crystalline Form A.

Embodiment 7: The dosage form of any of Embodiments 1 to 6, wherein the at least one pharmaceutically acceptable excipient is selected from at least one diluent, at least one disintegrant, and at least one lubricant.

Embodiment 8: The dosage form of any of Embodiments 1 to 7, wherein the dissolution rate of the acalabrutinib maleate in the 5 mM phosphate pH 6.8 dissolution medium does not decrease by more than 20% from its initial dissolution rate after storage of the dosage form in appropriate packaging for six months at 40° C. and 75% relative humidity.

Embodiment 9: The dosage form of any of Embodiments 1 to 7, wherein the dissolution rate of the acalabrutinib maleate in the 5 mM phosphate pH 6.8 dissolution medium does not decrease by more than 10% from its initial dissolution rate after storage of the dosage form in appropriate packaging for six months at 40° C. and 75% relative humidity.

Embodiment 10: The dosage form of any of Embodiments 1 to 7, wherein the dissolution rate of the acalabrutinib maleate in the 5 mM phosphate pH 6.8 dissolution medium does not decrease by more than 5% from its initial dissolution rate after storage of the dosage form in appropriate packaging for six months at 40° C. and 75% relative humidity.

Embodiment 11: The dosage form of any of Embodiments 1 to 7, wherein the dissolution rate of the acalabrutinib maleate in the 5 mM phosphate pH 6.8 dissolution medium does not decrease by more than 2% from its initial dissolution rate after storage of the dosage form in appropriate packaging for six months at 40° C. and 75% relative humidity.

Embodiment 12: The dosage form of any of Embodiments 1 to 11, wherein no more than about 5% (w/w) of the acalabrutinib maleate present in the dosage form degrades after storage of the dosage form in appropriate packaging for six months at 40° C. and 75% relative humidity.

Embodiment 13: The dosage form of any of Embodiments 1 to 11, wherein no more than about 2% (w/w) of the acalabrutinib maleate present in the dosage form degrades after storage of the dosage form in appropriate packaging for six months at 40° C. and 75% relative humidity.

Embodiment 14: The dosage form of any of Embodiments 1 to 11, wherein no more than about 1% (w/w) of the acalabrutinib maleate present in the dosage form degrades after storage of the dosage form in appropriate packaging for six months at 40° C. and 75% relative humidity.

Embodiment 15: The dosage form of any of Embodiments 1 to 11, wherein no more than about 0.5% (w/w) of the acalabrutinib maleate present in the dosage form degrades after storage of the dosage form appropriate packaging for six months at 40° C. and 75% relative humidity.

Embodiment 16: The dosage form of any of Embodiments 1 to 15, wherein the dosage form is bioequivalent to a 100 mg Calquence® capsule when orally administered to a fasting human subject who has not been administered a gastric acid reducing agent, wherein the dosage form is bioequivalent when the confidence interval of the relative mean Cmax, AUC(0-t), and AUC(0-∞) of the dosage form relative to the 100 mg Calquence® capsule is within 80% to 125%.

Embodiment 17: The dosage form of any of Embodiments 1 to 15, wherein the dosage form, when administered twice daily to a population of fasting human subjects, satisfies one or more of the following pharmacokinetic conditions for acalabrutinib: (i) the average Cmax value in the population of human subjects is from about 400 ng/mL to about 900 ng/mL; (ii) the average AUC(0-24) value in the population of human subjects is from about 350 ng•hr/mL to about 1900 ng•hr/mL; and/or (iii) the average AUC(0 - ∞) value in the population of human subjects is from about 350 ng•hr/mL to about 1900 ng•hr/mL.

Embodiment 18: The dosage form of Embodiment 17, wherein the dosage form is co-administered to the population of human subjects with a gastric acid reducing agent.

Embodiment 19: The dosage form of any of Embodiments 1 to 18, wherein the dosage form, when administered twice daily to a human subject, provides a median steady state Bruton tyrosine kinase occupancy of at least about 90% in peripheral blood mononuclear cells.

Embodiment 20: The dosage form of any of Embodiments 1 to 18, wherein the dosage form, when administered twice daily to a human subject, provides a median steady state Bruton tyrosine kinase occupancy of at least about 95% in peripheral blood mononuclear cells.

Embodiment 21: The dosage form of Embodiment 19 or 20, wherein the dosage form is co-administered to the population of human subjects with a gastric acid reducing agent.

Embodiment 22: The dosage form of any of Embodiments 1 to 21, wherein the acalabrutinib maleate is present in an amount of about 15% to about 55% by weight (free base equivalent weight) of the dosage form.

Embodiment 23: The dosage form of any of Embodiments 1 to 21, wherein the acalabrutinib maleate is present in an amount of about 20% to about 50% by weight (free base equivalent weight) of the dosage form.

Embodiment 24: The dosage form of any of Embodiments 1 to 21, wherein the acalabrutinib maleate is present in an amount of about 25% to about 50% by weight (free base equivalent weight) of the dosage form.

Embodiment 25: The dosage form of any of Embodiments 1 to 21, wherein the acalabrutinib maleate is present in an amount of about 25% to about 40% by weight (free base equivalent weight) of the dosage form.

Embodiment 26: The dosage form of any of Embodiments 1 to 25, wherein the at least one pharmaceutically acceptable excipient comprises at least one diluent.

Embodiment 27: The dosage form of Embodiment 26, wherein the at least one diluent is present in an amount from about 10% to about 70% by weight of the dosage form.

Embodiment 28: The dosage form of Embodiment 26, wherein the at least one diluent is present in an amount from about 20% to about 70% by weight of the dosage form.

Embodiment 29: The dosage form of Embodiment 26, wherein the at least one diluent is present in an amount from about 30% to about 70% by weight of the dosage form.

Embodiment 30: The dosage form of Embodiment 26, wherein the at least one diluent is present in an amount from about 40% to about 70% by weight of the dosage form.

Embodiment 31: The dosage form of any of Embodiments 26 to 30, wherein the at least one diluent does not affect the stability of the primary amine moiety of acalabrutinib.

Embodiment 32: The dosage form of any of Embodiments 26 to 30, wherein the at least one diluent does not comprise lactose.

Embodiment 33: The dosage form of any of Embodiments 26 to 32, wherein the at least one diluent does not comprise a maleic acid scavenging agent.

Embodiment 34: The dosage form of any of Embodiments 26 to 33, wherein the at least one diluent does not comprise dibasic calcium phosphate anhydrous.

Embodiment 35: The dosage form of any of Embodiments 26 to 34, wherein the at least one diluent comprises a plastic diluent and a brittle diluent.

Embodiment 36: The dosage form of Embodiment 35, wherein the w/w ratio of plastic diluent to brittle diluent is from about 0: 100 to about 60:40.

Embodiment 37: The dosage form of Embodiment 35 or 36, wherein: (i) the at least one diluent comprises a plastic diluent and a brittle diluent in a total amount from about 10% to about 70% by weight of the dosage form; (ii) the plastic diluent is present in an amount from about 0% to about 70% by weight of the dosage form; and (iii) the brittle diluent is present in an amount from about 0% to about 50% by weight of the dosage form.

Embodiment 38: The dosage form of any of Embodiments 26 to 34, wherein the at least one diluent comprises mannitol.

Embodiment 39: The dosage form of any of Embodiments 26 to 34, wherein the at least one diluent comprises microcrystalline cellulose.

Embodiment 40: The dosage form of any of Embodiments 26 to 34, wherein the at least one diluent comprises mannitol and microcrystalline cellulose.

Embodiment 41: The dosage form of Embodiment 40, wherein the w/w ratio of mannitol to microcrystalline cellulose is from about 0:100 to about 60:40.

Embodiment 42: The dosage form of Embodiment 38, wherein the mannitol is present in an amount from about 10% to about 70% by weight of the dosage form.

Embodiment 43: The dosage form of Embodiment 39, wherein the microcrystalline cellulose is present in an amount from about 5% to about 50% by weight of the dosage form.

Embodiment 44: The dosage form of Embodiment 40, wherein: (i) the mannitol is present in an amount from about 0% to about 70% by weight of the dosage form; (ii) the microcrystalline cellulose is present in an amount from about 0% to about 50% by weight of the dosage form; and (iii) the total amount of mannitol and microcrystalline cellulose is from about 10% to about 70% by weight of the dosage form.

Embodiment 45: The dosage form of any of Embodiments 26 to 44, wherein the weight ratio of acalabrutinib maleate (free base equivalent weight) to the at least one diluent is from about 1:3 to about 2:1.

Embodiment 46: The dosage form of any of Embodiments 26 to 44, wherein the weight ratio of acalabrutinib maleate (free base equivalent weight) to the at least one diluent is from about 1:1 to about 1:2.

Embodiment 47: The dosage form of any of Embodiments 1 to 46, wherein the at least one pharmaceutically acceptable excipient comprises at least one disintegrant.

Embodiment 48: The dosage form of Embodiment 47, wherein the at least one disintegrant is present in an amount from about 0.5% to about 15% by weight of the tablet.

Embodiment 49: The dosage form of Embodiment 47, wherein the at least one disintegrant is present in an amount from about 1% to about 10% by weight of the tablet.

Embodiment 50: The dosage form of Embodiment 47, wherein the at least one disintegrant is present in an amount from about 2% to about 8% by weight of the tablet.

Embodiment 51: The dosage form of Embodiment 47, wherein the at least one disintegrant is present in an amount from about 3% to about 7% by weight of the tablet.

Embodiment 52: The dosage form of any of Embodiments 47 to 51, wherein the at least one disintegrant does not comprise an ionic disintegrant.

Embodiment 53: The dosage form of any of Embodiments 47 to 51, wherein the at least one disintegrant does not comprise sodium starch glycolate.

Embodiment 54: The dosage form of any of Embodiments 47 to 53, wherein the at least one disintegrant does not comprise croscarmellose sodium.

Embodiment 56: The dosage form of any of Embodiments 47 to 54, wherein the at least one disintegrant comprises a non-ionic disintegrant.

Embodiment 57: The dosage form of any of Embodiments 47 to 56, wherein the at least one disintegrant comprises hydroxypropyl cellulose.

Embodiment 58: The dosage form of any of Embodiments 47 to 56, wherein the at least one disintegrant comprises low-substituted hydroxypropyl cellulose.

Embodiment 59: The dosage form of any of Embodiments 47 to 59, wherein the weight ratio of acalabrutinib maleate (free base equivalent weight) to the at least one disintegrant is from about 2:1 to about 15:1.

Embodiment 60: The dosage form of any of Embodiments 47 to 59, wherein the weight ratio of acalabrutinib maleate (free base equivalent weight) to the at least one disintegrant is from about 4:1 to about 10:1.

Embodiment 61: The dosage form of any of Embodiments 1 to 60, wherein the at least one pharmaceutically acceptable excipient comprises at least one lubricant.

Embodiment 62: The dosage form of Embodiment 61, wherein the at least one lubricant is present in an amount from about 0.25% to about 4% by weight of the dosage form.

Embodiment 63: The dosage form of Embodiment 61, wherein the at least one lubricant is present in an amount from about 1% to about 4% by weight of the dosage form.

Embodiment 64: The dosage form of any of Embodiment 61, wherein the at least one lubricant is present in an amount from about 1.5% to about 3.5% by weight of the dosage form.

Embodiment 65: The dosage form of any of Embodiment 61, wherein the at least one lubricant is present in an amount from about 2% to about 3% by weight of the dosage form.

Embodiment 66: The dosage form of any of Embodiments 61 to 65, wherein the at least one lubricant does not comprise magnesium stearate.

Embodiment 67: The dosage form of any of Embodiments 51 to 66, wherein the at least one lubricant does not comprise glyceryl dibehenate.

Embodiment 68: The dosage form of any of Embodiments 61 to 67, wherein the at least one lubricant comprises sodium stearyl fumarate.

Embodiment 69: The dosage form of any of Embodiments 61 to 68, wherein the weight ratio of acalabrutinib maleate (free base equivalent weight) to the at least one lubricant is from about 20:1 to about 12:1.

Embodiment 70: The dosage form of any of Embodiments 61 to 68, wherein the weight ratio of acalabrutinib maleate (free base equivalent weight) to the at least one lubricant is from about 18:1 to about 14:1.

Embodiment 71: The dosage form of any of Embodiments 1 to 70, wherein the at least one pharmaceutically acceptable excipient comprises at least one diluent, at least one disintegrant, and at least one lubricant.

Embodiment 72: The dosage form of Embodiment 7, wherein the dosage form comprises: (i) acalabrutinib maleate in an amount from about 15% to about 55% by weight (free base equivalent weight) of the dosage form; (ii) at least one diluent in an amount from about 10% to about 70% by weight of the dosage form; (iii) at least one disintegrant in an amount from about 0.5% to about 15% by weight of the dosage form; and (iv) at least one lubricant in an amount from about 0.25% to about 4% by weight of the dosage form; and wherein the sum of the individual amounts equals 100% of the total weight of the dosage form.

Embodiment 73: The dosage form of Embodiment 7, wherein the dosage form comprises: (i) acalabrutinib maleate in an amount from about 20% to about 50% by weight (free base equivalent weight) of the dosage form; (ii) at least one diluent in an amount from about 20% to about 70% by weight of the dosage form; (iii) at least one disintegrant in an amount from about 1% to about 10% by weight of the dosage form; and (iv) at least one lubricant in an amount from about 1% to about 4% by weight of the dosage form; and wherein the sum of the individual amounts equals 100% of the total weight of the dosage form.

Embodiment 74: The dosage form of Embodiment 7, wherein the dosage form comprises: (i) acalabrutinib maleate in an amount from about 25% to about 50% by weight (free base equivalent weight) of the dosage form; (ii) at least one diluent in an amount from about 30% to about 70% by weight of the dosage form; (iii) at least one disintegrant in an amount from about 2% to about 8% by weight of the dosage form; and (iv) at least one lubricant in an amount from about 1.5% to about 3.5% by weight of the dosage form; and wherein the sum of the individual amounts equals 100% of the total weight of the dosage form.

Embodiment 75: The dosage form of Embodiment 7, wherein the dosage form comprises: (i) acalabrutinib maleate in an amount from about 25% to about 40% (free base equivalent weight) by weight of the dosage form; (ii) at least one diluent in an amount from about 40% to about 70% by weight of the dosage form; (iii) at least one disintegrant in an amount from about 3% to about 7% by weight of the dosage form; and (iv) at least one lubricant in an amount from about 2% to about 3% by weight of the dosage form; and wherein the sum of the individual amounts equals 100% of the total weight of the dosage form.

Embodiment 76: The dosage form of Embodiment 7, wherein the dosage form comprises: (i) acalabrutinib maleate in an amount from about 30% to about 35% by weight (free base equivalent weight) of the dosage form; (ii) mannitol in an amount from about 30% to about 35% by weight of the dosage form; (iii) microcrystalline cellulose in an amount from about 25% to about 30% by weight of the dosage form; (iv) hydroxypropyl cellulose in an amount from about 3% to about 7% by weight of the dosage form; and (v) sodium stearyl fumarate in an amount from about 1% to about 4% by weight of the dosage form; and wherein the sum of the individual amounts equals 100% of the total weight of the dosage form.

Embodiment 77: The dosage form of any of Embodiments 1 to 76, wherein the acalabrutinib maleate has a D(v, 0.9) value below about 500 microns.

Embodiment 78: The dosage form of any of Embodiments 1 to 76, wherein the acalabrutinib maleate has a D(v, 0.9) value below about 450 microns.

Embodiment 79: The dosage form of any of Embodiments 1 to 76, wherein the acalabrutinib maleate has a D(v, 0.9) value below about 400 microns.

Embodiment 80: The dosage form of any of Embodiments 1 to 76, wherein the acalabrutinib maleate has a D(v, 0.9) value below about 350 microns.

Embodiment 81: The dosage form of any of Embodiments 1 to 76, wherein the acalabrutinib maleate has a D(v, 0.9) value below about 300 microns.

Embodiment 82: The dosage form of any of Embodiments 1 to 76, wherein the acalabrutinib maleate has a D(v, 0.9) value from about 20 microns to about 500 microns.

Embodiment 83: The dosage form of any of Embodiments 1 to 76, wherein the acalabrutinib maleate has a D(v, 0.9) value from about 50 microns to about 450 microns.

Embodiment 84: The dosage form of any of Embodiments 1 to 76, wherein the acalabrutinib maleate has a D(v, 0.9) value from about 75 microns to about 400 microns.

Embodiment 85: The dosage form of any of Embodiments 1 to 76, wherein the acalabrutinib maleate has a D(v, 0.9) value from about 75 microns to about 350 microns.

Embodiment 86: The dosage form of any of Embodiments 1 to 76, wherein the acalabrutinib maleate has a D(v, 0.9) value from about 100 microns to about 300 microns.

Embodiment 87: The dosage form of any of Embodiments 1 to 86, wherein the dosage form is a capsule.

Embodiment 88: The capsule of Embodiment 87, wherein the capsule is prepared by a process comprising roller compaction.

Embodiment 89: The dosage form of any of Embodiments 1 to 86, wherein the dosage form is a tablet.

Embodiment 90: The dosage form of any of Embodiments 1 to 86, wherein the dosage form is a film-coated tablet.

Embodiment 91: The tablet of Embodiment 89 or 90, wherein the tablet is prepared by a process comprising direct compression.

Embodiment 92: The tablet of Embodiment 89 or 90, wherein the tablet is prepared by a process comprising roller compaction.

Embodiment 93: The tablet of any of Embodiments 89 to 92, wherein the tablet has a tensile strength from about 1.5 MPa to about 5.0 MPa.

Embodiment 94: The tablet of any of Embodiments 89 to 92, wherein the tablet has a tensile strength from about 2.0 MPa to about 4.0 MPa.

Embodiment 95: The tablet of any of Embodiments 89 to 94, wherein the tablet tensile strength does not decrease by more than 10% from its initial tensile strength after storage of the tablet in a blister pack for six months at 40° C. and 75% relative humidity.

Embodiment 96: The tablet of any of Embodiments 89 to 94, wherein the tablet tensile strength does not decrease by more than 8% from its initial tensile strength after storage of the tablet in a blister pack for six months at 40° C. and 75% relative humidity.

Embodiment 97: The tablet of any of Embodiments 89 to 94, wherein the tablet tensile strength does not decrease by more than 5% from its initial tensile strength after storage of the tablet in a blister pack for six months at 40° C. and 75% relative humidity.

Embodiment 98: A method of treating a BTK-mediated condition in a subject suffering from or susceptible to the condition, comprising administering once or twice daily to the subject the solid pharmaceutical dosage form of any of Embodiments 1 to 97.

This written description uses examples to disclose the invention and to enable any person skilled in the art to practice the invention, including making and using any of the disclosed salts, substances, or compositions, and performing any of the disclosed methods or processes. The patentable scope of the invention is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims. While preferred embodiments of the invention are shown and described in this specification, such embodiments are provided by way of example only and are not intended to otherwise limit the scope of the invention. Various alternatives to the described embodiments of the invention may be employed in practicing the invention. Section headings as used in this section and the entire disclosure are not intended to be limiting.

All references (patent and non-patent) cited above are incorporated by reference into this patent application. The discussion of those references is intended merely to summarize the assertions made by their authors. No admission is made that any reference (or a portion of any reference) is relevant prior art (or prior art at all). Applicants reserve the right to challenge the accuracy and pertinence of the cited references.

What is claimed is:

1. A solid pharmaceutical dosage form comprising from about 75 mg to about 125 mg (free base equivalent weight) of acalabrutinib maleate and at least one pharmaceutically acceptable excipient for oral administration to a human, wherein the dosage form satisfies the following conditions:
at least about 75% of the acalabrutinib maleate is dissolved within about 30 minutes as determined in an in vitro dissolution test conducted using a USP Dissolution Apparatus 2 (Paddle Apparatus), 900 mL dissolution volume, 0.1 N hydrochloric acid dissolution medium, and paddle rotation of 50 RPM; and
at least about 75% of the acalabrutinib maleate is dissolved within about 60 minutes as determined in an in vitro dissolution test conducted using a USP Dissolution Apparatus 2 (Paddle Apparatus), 900 mL dissolution volume, 5 mM phosphate pH 6.8 dissolution medium, and paddle rotation of 75 RPM.

2. The dosage form of claim 1, wherein the dosage form satisfies the following conditions:
at least about 80% of the acalabrutinib maleate is dissolved within about 15 minutes as determined in an in vitro dissolution test conducted using a USP Dissolution Apparatus 2 (Paddle Apparatus), 900 mL dissolution volume, 0.1 N hydrochloric acid dissolution medium, and paddle rotation of 50 RPM; and
at least about 80% of the acalabrutinib maleate is dissolved within about 20 minutes as determined in an in vitro dissolution test conducted using a USP Dissolution Apparatus 2 (Paddle Apparatus), 900 mL dissolution volume, 5 mM phosphate pH 6.8 dissolution medium, and paddle rotation of 75 RPM.

3. The dosage form of claim 1 or 2, wherein the acalabrutinib maleate is crystalline acalabrutinib maleate monohydrate Form A.

4. The dosage form of any of claims 1 to 3, wherein the at least one pharmaceutically acceptable excipient is selected from at least one diluent, at least one disintegrant, and at least one lubricant.

5. The dosage form of any of claims 1 to 4, wherein the dissolution rate of the acalabrutinib maleate in the 5 mM phosphate pH 6.8 dissolution medium does not decrease by more than 20% from its initial dissolution rate after storage of the dosage form in appropriate packaging for six months at 40° C. and 75% relative humidity.

6. The dosage form of any of claims 1 to 5, wherein no more than about 5% (w/w) of the acalabrutinib maleate present in the dosage form degrades after storage of the dosage form in appropriate packaging for six months at 40° C. and 75% relative humidity.

7. The dosage form of any of claims 1 to 6, wherein the dosage form is bioequivalent to a 100 mg Calquence® capsule when orally administered to a fasting human subject who has not been administered a gastric acid reducing agent, wherein the dosage form is bioequivalent when the confidence interval of the relative mean Cₘₐₓ, AUC₍₀₋ₜ₎, and AUC_{(0-∞)} of the dosage form relative to the 100 mg Calquence® capsule is within 80% to 125%.

8. The dosage form of any of claims 1 to 7, wherein the acalabrutinib maleate is present in an amount of about 100 mg (free base equivalent weight).

9. The dosage form of any of claims 1 to 8, wherein the at least one pharmaceutically acceptable excipient comprises at least one diluent.

10. The dosage form of claim 9, wherein the at least one diluent does not affect the stability of the primary amine moiety of acalabrutinib.

11. The dosage form of claim 9 or 10, wherein the at least one diluent comprises a plastic diluent and a brittle diluent.

12. The dosage form of any of claims 9 to 11, wherein the weight ratio of acalabrutinib maleate (free base equivalent weight) to the at least one diluent is from about 1:3 to about 2:1.

13. The dosage form of any of claims 1 to 12, wherein the at least one pharmaceutically acceptable excipient comprises at least one disintegrant.

14. The dosage form of claim 13, wherein the at least one disintegrant does not comprise an ionic disintegrant.

15. The dosage form of claim 13 or 14, wherein the weight ratio of acalabrutinib maleate (free base equivalent weight) to the at least one disintegrant is from about 2:1 to about 15:1.

16. The dosage form of claim 4, wherein the dosage form comprises:
acalabrutinib maleate in an amount from about 15% to about 55% by weight (free base equivalent weight) of the dosage form;
at least one diluent in an amount from about 10% to about 70% by weight of the dosage form;
at least one disintegrant in an amount from about 0.5% to about 15% by weight of the dosage form; and
at least one lubricant in an amount from about 0.25% to about 4% by weight of the dosage form; and
wherein the sum of the individual amounts equals 100% of the total weight of the dosage form.

17. The dosage form of claim 4, wherein the dosage form comprises:
acalabrutinib maleate in an amount from about 30% to about 35% by weight (free base equivalent weight) of the dosage form; and
mannitol in an amount from about 30% to about 35% by weight of the dosage form;
microcrystalline cellulose in an amount from about 25% to about 30% by weight of the dosage form;
hydroxypropyl cellulose in an amount from about 3% to about 7% by weight of the dosage form; and
sodium stearyl fumarate in an amount from about 1% to about 4% by weight of the dosage form; and
wherein the sum of the individual amounts equals 100% of the total weight of the dosage form.

18. The dosage form of any of claims 1 to 17, wherein the acalabrutinib maleate has a D_{(v, 0.9)} value from about 20 microns to about 500 microns.

19. The dosage form of any of claims 1 to 18, wherein the dosage form is a tablet.

20. The tablet of claim 19, wherein the tablet has a tensile strength from about 1.5 MPa to about 5.0 MPa.

21. The tablet of claim 20, wherein the tablet tensile strength does not decrease by more than 10% from its initial tensile strength after storage of the tablet in a blister pack for six months at 40° C. and 75% relative humidity.

22. A method of treating a BTK-mediated condition in a subject suffering from or susceptible to the condition, comprising administering once or twice daily to the subject the solid pharmaceutical dosage form of any of claims 1 to 21.
